(19) United States
(12) Patent Application Publication
(10) Pub. No.: US 2023/0407273 A1
(43) Pub. Date: Dec. 21, 2023

(54) GLYCOSYLTRANSFERASE VARIANTS FOR IMPROVED PROTEIN PRODUCTION

(71) Applicant: Novozymes A/S, Bagsvaerd (DK)

(72) Inventors: Michiko Ihara, Makuhari-hongo (JP); Takashi Nakanishi, Chiba-shi (JP)

(73) Assignee: Novozymes A/S, Bagsvaerd (DK)

(21) Appl. No.: 18/249,037

(22) PCT Filed: Oct. 8, 2021
(86) PCT No.: PCT/EP2021/077919

§ 371 Date: Apr. 13, 2023

(30) Foreign Application Priority Data
Oct. 13, 2020 (DK) PA 2020 01167

(51) Int. Cl. C12N 9/10; C12N 15/80; C12N 9/34; C12N 9/36

(52) U.S. Cl. CPC C12N 9/1048; C12N 15/80; C12N 9/2428; C12N 9/2462; C12R 2001/66

(57) ABSTRACT

The present invention relates to a polynucleotide variant encoding a glycosyltransferase variant, and to nucleic acid constructs, vectors and host cells comprising said polynucleotide variant. The invention also relates to methods of producing a polypeptide of interest in host cells comprising said polynucleotide and/or glycosyltransferase variant.

### REFERENCE TO A SEQUENCE LISTING

This application contains a Sequence Listing in computer readable form, which is incorporated herein by reference.

### BACKGROUND OF THE INVENTION

### Field of the Invention

The present invention relates to polynucleotide variants encoding glycosyltransferase variants, and to nucleic acid constructs, vectors and host cells comprising said polynucleotide variants, and to host cells comprising said glycosyltransferase variant, as well as methods of producing a polypeptide of interest in host cells comprising said polynucleotide and/or glycosyltransferase variant.

### Description of the Related Art

Recombinant gene expression in fungal hosts is a common method for recombinant protein production. Recombinant proteins produced in such fungal systems are enzymes and other valuable proteins. In industrial and commercial purposes, the productivity of the applied cell systems, i.e. the production of total protein per fermentation unit, is an important factor of production costs. Traditionally, yield increases have been achieved through mutagenesis and screening for increased production of proteins of interest. However, this approach is mainly only useful for the overproduction of endogenous proteins in isolates containing the enzymes of interest. Therefore, for each new protein or enzyme product, a lengthy strain and process development program is required to achieve improved productivities.

For the overexpression of heterologous proteins in fungal systems, the production process is recognized as a complex multi-phase and multi-component process. Cell growth and product formation are determined by a wide range of parameters, including the composition of the culture medium, fermentation pH, fermentation temperature, dissolved oxygen tension, shear stress, and fungal morphology.

Various approaches to improve transcription have been used in fungi. For the expression of heterologous genes, codon-optimized, synthetic genes can improve the transcription rate. To obtain high-level expression of a particular gene, a well-established procedure is targeting multiple copies of the recombinant gene constructs to the locus of a highly expressed endogenous gene. Also, for the secretion of foreign proteins, fusion strategies are used to facilitate translocation in the secretion pathway and to protect the heterologous protein from degrading. A further strategy for reducing the proteolytic degradation of recombinant proteins by the disruption of native proteases and thereby improving protein yield is described in WO 2011/075677 (Novozymes A/S). Despite the presented approaches, it is of continuous interest to further improve recombinant protein production in fungal host cells.

The object of the present invention is to provide a modified fungal host strain and a method of protein production with increased productivity of recombinant protein.

### SUMMARY OF THE INVENTION

The present invention is based on the surprising and inventive finding that certain single nucleotide polymorphisms (SNPs) in the alg3 gene in fungal host cells provide an improved expression, activity and/or yield of heterologous proteins compared to expression of the same heterologous proteins in fungal host cells with native alg3 gene. Surprisingly, the inventors have found Alg3 amino acid substitutions to create host cell variants which show improved product yields, without inactivating Alg3 glycosylation activities.

The alg3 gene encodes a Man₅GlcNAc₂-PP-Dol alpha-1,3-mannosyltransferase denoted Alg3 that is involved in N-glycosylation of proteins. Alg3 catalyzes the addition of the first dol-P-Man derived mannose in an alpha 1,3-linkage to Man₅GlcNAc₂-PP-Dol, resulting in MansGlcNAc₂-PP-Dol. In humans, defects in the alg3 gene have been associated with congenital disorder of glycosylation type Id (CDG-Id) characterized by abnormal N-glycosylation. Surprisingly, by mutation of the alg3 gene in fungal host cells, the present invention results in increased productivity and/or activity of recombinant protein, such as glucoamylase and lysozyme productivity and/or activity. As can be seen from the Examples below, an Aspergillus niger host cell carrying a variant of the alg3 gene that expresses an R15*, T17I and/or L137F mutant of Alg3 provides improved activity and/or yield of a variant of the Gloeophyllum sepiarium glucoamylase (SEQ ID NO: 9, see also Example 1 in WO2018/191215). As can be further seen from the Examples below, a Trichoderma reesei host cell carrying a variant of the alg3 gene that expresses an S19I and/or L139F mutant of Alg3 provides improved activity and/or yield of a variant of the Acremonium alcalophilum lysozyme. Thus, we expect that this finding also applies to other proteins, such as other glycoproteins, and in particular to other glucoamylases and lysozymes.

Thus, in a first aspect, the present invention relates to a fungal host cell comprising in its genome a first polynucleotide encoding a polypeptide of interest; and a second polynucleotide encoding a polypeptide having a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100% to SEQ ID NO: 7 and comprising at least one alteration at a position corresponding to position 15, 17 and/or 137 of SEQ ID NO: 7.

The present invention also relates to a fungal host cell comprising in its genome a first polynucleotide encoding a polypeptide of interest; and a second polynucleotide encoding a polypeptide having a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100% to SEQ ID NO: 21 and comprising at least one alteration at a position corresponding to position 19 and/or 139 of SEQ ID NO: 21.

In a second aspect, the present invention relates to a method for producing a polypeptide of interest, the method comprising:
i) providing a fungal host cell according to the first aspect,
ii) cultivating said host cell under conditions conducive for expression of the polypeptide of interest; and, optionally
iii) recovering the polypeptide of interest.

In a third aspect, the present invention relates to a nucleic acid construct comprising a heterologous promoter operably linked to a polynucleotide encoding a polypeptide having a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100% to SEQ ID NO: 7 or SEQ ID NO: 21 and comprising at least one alteration at a position corresponding to position 15, 17 and/or 137 of SEQ ID NO: 7, or corresponding to position 19 and/or 139 of SEQ ID NO: 21.

In a final and fourth aspect, the present invention relates to an expression vector comprising a nucleic acid construct according to the third aspect of the invention.

### BRIEF DESCRIPTION OF THE FIGURES

FIG. 1 shows a schematic drawing of the plasmid plhar531.

FIG. 2 shows the MS analysis of GSA202 proteins for investigation of intact molecular weight as MS peaks in (m/z).

FIG. 3 shows a polypeptide sequence alignment between Alg3 wildtypes of A. niger and T. reesei.

FIG. 4 shows a schematic drawing of the plasmid pGMEr280.

### DEFINITIONS

In accordance with this detailed description, the following definitions apply. Note that the singular forms “a,” “an,” and “the” include plural references unless the context clearly dictates otherwise.

Reference to “about” a value or parameter herein includes aspects that are directed to that value or parameter per se. For example, description referring to “about X” includes the aspect “X”.

Unless defined otherwise or clearly indicated by context, all technical and scientific terms used herein have the same meaning as commonly understood by one of ordinary skill in the art to which this invention belongs.

Alg3: The term “Alg3” means a protein with Man₅GlcNAc₂-PP-Dol alpha-1,3-mannosyltransferase activity (EC number 2.4.1.258) that catalyzes the addition of the first dol-P-Man derived mannose in an alpha 1,3-linkage to Man₅GlcNAc₂-PP-Dol, resulting in MansGlcNAc₂-PP-Dol. The Alg3 protein is encoded by the alg3 gene. According to the invention, the Alg3 protein with an alteration at a position corresponding to position 15, 17 and/or 137 of SEQ ID NO: 7, or corresponding to position 19 and/or 139 of SEQ ID NO: 21, shows an altered, reduced or eliminated Man₅GlcNAc₂-PP-Dol alpha-1,3-mannosyltransferase activity compared to the native Alg3 protein.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon, such as ATG, GTG, or TTG, and ends with a stop codon, such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a polypeptide of the present invention. Each control sequence may be native (i.e., from the same gene) or heterologous (i.e., from a different gene) to the polynucleotide encoding the polypeptide or native or heterologous to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” means any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Fusion polypeptide: The term “fusion polypeptide” is a polypeptide in which one polypeptide is fused at the N-terminus or the C-terminus of a polypeptide of the present invention. A fusion polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779). A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

Glucoamylase: The term “glucoamylase” means a protein with glucoamylase activity (EC number 3.2.1.3) that catalyzes the hydrolysis of terminal (1->4)-linked alpha-D-glucose residues successively from non-reducing ends of the chains with release of beta-D-glucose. For purposes of the present invention, glucoamylase activity is determined according to the procedure described in the Examples. In one aspect, the polypeptides of the present invention have at least 20%, e.g., at least 40%, at least 50%, at least 60%, at least 70%, at least 80%, at least 90%, at least 95%, or at least 100% of the glucoamylase activity of the mature polypeptide of SEQ ID NO: 9 or SEQ ID NO: 11. The term “glucoamylase” is interchangeable with the terms “amyloglucosidase”, “glucan 1,4-α-glucosidase”, and/or “γ-amylase”.

Glycoprotein: The term “glycoprotein” means a conjugated protein in which the non-protein group is a carbohydrate. Glycoproteins contain oligosaccharide chains/glycans covalently attached to polypeptide sidechains. The carbohydrate is attached to the protein during co-translational modification and/or post-translational modification. Glycoproteins can contain N-linked and/or O-linked oligosaccharide residues. Non-limiting examples for a glycoprotein are an alpha-glucosidase, such as the glucoamylase of SEQ ID NO: 9 or SEQ ID NO: 11.

Heterologous: The term “heterologous” means, with respect to a host cell, that a polypeptide or nucleic acid does not naturally occur in the host cell. The term “heterologous” means, with respect to a polypeptide or nucleic acid, that a control sequence, e.g., promoter, or domain of a polypeptide or nucleic acid is not naturally associated with the polypeptide or nucleic acid, i.e., the control sequence is from a gene other than the gene encoding the mature polypeptide of SEQ ID NO: 7 or SEQ ID NO: 21.

Host cell: The term “host cell” means any microbial or plant cell into which a nucleic acid construct or expression vector comprising a polynucleotide of the present invention has been introduced. Methods for introduction include but are not limited to protoplast fusion, transfection, transformation, electroporation, conjugation, and transduction. In some embodiments, the host cell is an isolated recombinant host cell that is partially or completely separated from at least one other component with, including but not limited to, proteins, nucleic acids, cells, etc.

Hybridization: The term “hybridization” means the pairing of substantially complementary strands of nucleic acids, using standard Southern blotting procedures. Hybridization may be performed under medium, medium-high, high or very high stringency conditions. Medium stringency conditions means prehybridization and hybridization at 42° C. in 5×SSPE, 0.3% SDS, 200 micrograms/ml sheared and denatured salmon sperm DNA, and 35% formamide for 12 to 24 hours, followed by washing three times each for 15 minutes using 0.2×SSC, 0.2% SDS at 55° C. Medium-high stringency conditions means prehybridization and hybridization at 42° C. in 5×SSPE, 0.3% SDS, 200 micrograms/ml sheared and denatured salmon sperm DNA, and 35% formamide for 12 to 24 hours, followed by washing three times each for 15 minutes using 0.2×SSC, 0.2% SDS at 60° C. High stringency conditions means prehybridization and hybridization at 42° C. in 5×SSPE, 0.3% SDS, 200 micrograms/ml sheared and denatured salmon sperm DNA, and 50% formamide for 12 to 24 hours, followed by washing three times each for 15 minutes using 0.2×SSC, 0.2% SDS at 65° C. Very high stringency conditions means prehybridization and hybridization at 42° C. in 5×SSPE, 0.3% SDS, 200 micrograms/ml sheared and denatured salmon sperm DNA, and 50% formamide for 12 to 24 hours, followed by washing three times each for 15 minutes using 0.2×SSC, 0.2% SDS at 70° C.

Isolated: The term “isolated” means a polypeptide, nucleic acid, cell, or other specified material or component that is separated from at least one other material or component with which it is naturally associated as found in nature, including but not limited to, for example, other proteins, nucleic acids, cells, etc. An isolated polypeptide includes, but is not limited to, a culture broth containing the secreted polypeptide.

Lysozyme: The term “lysozyme” means a protein with lysosyme activity (EC number 3.2.1.17) that catalyzes the hydrolysis of O- and S-glycosyl compounds. For purposes of the present invention, lysozyme activity is determined according to the procedure described in the examples. In one aspect, the polypeptides of the present invention have at least 20%, e.g., at least 40%, at least 50%, at least 60%, at least 70%, at least 80%, at least 90%, at least 95%, or at least 100% of the lysozyme activity of the mature polypeptide of SEQ ID NO: 33. The term “lysozyme” is interchangeable with the terms “autolysin”, “globulin G”, “muramidase”, “1,4-beta-N-acetylmuramidase”, and/or “N-acetylmuramide glycanhydrolase”.

Mature polypeptide: The term “mature polypeptide” means a polypeptide in its mature form following N-terminal processing (e.g., removal of signal peptide). In one embodiment, the mature Alg3 polypeptide is SEQ ID NO: 7. In a preferred embodiment the mature Alg3 polypeptide is SEQ ID NO: 8.

Mature polypeptide coding sequence: The term “mature polypeptide coding sequence” means a polynucleotide that encodes a mature polypeptide having biological activity.

Native: The term “native” means a nucleic acid or polypeptide naturally occurring in a host cell.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Purified: The term “purified” means a nucleic acid or polypeptide that is substantially free from other components as determined by analytical techniques well known in the art (e.g., a purified polypeptide or nucleic acid may form a discrete band in an electrophoretic gel, chromatographic eluate, and/or a media subjected to density gradient centrifugation). A purified nucleic acid or polypeptide is at least about 50% pure, usually at least about 60%, about 65%, about 70%, about 75%, about 80%, about 85%, about 90%, about 91%, about 92%, about 93%, about 94%, about 95%, about 96%, about 97%, about 98%, about 99%, about 99.5%, about 99.6%, about 99.7%, about 99.8% or more pure (e.g., percent by weight on a molar basis). In a related sense, a composition is enriched for a molecule when there is a substantial increase in the concentration of the molecule after application of a purification or enrichment technique. The term “enriched” refers to a compound, polypeptide, cell, nucleic acid, amino acid, or other specified material or component that is present in a composition at a relative or absolute concentration that is higher than a starting composition.

Recombinant: The term “recombinant,” when used in reference to a cell, nucleic acid, protein or vector, means that it has been modified from its native state. Thus, for example, recombinant cells express genes that are not found within the native (non-recombinant) form of the cell, or express native genes at different levels or under different conditions than found in nature. Recombinant nucleic acids differ from a native sequence by one or more nucleotides and/or are operably linked to heterologous sequences, e.g., a heterologous promoter in an expression vector. Recombinant proteins may differ from a native sequence by one or more amino acids and/or are fused with heterologous sequences. A vector comprising a nucleic acid encoding a polypeptide is a recombinant vector. The term “recombinant” is synonymous with “genetically modified” and “transgenic”.

Sequence identity: The relatedness between two amino acid sequences or between two nucleotide sequences is described by the parameter “sequence identity”.

For purposes of the present invention, the sequence identity between two amino acid sequences is determined as the output of “longest identity” using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 6.6.0 or later. The parameters used are a gap open penalty of 10, a gap extension penalty of 0.5, and the EBLOSUM62 (EMBOSS version of BLOSUM62) substitution matrix. In order for the Needle program to report the longest identity, the -nobrief option must be specified in the command line. The output of Needle labeled “longest identity” is calculated as follows:

(Identical Residues×100)/(Length of Alignment−Total Number of Gaps in Alignment)

For purposes of the present invention, the sequence identity between two polynucleotide sequences is determined as the output of “longest identity” using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 6.6.0 or later. The parameters used are a gap open penalty of 10, a gap extension penalty of 0.5, and the EDNAFULL (EMBOSS version of NCBI NUC4.4) substitution matrix. In order for the Needle program to report the longest identity, the nobrief option must be specified in the command line. The output of Needle labeled “longest identity” is calculated as follows:

(Identical Deoxyribonucleotides×100)/(Length of Alignment−Total Number of Gaps in Alignment)

SNP: The term “SNP” means “single nucleotide polymorphism” which is a variation at a single position in a nucleotide sequence. SNPs may lead to variations in the amino acid sequence of a polypeptide encoded by a nucleotide containing one or more SNP. SNPs can both occur in coding regions (exons) and noncoding regions (introns) of DNA.

In a preferred embodiment of the present invention, a fungal host cell comprises in its genome: a first polynucleotide encoding a polypeptide of interest; and a second polynucleotide encoding a polypeptide having a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100% to SEQ ID NO: 7 or SEQ ID NO: 21 and comprising an alteration at a position corresponding to position 137 of SEQ ID NO: 7 or corresponding to position 139 of SEQ ID NO: 21, said alteration resulting from a SNP within the second polynucleotide, preferably the SNP within the second polynucleotide is an alteration at a position corresponding to position 495, 496 and/or 497 of the second polynucleotide with SEQ ID NO: 3, or corresponding to position 656, 657 and/or 658 of the second polynucleotide with SEQ ID NO: 28, wherein the second polynucleotide is a polynucleotide variant having a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100%, to SEQ ID NO: 3 or SEQ ID NO: 28.

In another preferred embodiment of the present invention, a fungal host cell comprises in its genome: a first polynucleotide encoding a polypeptide of interest; and a second polynucleotide encoding a polypeptide having a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100% to SEQ ID NO: 7 or SEQ ID NO: 21 and comprising an alteration at a position corresponding to position 17 of SEQ ID NO: 7 or corresponding to position 19 of SEQ ID NO: 21, said alteration resulting from a SNP within the second polynucleotide, preferably the SNP within the second polynucleotide is an alteration at a position corresponding to position 49, 50 and/or 51 of the second polynucleotide with SEQ ID NO: 16, or corresponding to position 55, 56, and/or 57 of SEQ ID NO: 34, wherein the second polynucleotide is a polynucleotide variant having a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100%, to SEQ ID NO: 3 or SEQ ID NO: 28.

In another preferred embodiment of the present invention, a fungal host cell comprises in its genome: a first polynucleotide encoding a polypeptide of interest; and a second polynucleotide encoding a polypeptide having a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100% to SEQ ID NO: 7 and comprising an alteration at a position corresponding to position 15 of SEQ ID NO: 7, said alteration being a premature polypeptide termination resulting from a SNP within the second polynucleotide, preferably the SNP within the second polynucleotide is an alteration at a position corresponding to position 43, 44 and/or 45 of the second polynucleotide with SEQ ID NO: 13, wherein the second polynucleotide is a polynucleotide variant having a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100%, to SEQ ID NO: 3.

Variant: In respect to polypeptides, the term “variant” means a polypeptide having altered, reduced or eliminated Man₅GlcNAc₂-PP-Dol alpha-1,3-mannosyltransferase activity comprising a man-made mutation, i.e., a substitution, insertion, premature stop codon, premature polypeptide termination and/or deletion (e.g., truncation), at one or more (e.g., several) positions. A substitution means replacement of the amino acid occupying a position with a different amino acid; a deletion means removal of one or more amino acid(s) occupying a position; and an insertion means adding one or more amino acid(s) adjacent to and immediately following the amino acid occupying a position. A premature stop-codon or premature polypeptide termination means that the corresponding amino acid is missing and that the polypeptide ends with the amino-acid corresponding to the codon directly upstream of the premature stop-codon or polypeptide termination.

In respect to polynucleotides, the term “variant” means a polynucleotide encoding a polypeptide having altered, reduced or eliminated Man₅GlcNAc₂-PP-Dol alpha-1,3-mannosyltransferase activity comprising a man-made mutation, i.e., a substitution, insertion, and/or deletion (e.g., truncation), at one or more (e.g., several) positions. The mutation may lead to an altered, reduced or eliminated Man₅GlcNAc₂-PP-Dol alpha-1,3-mannosyltransferase activity compared to the wild type protein. A substitution means replacement of the nucleotide occupying a position with a different nucleotide; a deletion means removal of one or more nucleotide(s) occupying a position; and an insertion means adding one or more nucleotide(s) adjacent to and immediately following the nucleotide occupying a position.

Wild-type: The term “wild-type” in reference to an amino acid sequence or nucleic acid sequence means that the amino acid sequence or nucleic acid sequence is a native or naturally-occurring sequence. As used herein, the term “naturally-occurring” refers to anything (e.g., proteins, amino acids, or nucleic acid sequences) that is found in nature. Conversely, the term “non-naturally occurring” refers to anything that is not found in nature (e.g., recombinant nucleic acids and protein sequences produced in the laboratory or modification of the wild-type sequence).

### DETAILED DESCRIPTION OF THE INVENTION

### Host Cells

The present invention also relates to recombinant host cells, comprising a polynucleotide of the present invention operably linked to one or more control sequences that direct the production of a polypeptide of the present invention. A construct or vector comprising a polynucleotide is introduced into a host cell so that the construct or vector is maintained as a chromosomal integrant or as a self-replicating extra-chromosomal vector as described earlier. The choice of a host cell will to a large extent depend upon the gene encoding the polypeptide and its source.

In some embodiments, the polypeptide is heterologous to the recombinant host cell.

In some embodiments, at least one of the one or more control sequences is heterologous to the polynucleotide encoding the polypeptide.

In some embodiments, the recombinant host cell comprises at least two copies, e.g., three, four, or five, of the polynucleotide of the present invention.

The host cell may be any microbial cell useful in the recombinant production of a polypeptide of the present invention, e.g., a fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Talaromyces emersonii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

In a first aspect, the invention relates to a fungal host cell comprising in its genome:
a) a first polynucleotide encoding a polypeptide of interest; and
b) a second polynucleotide encoding a polypeptide having a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100% to SEQ ID NO: 7 or SEQ ID NO: 21 and comprising an alteration at a position corresponding to position 15, 17 and/or 137 of SEQ ID NO: 7, or corresponding to position 19 and/or 139 of SEQ ID NO: 21.

In one embodiment, the alteration is an amino acid substitution.

In a preferred embodiment, the alteration of said polypeptide is an amino acid substitution at a position corresponding to position 137 of SEQ ID NO: 7, L137F.

In another preferred embodiment, the alteration of said polypeptide is an amino acid substitution at a position corresponding to position 139 of SEQ ID NO: 21, L139F.

In a preferred embodiment of the first aspect, the alteration of said polypeptide at the position corresponding to position 15, 17 and/or 137 of SEQ ID NO: 7 is, independently chosen from one another, an amino acid substitution, an amino acid insertion, an amino acid deletion or a premature polypeptide termination.

In another preferred embodiment, the alteration of said polypeptide at the position corresponding to position 17 and/or 137 of SEQ ID NO: 7 is an amino acid substitution, preferably a substitution of leucine by phenylalanine at a position corresponding to position 137 of SEQ ID NO: 7 L137F, as presented in SEQ ID NO: 8; and/or an amino acid substitution of threonine by isoleucine at a position corresponding to position 17 of SEQ ID NO: 7 T17I, as presented in SEQ ID NO: 18.

In another preferred embodiment the alteration of said polypeptide at a position corresponding to position 15 of SEQ ID NO: 7 is a premature polypeptide termination R15*, as presented in SEQ ID NO: 15.

As shown throughout the examples the inventors have surprisingly found that said substitution L137F and/or T17I of SEQ ID NO: 7, and the premature polypeptide termination R15* leads to increased recombinant enzyme productivity and/or recombinant enzyme activity when producing the recombinant enzyme in fungal host cells carrying said engineered alg13 gene.

In another embodiment, the alteration of said polypeptide at the position corresponding to position 137 of SEQ ID NO: 7 is an amino acid substitution, such as a substitution of leucine by isoleucine, valine, histidine, lysine, methionine, threonine, tryptophan, asparagine, aspartic acid/aspartate, alanine, arginine, cysteine/cystine, glutamine, phenylalanine glutamic acid/glutamate, glycine, proline, serine, or tyrosine.

In another embodiment, the alteration of said polypeptide at the position corresponding to position 17 of SEQ ID NO: 7 is an amino acid substitution, such as a substitution of threonine by isoleucine, valine, histidine, lysine, methionine, leucine, tryptophan, asparagine, aspartic acid/aspartate, alanine, arginine, cysteine/cystine, glutamine, glutamic acid/glutamate, glycine, proline, serine, phenylalanine or tyrosine.

In another embodiment the alteration of said polypeptide at the position corresponding to position 17 and/or 137 of SEQ ID NO: 7 is an amino acid insertion, such as an amino acid insertion of at least one amino acid selected from the list of leucine, phenylalanine, isoleucine, valine, histidine, lysine, methionine, threonine, tryptophan, asparagine, aspartic acid/aspartate, alanine, arginine, cysteine/cystine, glutamine, glutamic acid/glutamate, glycine, proline, serine, or tyrosine. The amino acid insertion can be an insertion of one, two, three, four, five, or more than five amino acids, wherein the amino acids are independently chosen from one another.

In a preferred embodiment of the first aspect, the alteration of said polypeptide at the position corresponding to position 19 and/or 139 of SEQ ID NO: 21 is, independently chosen from one another, an amino acid substitution, an amino acid insertion, an amino acid deletion or a premature polypeptide termination.

In another preferred embodiment, the alteration of said polypeptide at the position corresponding to position 19 and/or 139 of SEQ ID NO: 21 is an amino acid substitution, preferably a substitution of leucine by phenylalanine at a position corresponding to position 139 of SEQ ID NO: 21 L139F, as presented in SEQ ID NO: 39; and/or an amino acid substitution of serine by isoleucine at a position corresponding to position 19 of SEQ ID NO: 21 S19I, as presented in SEQ ID NO: 36.

As shown throughout the examples the inventors have surprisingly found that said substitution L139F and/or S19I of SEQ ID NO: 21 leads to increased recombinant enzyme productivity and/or recombinant enzyme activity when producing the recombinant enzyme in fungal host cells carrying said engineered alg13 gene.

In another embodiment, the alteration of said polypeptide at the position corresponding to position 139 of SEQ ID NO: 21 is an amino acid substitution, such as a substitution of leucine by isoleucine, valine, histidine, lysine, methionine, threonine, tryptophan, asparagine, aspartic acid/aspartate, alanine, arginine, cysteine/cystine, glutamine, phenylalanine glutamic acid/glutamate, glycine, proline, serine, or tyrosine.

In another embodiment, the alteration of said polypeptide at the position corresponding to position 19 of SEQ ID NO: 21 is an amino acid substitution, such as a substitution of serine by isoleucine, valine, histidine, lysine, methionine, leucine, tryptophan, asparagine, aspartic acid/aspartate, alanine, arginine, cysteine/cystine, glutamine, glutamic acid/glutamate, glycine, proline, threonine, phenylalanine or tyrosine.

In another embodiment the alteration of said polypeptide at the position corresponding to position 19 and/or 139 of SEQ ID NO: 21 is an amino acid insertion, such as an amino acid insertion of at least one amino acid selected from the list of leucine, phenylalanine, isoleucine, valine, histidine, lysine, methionine, threonine, tryptophan, asparagine, aspartic acid/aspartate, alanine, arginine, cysteine/cystine, glutamine, glutamic acid/glutamate, glycine, proline, serine, or tyrosine. The amino acid insertion can be an insertion of one, two, three, four, five, or more than five amino acids, wherein the amino acids are independently chosen from one another.

In yet another preferred embodiment, the fungal host cell is a yeast host cell; preferably the yeast host cell is selected from the group consisting of Candida, Hansenula, Kluyveromyces, Pichia (Komagataella), Saccharomyces, Schizosaccharomyces, and Yarrowia cell; more preferably the yeast host cell is selected from the group consisting of Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, and Yarrowia lipolytica cell, most preferably Pichia pastoris (Komagataella phaffii).

In yet another preferred embodiment, the fungal host cell is a filamentous fungal host cell; preferably the filamentous fungal host cell is selected from the group consisting of Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, and Trichoderma cell; more preferably the filamentous fungal host cell is selected from the group consisting of Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, and Trichoderma viride cell; even more preferably the filamentous host cell is selected from the group consisting of Aspergillus oryzae, Fusarium venenatum, and Trichoderma reesei cell; most preferably the filamentous fungal host cell is an Aspergillus niger cell.

In one preferred embodiment, the fungal host cell is an Aspergillus oryzae cell.

In yet another preferred embodiment, the fungal host cell is a Trichoderma reesei cell.

In a further preferred embodiment the polypeptide of interest comprises an enzyme; preferably the enzyme is selected from the group consisting of hydrolase, isomerase, ligase, lyase, oxidoreductase, or transferase; more preferably an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, alpha-glucosidase, beta-glucosidase, invertase, laccase, lipase, mannosidase, mutanase, nuclease, oxidase, pectinolytic enzyme, peroxidase, phosphodiesterase, phytase, polyphenoloxidase, proteolytic enzyme, ribonuclease, transglutaminase, xylanase, and beta-xylosidase.

In a preferred embodiment, the polypeptide of interest is a glycoprotein.

In another preferred embodiment, the polypeptide of interest is an alpha-glucosidase, preferably an 1,4-alpha-glucosidase, most preferably a glucoamylase.

In one preferred embodiment the polypeptide of interest is comprises, consists essentially of, or consists of SEQ ID NO: 9.

In another embodiment the polypeptide of interest is comprises, consists essentially of, or consists of SEQ ID NO: 11.

In another embodiment the polypeptide of interest is a hydrolase, preferably a glycoside hydrolase.

In yet another embodiment the polypeptide of interest is a lysozyme, preferably a lysozyme which comprises, consists essentially of, or consists of SEQ ID NO: 33.

### Methods of Production

In a second aspect, the present invention also relates to producing one or more polypeptide of interest, said method comprising the steps of:
i) providing a fungal host cell of the first aspect,
ii) cultivating said host cell under conditions conducive for expression of the polypeptide of interest; and, optionally
iii) recovering the polypeptide of interest.

In a preferred embodiment of the second aspect, the alteration of said polypeptide at the position corresponding to position 15, 17 and/or 137 of SEQ ID NO: 7, or corresponding to position 19 and/or 139 of SEQ ID NO: 21 is, independently chosen from one another, an amino acid substitution, an amino acid insertion, a premature polypeptide termination or an amino acid deletion.

In another preferred embodiment, the alteration of said polypeptide at the position corresponding to position 17 and/or 137 of SEQ ID NO: 7 or corresponding to position 19 and/or 139 of SEQ ID NO: 21 is an amino acid substitution, preferably a substitution of leucine by phenylalanine at a position corresponding to position 137 of SEQ ID NO: 7 L137F as presented in SEQ ID NO: 8, or corresponding to position 139 of SEQ ID NO: 21 L139F as presented in SEQ ID NO: 39, and/or an amino acid substitution of threonine by isoleucine at a position corresponding to position 17 of SEQ ID NO: 7 T17I as presented in SEQ ID NO: 18 and/or an amino acid substitution of serine by isoleucine at a position corresponding to position 19 of SEQ ID NO: 21 S19I as presented in SEQ ID NO: 36.

In another preferred embodiment the alteration of said polypeptide at a position corresponding to position 15 of SEQ ID NO: 7 is a premature polypeptide termination R15*, as presented in SEQ ID NO: 15, resulting in a polypeptide with a length of 14 amino acids, corresponding to amino acids 1-14 of SEQ ID NO: 7.

In another embodiment, the alteration of said polypeptide at the position corresponding to position 137 of SEQ ID NO: 7 or corresponding to position 139 of SEQ ID NO: 21 is an amino acid substitution, such as a substitution of leucine by isoleucine, valine, histidine, lysine, methionine, threonine, tryptophan, asparagine, aspartic acid/aspartate, alanine, arginine, cysteine/cystine, glutamine, glutamic acid/glutamate, glycine, proline, serine, phenylalanine or tyrosine.

In another embodiment, the alteration of said polypeptide at the position corresponding to position 17 of SEQ ID NO: 7 is an amino acid substitution, such as a substitution of threonine by isoleucine, valine, histidine, lysine, methionine, leucine, tryptophan, asparagine, aspartic acid/aspartate, alanine, arginine, cysteine/cystine, glutamine, glutamic acid/glutamate, glycine, proline, serine, phenylalanine, or tyrosine.

In one embodiment the alteration of said polypeptide at the position corresponding to position 17 and/or 137 of SEQ ID NO: 7 is an amino acid insertion, such as an amino acid insertion of at least one amino acid selected from the list of leucine, phenylalanine, isoleucine, valine, histidine, lysine, methionine, threonine, tryptophan, asparagine, aspartic acid/aspartate, alanine, arginine, cysteine/cystine, glutamine, glutamic acid/glutamate, glycine, proline, serine, or tyrosine. The amino acid insertion can be an insertion of one, two, three, four, five, or more than five amino acids, wherein the amino acids are independently chosen from one another.

In another embodiment, the alteration of said polypeptide at the position corresponding to position 19 of SEQ ID NO: 21 is an amino acid substitution, such as a substitution of serine by isoleucine, valine, histidine, lysine, methionine, leucine, tryptophan, asparagine, aspartic acid/aspartate, alanine, arginine, cysteine/cystine, glutamine, glutamic acid/glutamate, glycine, proline, threonine, phenylalanine, or tyrosine.

In one embodiment the alteration of said polypeptide at the position corresponding to position 19 and/or 139 of SEQ ID NO: 21 is an amino acid insertion, such as an amino acid insertion of at least one amino acid selected from the list of leucine, phenylalanine, isoleucine, valine, histidine, lysine, methionine, threonine, tryptophan, asparagine, aspartic acid/aspartate, alanine, arginine, cysteine/cystine, glutamine, glutamic acid/glutamate, glycine, proline, serine, or tyrosine. The amino acid insertion can be an insertion of one, two, three, four, five, or more than five amino acids, wherein the amino acids are independently chosen from one another.

In a preferred embodiment, the fungal host cell is a yeast host cell; preferably the yeast host cell is selected from the group consisting of Candida, Hansenula, Kluyveromyces, Pichia (Komagataella), Saccharomyces, Schizosaccharomyces, and Yarrowia cell; more preferably the yeast host cell is selected from the group consisting of Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, and Yarrowia lipolytica cell, most preferably Pichia pastoris (Komagataella phaffii).

In another preferred embodiment, the fungal host cell is a filamentous fungal host cell; preferably the filamentous fungal host cell is selected from the group consisting of Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, and Trichoderma cell; more preferably the filamentous fungal host cell is selected from the group consisting of Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, and Trichoderma viride cell; even more preferably the filamentous host cell is selected from the group consisting of Aspergillus oryzae, Fusarium venenatum, and Trichoderma reesei cell; most preferably the filamentous fungal host cell is an Aspergillus niger cell.

In a preferred embodiment, the fungal host cell is an Aspergillus oryzae cell.

In yet another embodiment, the fungal host cell is a Trichoderma reesei cell.

In a further preferred embodiment, the polypeptide of interest comprises an enzyme; preferably the enzyme is selected from the group consisting of hydrolase, isomerase, ligase, lyase, oxidoreductase, or transferase; more preferably an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, alpha-glucosidase, beta-glucosidase, invertase, laccase, lipase, mannosidase, mutanase, nuclease, oxidase, pectinolytic enzyme, peroxidase, phosphodiesterase, phytase, polyphenoloxidase, proteolytic enzyme, ribonuclease, transglutaminase, xylanase, and beta-xylosidase.

In yet another preferred embodiment, the polypeptide of interest is a glucoamylase.

In another preferred embodiment, the polypeptide of interest is a lysozyme.

The host cells of the instant invention are cultivated in a nutrient medium suitable for production of the polypeptide using methods known in the art. For example, the cells may be cultivated by shake flask cultivation, or small-scale or large-scale fermentation (including continuous, batch, fed-batch, or solid-state fermentations) in laboratory or industrial fermentors in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide

The polypeptide may be recovered using methods known in the art. For example, the polypeptide may be recovered from the fermentation medium by conventional procedures including, but not limited to, collection, centrifugation, filtration, extraction, spray-drying, evaporation, or precipitation. In one aspect, a whole fermentation broth comprising the polypeptide is recovered.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

### Nucleic Acid Constructs

In a third aspect, the present invention also relates to a nucleic acid construct comprising a heterologous promoter operably linked to a polynucleotide encoding a polypeptide having a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100% to SEQ ID NO: 7 and comprising an alteration at a position corresponding to position 15, 17 and/or 137 of SEQ ID NO: 7, or corresponding to position 19 and/or 139 of SEQ ID NO: 21.

In a preferred embodiment the alteration at the position corresponding to position 17 and/or 137 of SEQ ID NO: 7, or corresponding to position 19 and/or 139 of SEQ ID NO: 21 is a substitution; preferably a substitution of leucine by phenylalanine at a position corresponding to position 137 of SEQ ID NO: 7 L137F according to SEQ ID NO 8 or corresponding to position 139 of SEQ ID NO: 139 L139F according to SEQ ID NO: 39; and/or an amino acid substitution of threonine by isoleucine at a position corresponding to position 17 of SEQ ID NO: 7 T17I according to SEQ ID NO: 18, and/or an amino acid substitution of serine by isoleucine at a position corresponding to position 19 of SEQ ID NO: 21 S19I according to SEQ ID NO: 36.

In one preferred embodiment the alteration at the position corresponding to position 15 of SEQ ID NO: 7 is a premature polypeptide termination R15*. This alteration leads to a polypeptide with a length of 14 amino acids, corresponding to amino acids 1-14 of SEQ ID NO: 7.

In another embodiment, the alteration at the position corresponding to position 17 and/or 137 of SEQ ID NO: 7 or corresponding to position 19 and/or 139 of SEQ ID NO: 21 comprises or consists of an alteration, preferably a substitution, wherein the variant has a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, to the amino acid sequence of SEQ ID NO: 7 or SEQ ID NO: 21. The amino acid at a position corresponding to position 137 of SEQ ID NO: 7 or corresponding to position 139 of SEQ ID NO: 21 is substituted with Ala, Arg, Asn, Asp, Cys, Gln, Glu, Gly, His, lie, Leu, Lys, Met, Pro, Ser, Thr, Trp, Tyr, or Val, preferably with Phe. Alternatively or additionally, the amino acid at a position corresponding to position 17 of SEQ ID NO: 7 or corresponding to position 19 of SEQ ID NO: 21 is substituted with Ala, Arg, Asn, Asp, Cys, Gln, Glu, Gly, His, Phe, Leu, Lys, Met, Pro, Ser, Thr, Trp, Tyr, or Val, preferably with Ile. In a preferred embodiment, the variant comprises or consists of the substitution L137F as shown in SEQ ID NO: 8. In a preferred embodiment, the variant comprises or consists of the substitution L139F as shown in SEQ ID NO: 39. In another preferred embodiment, the variant additionally or alternatively comprises or consists of the substitution T17I as shown in SEQ ID NO: 18 or of the substitution S17I as shown in SEQ ID NO: 36.

In another embodiment, the alteration of said polypeptide at the position corresponding to position 137 of SEQ ID NO: 7 or corresponding to position 139 of SEQ ID NO: 21 is an amino acid substitution, such as a substitution of leucine by isoleucine, valine, histidine, lysine, methionine, threonine, tryptophan, asparagine, aspartic acid/aspartate, alanine, arginine, cysteine/cystine, glutamine, glutamic acid/glutamate, glycine, proline, serine, or tyrosine.

In another embodiment, the alteration of said polypeptide at the position corresponding to position 17 of SEQ ID NO: 7 is an amino acid substitution, such as a substitution of threonine by isoleucine, valine, histidine, lysine, methionine, leucine, tryptophan, asparagine, aspartic acid/aspartate, alanine, arginine, cysteine/cystine, glutamine, glutamic acid/glutamate, glycine, proline, serine, or tyrosine.

In another embodiment, the alteration of said polypeptide at the position corresponding to position 19 of SEQ ID NO: 21 is an amino acid substitution, such as a substitution of serine by isoleucine, valine, histidine, lysine, methionine, leucine, tryptophan, asparagine, aspartic acid/aspartate, alanine, arginine, cysteine/cystine, glutamine, glutamic acid/glutamate, glycine, proline, threonine, or tyrosine.

In one embodiment the alteration of said polypeptide at the position corresponding to position 17 and/or 137 of SEQ ID NO: 7 is an amino acid insertion, such as an amino acid insertion of at least one amino acid selected from the list of leucine, phenylalanine, isoleucine, valine, histidine, lysine, methionine, threonine, tryptophan, asparagine, aspartic acid/aspartate, alanine, arginine, cysteine/cystine, glutamine, glutamic acid/glutamate, glycine, proline, serine, or tyrosine. In another embodiment the alteration of said polypeptide at the position corresponding to position 19 and/or 139 of SEQ ID NO: 21 is an amino acid insertion, such as an amino acid insertion of at least one amino acid selected from the list of leucine, phenylalanine, isoleucine, valine, histidine, lysine, methionine, threonine, tryptophan, asparagine, aspartic acid/aspartate, alanine, arginine, cysteine/cystine, glutamine, glutamic acid/glutamate, glycine, proline, serine, or tyrosine. The amino acid insertion can be an insertion of one, two, three, four, five, or more than five amino acids, wherein the amino acids are independently chosen from one another.

In a preferred embodiment of the third aspect, the polynucleotide encoding the polypeptide has a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100% to SEQ ID NO: 3, SEQ ID NO: 4, or SEQ ID NO: 28.

In another preferred embodiment, the polynucleotide encoding the polypeptide has a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100% to SEQ ID NO: 3 and comprises at least one nucleotide alteration at a position corresponding to position 495, 496 and/or 497 of SEQ ID NO: 3.

In another preferred embodiment, the polynucleotide encoding the polypeptide has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100% to SEQ ID NO: 28 and comprises at least one nucleotide alteration at a position corresponding to position 656, 657 and/or 658 of SEQ ID NO: 28.

In a preferred embodiment the at least one nucleotide alteration is a substitution; preferably the nucleotide(s) at a position corresponding to position 495, 496, and/or 497 of SEQ ID NO: 3 or corresponding to position 656, 657 and/or 658 of SEQ ID NO: 28 is/are independently from another substituted with adenine (A), thymine (T), guanine (G) and/or cytosine (C).

In another preferred embodiment, the at least one nucleotide alteration comprises or consists of a substitution of cytosine with thymine at a position corresponding to position 495 of SEQ ID NO: 3, C495T, as shown in SEQ ID NO: 5. Alternatively, the at least one nucleotide alteration comprises or consists of a substitution of cytosine with thymine at a position corresponding to position 495 of SEQ ID NO: 3, C495T, and of a substitution of thymine with cytosine at a position corresponding to position 497 of SEQ ID NO: 3.

In one embodiment, one, two or three of the nucleotides corresponding to position 495, 496 and/or 497 of SEQ ID NO: 3 or corresponding to position 656, 657 and/or 658 of SEQ ID NO: 28 are independently from another substituted with adenine (A), thymine (T), guanine (G) or cytosine (C).

In another embodiment, one, two or three of the nucleotides corresponding to position 495, 496 and/or 497 of SEQ ID NO: 3 or corresponding to position 656, 657 and/or 658 of SEQ ID NO: 28 are, independently from another, deleted.

In another preferred embodiment, the polynucleotide encoding the polypeptide has a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100% to SEQ ID NO: 4 and comprises at least one nucleotide alteration at a position corresponding to position 409, 410 and/or 411 of SEQ ID NO: 4.

In one embodiment the at least one nucleotide alteration is a substitution; preferably the nucleotide(s) at a position corresponding to position 409, 410, and/or 411 of SEQ ID NO: 4 is/are independently from another substituted with adenine (A), thymine (T), guanine (G) and/or cytosine (C).

In a preferred embodiment, the at least one nucleotide alteration comprises or consists of a substitution of cytosine with thymine at a position corresponding to position 409 of SEQ ID NO: 4, C409T, as shown in SEQ ID NO: 6. Alternatively, the at least one nucleotide alteration comprises or consists of a substitution of cytosine with thymine at a position corresponding to position 495 of SEQ ID NO: 4, C409T, and of a substitution of thymine with cytosine at a position corresponding to position 411 of SEQ ID NO: 4.

In one embodiment, one, two or three of the nucleotides corresponding to position 409, 410 and/or 411 of SEQ ID NO: 4 are independently from another substituted with adenine (A), thymine (T), guanine (G) or cytosine (C).

In another embodiment, one, two or three of the nucleotides corresponding to position 409, 410 and/or 411 of SEQ ID NO: 4 are, independently from another, deleted.

In another preferred embodiment, the polynucleotide encoding the polypeptide has a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100% to SEQ ID NO: 3 or SEQ ID NO: 28 and comprises at least one nucleotide alteration at a position corresponding to position 49, 50 and/or 51 of SEQ ID NO: 3 or corresponding to position 55, 56 and/or 57 of SEQ ID NO: 28.

In a preferred embodiment the at least one nucleotide alteration is a substitution; preferably the nucleotide(s) at a position corresponding to position 49, 50, and/or 51 of SEQ ID NO: 3 or corresponding to position 55, 56 and/or 57 of SEQ ID NO: 28 is/are independently from another substituted with adenine (A), thymine (T), guanine (G) and/or cytosine (C).

In another preferred embodiment, the at least one nucleotide alteration comprises or consists of a substitution of cytosine by thymine at a position corresponding to position 50 of SEQ ID NO: 3, C50T, as shown in SEQ ID NO: 16. Alternatively, the at least one nucleotide alteration comprises or consists of a substitution of cytosine by thymine at a position corresponding to position 50 of SEQ ID NO: 3, C50T, and of a substitution of adenine with thymine at a position corresponding to position 51 of SEQ ID NO: 3. Alternatively, the at least one nucleotide alteration comprises or consists of a substitution of cytosine by thymine at a position corresponding to position 50 of SEQ ID NO: 3, C50T, and of a substitution of adenine with cytosine at a position corresponding to position 51 of SEQ ID NO: 3.

In one embodiment, one, two or three of the nucleotides corresponding to position 49, 50 and/or 51 of SEQ ID NO: 3 or corresponding to position 55, 56 and/or 57 of SEQ ID NO: 28 are independently from another substituted with adenine (A), thymine (T), guanine (G) or cytosine (C).

In another embodiment, one, two or three of the nucleotides corresponding to position 49, 50 and/or 51 of SEQ ID NO: 3 or corresponding to position 55, 56 and/or 57 of SEQ ID NO: 28 are, independently from another, deleted.

In another preferred embodiment, the polynucleotide encoding the polypeptide has a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100% to SEQ ID NO: 4 and comprises at least one nucleotide alteration at a position corresponding to position 49, 50 and/or 51 of SEQ ID NO: 4.

In one embodiment the at least one nucleotide alteration is a substitution; preferably the nucleotide(s) at a position corresponding to position 49, 50, and/or 51 of SEQ ID NO: 4 is/are independently from another substituted with adenine (A), thymine (T), guanine (G) and/or cytosine (C).

In a preferred embodiment, the at least one nucleotide alteration comprises or consists of a substitution of cytosine by thymine at a position corresponding to position 50 of SEQ ID NO: 4, C50T, as shown in SEQ ID NO: 17. Alternatively, the at least one nucleotide alteration comprises or consists of a substitution of cytosine by thymine at a position corresponding to position 50 of SEQ ID NO: 4, C50T, and of a substitution of adenine with thymine at a position corresponding to position 51 of SEQ ID NO: 4. Alternatively, the at least one nucleotide alteration comprises or consists of a substitution of cytosine by thymine at a position corresponding to position 50 of SEQ ID NO: 4, C50T, and of a substitution of adenine with cytosine at a position corresponding to position 51 of SEQ ID NO: 4.

In one embodiment, one, two or three of the nucleotides corresponding to position 49, 50 and/or 51 of SEQ ID NO: 4 are independently from another substituted with adenine (A), thymine (T), guanine (G) or cytosine (C).

In another embodiment, one, two or three of the nucleotides corresponding to position 49, 50 and/or 51 of SEQ ID NO: 4 are, independently from another, deleted.

In a preferred embodiment the polypeptide with the T17I mutation is the polypeptide with SEQ ID NO: 18.

In another preferred embodiment, the at least one nucleotide alteration leads to a missense mutation and/or a premature stop codon, wherein the polynucleotide encoding the polypeptide has a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100% to SEQ ID NO: 3 and comprises at least one nucleotide alteration at a position corresponding to position 43, 44 and/or 45 of SEQ ID NO: 3.

In a preferred embodiment the at least one nucleotide alteration is a substitution; preferably the nucleotide(s) at a position corresponding to position 43, 44, and/or 45 of SEQ ID NO: 3 is/are independently from another substituted with adenine (A), thymine (T), guanine (G) and/or cytosine (C).

In another preferred embodiment, the at least one nucleotide alteration comprises or consists of a substitution of cytosine by thymine at a position corresponding to position 43 of SEQ ID NO: 3, C43T, as shown in SEQ ID NO: 13, leading to a premature stop codon at the amino acid position corresponding to position 15 of SEQ ID NO: 7. Alternatively, the at least one nucleotide alteration comprises or consists of a substitution of cytosine by thymine at a position corresponding to position 43 of SEQ ID NO: 3, C43T, and of a substitution of guanine by adenine at a position corresponding to position 44 of SEQ ID NO: 3, G44A. Alternatively, the at least one nucleotide alteration comprises or consists of a substitution of cytosine by thymine at a position corresponding to position 43 of SEQ ID NO: 3, C43T, and of a substitution of guanine by adenine at a position corresponding to position 44 of SEQ ID NO: 3, G44A, and of a substitution of adenine by guanine at a position corresponding to position 45 of SEQ ID NO: 3, A45G.

In one embodiment, one, two or three of the nucleotides corresponding to position 43, 44 and/or 45 of SEQ ID NO: 3 are independently from another substituted with adenine (A), thymine (T), guanine (G) or cytosine (C).

In another embodiment, one, two or three of the nucleotides corresponding to position 43, 44 and/or 45 of SEQ ID NO: 3 are, independently from another, deleted.

In another preferred embodiment, the polynucleotide encoding the polypeptide has a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100% to SEQ ID NO: 4 and comprises at least one nucleotide alteration at a position corresponding to position 43, 44 and/or 45 of SEQ ID NO: 4.

In one embodiment the at least one nucleotide alteration is a substitution; preferably the nucleotide(s) at a position corresponding to position 43, 44, and/or 45 of SEQ ID NO: 4 is/are independently from another substituted with adenine (A), thymine (T), guanine (G) and/or cytosine (C).

In a preferred embodiment, the at least one nucleotide alteration comprises or consists of a substitution of cytosine by thymine at a position corresponding to position 43 of SEQ ID NO: 4, C43T, as shown in SEQ ID NO: 14. Alternatively, the at least one nucleotide alteration comprises or consists of a substitution of cytosine by thymine at a position corresponding to position 43 of SEQ ID NO: 4, C43T, and of a substitution of guanine by adenine at a position corresponding to position 44 of SEQ ID NO: 4, G44A. Alternatively, the at least one nucleotide alteration comprises or consists of a substitution of cytosine by thymine at a position corresponding to position 43 of SEQ ID NO: 4, C43T, and of a substitution of guanine by adenine at a position corresponding to position 44 of SEQ ID NO: 4, G44A, and of a substitution of adenine by guanine at a position corresponding to position 45 of SEQ ID NO: 4, A45G.

In one embodiment, one, two or three of the nucleotides corresponding to position 43, 44 and/or 45 of SEQ ID NO: 4 are independently from another substituted with adenine (A), thymine (T), guanine (G) or cytosine (C).

In another embodiment, one, two or three of the nucleotides corresponding to position 43, 44 and/or 45 of SEQ ID NO: 4 are, independently from another, deleted.

In a preferred embodiment the polypeptide with the premature polypeptide termination is the polypeptide with SEQ ID NO: 15.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the polynucleotide of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, J. Bacteriol. 177: 3465-3471).

The control sequence may also be a leader, a non-translated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is heterologous to the coding sequence. A heterologous signal peptide coding sequence may be required where the coding sequence does not naturally contain a signal peptide coding sequence. Alternatively, a heterologous signal peptide coding sequence may simply replace the natural signal peptide coding sequence to enhance secretion of the polypeptide. However, any signal peptide coding sequence that directs the expressed polypeptide into the secretory pathway of a host cell may be used.

Effective signal peptide coding sequences for filamentous fungal host cells are the signal peptide coding sequences obtained from the genes for Aspergillus niger neutral amylase, Aspergillus nigerglucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

### Expression Vectors

In a fourth aspect, the present invention also relates to an expression vector comprising a nucleic acid construct according to the third aspect of the present invention.

In a preferred embodiment said nucleic acid construct comprising a heterologous promoter operably linked to a polynucleotide encoding a polypeptide having a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100% to SEQ ID NO: 7 or SEQ ID NO: 21 and comprising an alteration at a position corresponding to position 15, 17 and/or 137 of SEQ ID NO: 7 or corresponding to position 19 and/or 139 of SEQ ID NO: 21.

In a preferred embodiment the alteration at the position corresponding to position 137 of SEQ ID NO: 7 is a substitution; preferably a substitution of leucine for phenylalanine, L137F according to SEQ ID NO 8.

In another preferred embodiment the alteration at the position corresponding to position 17 of SEQ ID NO: 7 is a substitution; preferably a substitution of threonine for isoleucine, T17I according to SEQ ID NO 18.

In another preferred embodiment the alteration at the position corresponding to position 15 of SEQ ID NO: 7 is a premature polypeptide termination R15* according to SEQ ID NO: 15.

In a preferred embodiment the alteration at the position corresponding to position 139 of SEQ ID NO: 21 is a substitution; preferably a substitution of leucine for phenylalanine, L139F according to SEQ ID NO 39.

In another preferred embodiment the alteration at the position corresponding to position 19 of SEQ ID NO: 21 is a substitution; preferably a substitution of serine for isoleucine, S19I according to SEQ ID NO 36.

In another embodiment, the alteration at the position corresponding to position 17 and/or 137 of SEQ ID NO: 7 comprises or consists of an alteration, preferably a substitution, wherein the variant has a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, to the amino acid sequence of SEQ ID NO: 7. In one embodiment, the amino acid at a position corresponding to position 137 of SEQ ID NO: 7 is substituted with Ala, Arg, Asn, Asp, Cys, Gln, Glu, Gly, His, lie, Leu, Lys, Met, Ser, Pro, Ser, Thr, Trp, Tyr, or Val, preferably with Phe. In another preferred embodiment, the alteration comprises or consists of the substitution L137F of SEQ ID NO: 8. In another embodiment, the amino acid at a position corresponding to position 17 of SEQ ID NO: 7 is substituted with Ala, Arg, Asn, Asp, Cys, Gln, Glu, Gly, His, Phe, Leu, Lys, Met, Ser, Pro, Ser, Thr, Trp, Tyr, or Val, preferably with Ile. In another preferred embodiment, the alteration comprises or consists of the substitution T17I of SEQ ID NO: 18.

In another embodiment, the alteration at the position corresponding to position 19 and/or 139 of SEQ ID NO: 21 comprises or consists of an alteration, preferably a substitution, wherein the variant has a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, to the amino acid sequence of SEQ ID NO: 21. In one embodiment, the amino acid at a position corresponding to position 139 of SEQ ID NO: 21 is substituted with Ala, Arg, Asn, Asp, Cys, Gln, Glu, Gly, His, lie, Leu, Lys, Met, Ser, Pro, Ser, Thr, Trp, Tyr, or Val, preferably with Phe. In another preferred embodiment, the alteration comprises or consists of the substitution L139F of SEQ ID NO: 39. In another embodiment, the amino acid at a position corresponding to position 19 of SEQ ID NO: 21 is substituted with Ala, Arg, Asn, Asp, Cys, Gln, Glu, Gly, His, Phe, Leu, Lys, Met, Ser, Pro, Ser, Thr, Trp, Tyr, or Val, preferably with Ile. In another preferred embodiment, the alteration comprises or consists of the substitution S19I of SEQ ID NO: 36.

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a mini-chromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosylaminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is a hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMB1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

Polypeptides Having Man₅GlcNAc₂-PP-Dol Alpha-1,3-Mannosyltransferase Activity

In some embodiments, the present invention relates to isolated or purified polypeptides having a sequence identity of at least 50%, e.g., at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide of SEQ ID NO: 7 or SEQ ID NO: 21. The polypeptide of SEQ ID NO: 7 or SEQ ID NO: 21 has Man₅GlcNAc₂-PP-Dol alpha-1,3-mannosyltransferase activity. In one embodiment, the polypeptides differ by up to 10 amino acids, e.g., 1, 2, 3, 4, 5, 6, 7, 8, 9, or 10 amino acids, from the mature polypeptide of SEQ ID NO: 7 or SEQ ID NO: 21. More preferably, the polypeptide differs by 1 amino acid from the mature polypeptide of SEQ ID NO: 7 or SEQ ID NO: 21.

The mature polypeptide preferably comprises, consists essentially of, or consists of the amino acid sequence of SEQ ID NO: 7 or SEQ ID NO: 21 or the mature polypeptide thereof; or is a fragment thereof having altered, reduced or eliminated Man₅GlcNAc₂-PP-Dol alpha-1,3-mannosyltransferase activity. In a further embodiment, the mature polypeptide is SEQ ID NO: 8 or SEQ ID NO: 39 and has altered, reduced or eliminated Man₅GlcNAc₂-PP-Dol alpha-1,3-mannosyltransferase activity compared to SEQ ID NO: 7 or SEQ ID NO: 21, respectively. In yet another embodiment, the mature polypeptide is SEQ ID NO: 8 or SEQ ID NO: 39 having altered, reduced or eliminated Man₅GlcNAc₂-PP-Dol alpha-1,3-mannosyltransferase activity when compared to the polypeptide of SEQ ID NO: 7 or SEQ ID NO: 21, respectively, such as a decreased activity of 90%, 80%, 70%, 60%, 50%, 40%, 30%, 20%, 10%, 5%, 1%, 0,1%, or 0,01% or lower, or substantially 0% when compared to the activity of SEQ ID NO: 7 or SEQ ID NO: 21, respectively. In another embodiment, the mature polypeptide is SEQ ID NO: 8 or SEQ ID NO: 39 having a decreased Man₅GlcNAc₂-PP-Dol alpha-1,3-mannosyltransferase activity, such as a decrease of 95%, 97%, 98% or 99% of activity when compared to the activity of the polypeptide of SEQ ID NO: 7.

In another embodiment, the mature polypeptide is SEQ ID NO: 15 having reduced or eliminated Man₅GlcNAc₂-PP-Dol alpha-1,3-mannosyltransferase activity when compared to SEQ ID NO: 7. In yet another embodiment, the mature polypeptide is SEQ ID NO: 15 having altered, reduced or eliminated Man₅GlcNAc₂-PP-Dol alpha-1,3-mannosyltransferase activity when compared to the polypeptide of SEQ ID NO: 7, such as a decreased activity of 90%, 80%, 70%, 60%, 50%, 40%, 30%, 20%, 10%, 5%, 1%, 0,1%, or 0,01% or lower, or substantially 0% when compared to the activity of SEQ ID NO: 7.

In a further embodiment, the mature polypeptide is SEQ ID NO: 18 or SEQ ID NO: 36 having altered, reduced or eliminated Man₅GlcNAc₂-PP-Dol alpha-1,3-mannosyltransferase activity when compared to SEQ ID NO: 7 or SEQ ID NO: 21, respectively. In yet another embodiment, the mature polypeptide is SEQ ID NO: 18 or SEQ ID NO: 36 having reduced Man₅GlcNAc₂-PP-Dol alpha-1,3-mannosyltransferase activity when compared to the polypeptide of SEQ ID NO: 7 or SEQ ID NO: 21, respectively, such as a decreased activity of 90%, 80%, 70%, 60%, 50%, 40%, 30%, 20% or 10%, or lower than 10% when compared to the activity of SEQ ID NO: 7 or SEQ ID NO: 21, respectively. In another embodiment, the mature polypeptide is SEQ ID NO: 18 or SEQ ID NO: 36 having a decreased Man₅GlcNAc₂-PP-Dol alpha-1,3-mannosyltransferase activity, such as a decrease of 95%, 97%, 98% or 99% of activity when compared to the activity of the polypeptide of SEQ ID NO: 7 or SEQ ID NO: 21, respectively.

In some embodiments, the present invention relates to isolated or purified polypeptides having Man₅GlcNAc₂-PP-Dol alpha-1,3-mannosyltransferase activity encoded by polynucleotides that hybridize under medium stringency conditions, medium-high stringency conditions, high stringency conditions, or very high stringency conditions with the full-length complement of the mature polypeptide coding sequence of SEQ ID NO: 3 or the cDNA thereof (SEQ ID NO: 4), or of the mature polypeptide coding sequence of SEQ ID NO: 5 or the cDNA thereof (SEQ ID NO: 6), or the mature polypeptide coding sequence of SEQ ID NO: 13 or the cDNA thereof (SEQ ID NO: 14), or the mature polypeptide coding sequence of SEQ ID NO: 16 or the cDNA thereof (SEQ ID NO: 17), or the mature polypeptide coding sequence of SEQ ID NO: 28 or the cDNA thereof (SEQ ID NO: 40), or the mature polypeptide coding sequence of SEQ ID NO: 34 or the cDNA thereof (SEQ ID NO: 35), or the mature polypeptide coding sequence of SEQ ID NO: 37 or the cDNA thereof (SEQ ID NO: 38) (Sambrook et al., 1989, Molecular Cloning, A Laboratory Manual, 2d edition, Cold Spring Harbor, New York).

The polynucleotide of SEQ ID NO: 3 or SEQ ID NO: 28 or a subsequence thereof, as well as the mature polypeptide of SEQ ID NO: 7 or SEQ ID NO: 21 or a fragment thereof, may be used to design nucleic acid probes to identify and clone DNA encoding polypeptides having altered, reduced or eliminated Man₅GlcNAc₂-PP-Dol alpha-1,3-mannosyltransferase activity from strains of different genera or species according to methods well known in the art. Such probes can be used for hybridization with the genomic DNA or cDNA of a cell of interest, following standard Southern blotting procedures, in order to identify and isolate the corresponding gene therein. Such probes can be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other strains may be screened for DNA that hybridizes with the probes described above and encodes a polypeptide having Man₅GlcNAc₂-PP-Dol alpha-1,3-mannosyltransferase activity. Genomic or other DNA from such other strains may be separated by agarose or polyacrylamide gel electrophoresis, or other separation techniques. DNA from the libraries or the separated DNA may be transferred to and immobilized on nitrocellulose or another suitable carrier material. In order to identify a clone or DNA that hybridizes with SEQ ID NO: 3, 4, 5, 6, 13, 14, 16,17, 28, 34, 35, 37, 38 or 40 or a subsequence thereof, the carrier material is used in a Southern blot.

For purposes of the present invention, hybridization indicates that the polynucleotides hybridize to a labeled nucleic acid probe corresponding to (i) SEQ ID NO: 3, 5, 13, 16, 28, 34 or 37; (ii) the mature polypeptide coding sequence of SEQ ID NO: 3, 5, 13, 16, 28, 34, or 37; (iii) the cDNA sequence thereof; (iv) the full-length complement thereof; or (v) a subsequence thereof; under medium to very high stringency conditions. Molecules to which the nucleic acid probe hybridizes under these conditions can be detected using, for example, X-ray film or any other detection means known in the art.

In some embodiments, the nucleic acid probe is nucleotides 1 to 400, nucleotides 400 to 800, nucleotides 800 to 1200, or nucleotides 1000 to 1400 of SEQ ID NO: 3, 5, 13,16, 28, 34, or 37. In another aspect, the nucleic acid probe is a polynucleotide that encodes the mature polypeptide of SEQ ID NO: 7, 8, 15,18, 21, 36 or 39; or a fragment thereof. In another aspect, the nucleic acid probe is SEQ ID NO: 3 or the cDNA sequence thereof, or SEQ ID NO: 5 or the cDNA sequence thereof, or SEQ ID NO: 28 or the cDNA sequence thereof, or SEQ ID NO: 34 or the cDNA sequence thereof, or SEQ ID NO: 37 or the cDNA sequence thereof. In another aspect, the nucleic acid probe is the polynucleotide contained in plasmid plhar531, wherein the polynucleotide encodes a polypeptide or a fraction of a polypeptide having altered, reduced or eliminated Man₅GlcNAc₂-PP-Dol alpha-1,3-mannosyltransferase activity.

In some embodiments, the present invention relates to isolated polypeptides encoded by polynucleotides having a sequence identity of at least 50%, e.g., at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 3 or the cDNA sequence thereof (SEQ ID NO: 4), or to the mature polypeptide coding sequence of SEQ ID NO: 5 or the cDNA sequence thereof (SEQ ID NO: 6), or to the mature polypeptide coding sequence of SEQ ID NO: 13 or the cDNA sequence thereof (SEQ ID NO: 14), or to the mature polypeptide coding sequence of SEQ ID NO: 16 or the cDNA sequence thereof (SEQ ID NO: 17), or the mature polypeptide coding sequence of SEQ ID NO: 28 or the cDNA sequence thereof (SEQ ID NO: 40), or the mature polypeptide coding sequence of SEQ ID NO: 34 or the cDNA sequence thereof (SEQ ID NO: 35), or the mature polypeptide coding sequence of SEQ ID NO: 37 or the cDNA sequence thereof (SEQ ID NO: 38).

In a preferred embodiment, the polypeptide has a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100%, to SEQ ID NO: 7 or SEQ ID NO: 21 and the polypeptide comprises an alteration, preferably a substitution, deletion or insertion, of an amino acid at a position corresponding to position 15, 17 and/or 137 of SEQ ID NO: 7 or corresponding to position 19 and/or 139 of SEQ ID NO: 21.

In another preferred embodiment, the polypeptide has a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100%, to SEQ ID NO: 7 and the polypeptide comprises an amino acid substitution at the position corresponding to position 17 and/or 137 of SEQ ID NO: 7, preferably a substitution of leucine by phenylalanine, L137F (SEQ ID NO: 8) and/or a substitution of threonine by isoleucine, T17I (SEQ ID NO: 18).

In another preferred embodiment, the polypeptide has a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100%, to SEQ ID NO: 21 and the polypeptide comprises an amino acid substitution at the position corresponding to position 19 and/or 139 of SEQ ID NO: 21, preferably a substitution of leucine by phenylalanine, L139F (SEQ ID NO: 39) and/or a substitution of threonine by isoleucine, S19I (SEQ ID NO: 36).

The polynucleotide encoding the polypeptide preferably comprises, consists essentially of, or consists of nucleotides 1 to 1400 of SEQ ID NO: 5, SEQ ID NO: 6, SEQ ID NO: 16, SEQ ID NO: 17, SEQ ID NO: 34, SEQ ID NO: 35, SEQ ID NO: 37 or SEQ ID NO:38.

In one embodiment the polynucleotide encoding the polypeptide comprises a premature polypeptide termination R15* and comprises, consists essentially of, or consists of nucleotides 43-45 of SEQ ID NO: 13 or SEQ ID NO: 14.

In some embodiments, the present invention relates to a polypeptide derived from a mature polypeptide of SEQ ID NO: 7 or SEQ ID NO: 21 by substitution, deletion or addition of one or several amino acids in the mature polypeptide of SEQ ID NO: 7 or SEQ ID NO: 21. In some embodiments, the present invention relates to variants of the mature polypeptide of SEQ ID NO: 7 or SEQ ID NO: 21 comprising a substitution, deletion, and/or insertion at one or more (e.g., several) positions. In one aspect, the number of amino acid substitutions, deletions and/or insertions introduced into the mature polypeptide of SEQ ID NO: 7 or SEQ ID NO: 21 is up to 10, e.g., 1, 2, 3, 4, 5, 6, 7, 8, 9, or 10. In one embodiment, the polypeptide has an N-terminal extension and/or C-terminal extension of 1-10 amino acids, e.g., 1, 2, 3, 4, 5, 6, 7, 8, 9, or 10 amino acids. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant molecules are tested for Man₅GlcNAc₂-PP-Dol alpha-1,3-mannosyltransferase activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide. Essential amino acids in the sequence of amino acids 1 to 413 of SEQ ID NO: 7 are located at positions 61-63 (R61, D62 and Y63), such as at positions 62-63 (D62 and Y63), and at position 62 of SEQ ID NO: 7 (D62).

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7:127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

In some embodiments, the polypeptide is a fragment containing at least 250 amino acid residues (e.g., amino acids 100 to 350 of the mature polypeptide of SEQ ID NO: 8), at least 150 amino acid residues (e.g., amino acids 150 to 300 of the mature polypeptide of SEQ ID NO: 8), or at least 100 amino acid residues (e.g., amino acids 300 to 400 of the mature polypeptide of SEQ ID NO: 8).

In some embodiments, the polypeptide is a fragment containing at least 250 amino acid residues (e.g., amino acids 100 to 350 of the mature polypeptide of SEQ ID NO: 18), at least 150 amino acid residues (e.g., amino acids 150 to 300 of the mature polypeptide of SEQ ID NO: 18), or at least 100 amino acid residues (e.g., amino acids 300 to 400 of the mature polypeptide of SEQ ID NO: 18).

In some embodiments, the polypeptide is a fragment containing at least 250 amino acid residues (e.g., amino acids 100 to 350 of the mature polypeptide of SEQ ID NO: 36), at least 150 amino acid residues (e.g., amino acids 150 to 300 of the mature polypeptide of SEQ ID NO: 36), or at least 100 amino acid residues (e.g., amino acids 300 to 400 of the mature polypeptide of SEQ ID NO: 36).

In some embodiments, the polypeptide is a fragment containing at least 250 amino acid residues (e.g., amino acids 100 to 350 of the mature polypeptide of SEQ ID NO: 39), at least 150 amino acid residues (e.g., amino acids 150 to 300 of the mature polypeptide of SEQ ID NO: 39), or at least 100 amino acid residues (e.g., amino acids 300 to 400 of the mature polypeptide of SEQ ID NO: 39).

Sources of Polypeptides Having Man₅GlcNAc₂-PP-Dol alpha-1,3-mannosyltransferase Activity

A polypeptide having altered, reduced or eliminated Man₅GlcNAc₂-PP-Dol alpha-1,3-mannosyltransferase activity of the present invention may be obtained from microorganisms of any genus. For purposes of the present invention, the term “obtained from” as used herein in connection with a given source shall mean that the polypeptide encoded by a polynucleotide is produced by the source or by a strain in which the polynucleotide from the source has been inserted.

In one aspect, the polypeptide is a polypeptide obtained from an Aspergillus, e.g., a polypeptide obtained from Aspergillus niger.

In another aspect, the polypeptide is a polypeptide obtained from an Trichoderma, e.g., a polypeptide obtained from Trichoderma reesei.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen und Zellkulturen GmbH (DSMZ), Centraalbureau Voor Schimmelcultures (CBS), and Agricultural Research Service Patent Culture Collection, Northern Regional Research Center (NRRL).

The polypeptides may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding the polypeptide may then be obtained by similarly screening a genomic DNA or cDNA library of another microorganism or mixed DNA sample. Once a polynucleotide encoding a polypeptide has been detected with the probe(s), the polynucleotide can be isolated or cloned by utilizing techniques that are known to those of ordinary skill in the art (see, e.g., Sambrook et al., 1989, supra).

### Polynucleotides

The present invention also relates to isolated polynucleotides encoding a polypeptide of the present invention, as described herein.

The techniques used to isolate or clone a polynucleotide are known in the art and include isolation from genomic DNA or cDNA, or a combination thereof. The cloning of the polynucleotides from genomic DNA can be affected, e.g., by using the polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used. The polynucleotides may be cloned from a strain of Aspergillus, or a related organism and thus, for example, may be a species variant of the polypeptide encoding region of the polynucleotide.

Modification of a polynucleotide encoding a polypeptide of the present invention may be necessary for synthesizing polypeptides substantially similar to the polypeptide. The term “substantially similar” to the polypeptide refers to non-naturally occurring forms of the polypeptide. These polypeptides may differ in some engineered way from the polypeptide isolated from its native source, e.g., variants that differ in specific activity, thermostability, pH optimum, or the like. The variants may be constructed on the basis of the polynucleotide presented as the mature polypeptide coding sequence of SEQ ID NO: 3 or SEQ ID NO: 28 or the cDNA sequences thereof (SEQ ID NO: 4 or SEQ ID NO: 40 respectively), e.g., a subsequence thereof, and/or by introduction of nucleotide substitutions that do not result in a change in the amino acid sequence of the polypeptide, but which correspond to the codon usage of the host organism intended for production of the enzyme, or by introduction of nucleotide substitutions that may give rise to a different amino acid sequence. For a general description of nucleotide substitution, see, e.g., Ford et al., 1991, Protein Expression and Purification 2: 95-107.

In a fifth aspect, the present invention also relates to a polynucleotide encoding a polypeptide having a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100% to SEQ ID NO: 7 or SEQ ID NO: 21 and comprising an alteration at a position corresponding to position 15, 17 and/or 137 of SEQ ID NO: 7 or corresponding to position 19 and/or 139 of SEQ ID NO: 21.

In a preferred embodiment the alteration at the position corresponding to position 137 of SEQ ID NO: 7 is a substitution; preferably a substitution of leucine for phenylalanine, L137F according to SEQ ID NO 8.

In a preferred embodiment the alteration at the position corresponding to position 17 of SEQ ID NO: 7 is a substitution; preferably a substitution of threonine for isoleucine, T17I according to SEQ ID NO 18.

In a preferred embodiment the alteration at the position corresponding to position 139 of SEQ ID NO: 21 is a substitution; preferably a substitution of leucine for phenylalanine, L139F according to SEQ ID NO 39.

In a preferred embodiment the alteration at the position corresponding to position 19 of SEQ ID NO: 21 is a substitution; preferably a substitution of serine for isoleucine, S19I according to SEQ ID NO 36.

In another preferred embodiment the alteration at the position corresponding to position 15 of SEQ ID NO: 7 is a premature polypeptide termination R15* according to SEQ ID NO 15.

In another embodiment, the alteration at the position corresponding to position 17 and/or 137 of SEQ ID NO: 7 or corresponding to position 19 and/or 139 of SEQ ID NO: 21 comprises or consists of an alteration, preferably a substitution, wherein the variant has a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, to the amino acid sequence of SEQ ID NO: 7 or SEQ ID NO: 21. The amino acid at a position corresponding to position 137 of SEQ ID NO: 7 or corresponding to position 139 of SEQ ID NO: 21 is substituted with Ala, Arg, Asn, Asp, Cys, Gln, Glu, Gly, His, lie, Leu, Lys, Met, Pro, Ser, Thr, Trp, Tyr, or Val, preferably with Phe. In another embodiment, the variant comprises or consists of the substitution L137F as shown in SEQ ID NO: 8. In another embodiment, the variant comprises or consists of the substitution L139F as shown in SEQ ID NO: 39.

The amino acid at a position corresponding to position 17 of SEQ ID NO: 7 or corresponding to position 19 of SEQ ID NO: 21 is substituted with Ala, Arg, Asn, Asp, Cys, Gln, Glu, Gly, His, Phe, Leu, Lys, Met, Pro, Ser, Thr, Trp, Tyr, or Val, preferably with Ile. In another embodiment, the variant comprises or consists of the substitution T17I as shown in SEQ ID NO: 18. In another embodiment, the variant comprises or consists of the substitution S19I as shown in SEQ ID NO: 36.

In another embodiment, the alteration of said polypeptide at the position corresponding to position 137 of SEQ ID NO: 7 or corresponding to position 139 of SEQ ID NO: 21 is an amino acid substitution, such as a substitution of leucine by isoleucine, valine, histidine, lysine, methionine, threonine, tryptophan, asparagine, aspartic acid/aspartate, alanine, arginine, cysteine/cystine, glutamine, glutamic acid/glutamate, glycine, proline, serine, or tyrosine.

In another embodiment, the alteration of said polypeptide at the position corresponding to position 17 of SEQ ID NO: 7 is an amino acid substitution, such as a substitution of threonine by isoleucine, valine, histidine, lysine, methionine, leucine, phenylalanine, tryptophan, asparagine, aspartic acid/aspartate, alanine, arginine, cysteine/cystine, glutamine, glutamic acid/glutamate, glycine, proline, serine, or tyrosine.

In another embodiment, the alteration of said polypeptide at the position corresponding to position 19 of SEQ ID NO: 21 is an amino acid substitution, such as a substitution of serine by isoleucine, valine, histidine, lysine, methionine, leucine, phenylalanine, tryptophan, asparagine, aspartic acid/aspartate, alanine, arginine, cysteine/cystine, glutamine, glutamic acid/glutamate, glycine, proline, threonine, or tyrosine.

In one embodiment the alteration of said polypeptide at the position corresponding to position 17 and/or 137 of SEQ ID NO: 7 or corresponding to position 19 and/or 139 of SEQ ID NO: 21 is an amino acid insertion, such as an amino acid insertion of at least one amino acid selected from the list of leucine, phenylalanine, isoleucine, valine, histidine, lysine, methionine, threonine, tryptophan, asparagine, aspartic acid/aspartate, alanine, arginine, cysteine/cystine, glutamine, glutamic acid/glutamate, glycine, proline, serine, or tyrosine. The amino acid insertion can be an insertion of one, two, three, four, five, or more than five amino acids, wherein the amino acids are independently chosen from one another.

In a preferred embodiment of the fifth aspect, the polynucleotide encoding the polypeptide has a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100% to SEQ ID NO: 3, SEQ ID NO: 4, SEQ ID NO: 28, or SEQ ID NO: 40.

In another preferred embodiment, the polynucleotide encoding the polypeptide has a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100% to SEQ ID NO: 3 and comprises at least one nucleotide alteration at a position corresponding to position 495, 496 and/or 497 of SEQ ID NO: 3.

In another preferred embodiment, the polynucleotide encoding the polypeptide has a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100% to SEQ ID NO: 28 and comprises at least one nucleotide alteration at a position corresponding to position 656, 657 and/or 658 of SEQ ID NO: 28.

In one embodiment the at least one nucleotide alteration is a substitution; preferably the nucleotide(s) at a position corresponding to position 495, 496, and/or 497 of SEQ ID NO: 3 or corresponding to position 656, 657 and/or 658 of SEQ ID NO: 28 is/are independently from another substituted with adenine (A), thymine (T), guanine (G) and/or cytosine (C).

In a preferred embodiment, the at least one nucleotide alteration comprises or consists of a substitution of cytosine with thymine at a position corresponding to position 495 of SEQ ID NO: 3, C495T, as shown in SEQ ID NO: 5. Alternatively, the at least one nucleotide alteration comprises or consists of a substitution of cytosine with thymine at a position corresponding to position 495 of SEQ ID NO: 3, C495T, and of a substitution of thymine with cytosine at a position corresponding to position 497 of SEQ ID NO: 3.

In one embodiment, one, two or three of the nucleotides corresponding to position 495, 496 and/or 497 of SEQ ID NO: 3 or corresponding to position 656, 657 and/or 658 of SEQ ID NO: 28 are independently from another substituted with adenine (A), thymine (T), guanine (G) or cytosine (C).

In another embodiment, one, two or three of the nucleotides corresponding to position 495, 496 and/or 497 of SEQ ID NO: 3 or corresponding to position 656, 657 and/or 658 of SEQ ID NO: 28 are, independently from another, deleted.

In another preferred embodiment, the polynucleotide encoding the polypeptide has a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100% to SEQ ID NO: 4 and comprises at least one nucleotide alteration at a position corresponding to position 409, 410 and/or 411 of SEQ ID NO: 4.

In one embodiment the at least one nucleotide alteration is a substitution; preferably the nucleotide(s) at a position corresponding to position 409, 410, and/or 411 of SEQ ID NO: 4 is/are independently from another substituted with adenine (A), thymine (T), guanine (G) and/or cytosine (C).

In a preferred embodiment, the at least one nucleotide alteration comprises or consists of a substitution of cytosine with thymine at a position corresponding to position 409 of SEQ ID NO: 4, C409T, as shown in SEQ ID NO: 6. Alternatively, the at least one nucleotide alteration comprises or consists of a substitution of cytosine with thymine at a position corresponding to position 495 of SEQ ID NO: 4, C409T, and of a substitution of thymine with cytosine at a position corresponding to position 411 of SEQ ID NO: 4.

In one embodiment, one, two or three of the nucleotides corresponding to position 409, 410 and/or 411 of SEQ ID NO: 4 are independently from another substituted with adenine (A), thymine (T), guanine (G) or cytosine (C).

In another embodiment, one, two or three of the nucleotides corresponding to position 409, 410 and/or 411 of SEQ ID NO: 4 are, independently from another, deleted.

In another preferred embodiment, the polynucleotide encoding the polypeptide has a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100% to SEQ ID NO: 3 and comprises at least one nucleotide alteration at a position corresponding to position 49, 50 and/or 51 of SEQ ID NO: 3.

In one embodiment the at least one nucleotide alteration is a substitution; preferably the nucleotide(s) at a position corresponding to position 49, 50, and/or 51 of SEQ ID NO: 3 is/are independently from another substituted with adenine (A), thymine (T), guanine (G) and/or cytosine (C).

In another preferred embodiment, the polynucleotide encoding the polypeptide has a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100% to SEQ ID NO: 28 and comprises at least one nucleotide alteration at a position corresponding to position 55, 56 and/or 57 of SEQ ID NO: 28.

In one embodiment the at least one nucleotide alteration is a substitution; preferably the nucleotide(s) at a position corresponding to position 55, 56, and/or 57 of SEQ ID NO: 28 is/are independently from another substituted with adenine (A), thymine (T), guanine (G) and/or cytosine (C). In a preferred embodiment, the at least one nucleotide alteration comprises or consists of a substitution of cytosine with thymine at a position corresponding to position 50 of SEQ ID NO: 3, C50T, as shown in SEQ ID NO: 16. Alternatively, the at least one nucleotide alteration comprises or consists of a substitution of cytosine by thymine at a position corresponding to position 50 of SEQ ID NO: 3, C50T, and of a substitution of adenine with thymine at a position corresponding to position 51 of SEQ ID NO: 3. Alternatively, the at least one nucleotide alteration comprises or consists of a substitution of cytosine by thymine at a position corresponding to position 50 of SEQ ID NO: 3, C50T, and of a substitution of adenine with cytosine at a position corresponding to position 51 of SEQ ID NO: 3.

In one embodiment, one, two or three of the nucleotides corresponding to position 49, 50 and/or 51 of SEQ ID NO: 3 are independently from another substituted with adenine (A), thymine (T), guanine (G) or cytosine (C).

In another embodiment, one, two or three of the nucleotides corresponding to position 49, 50 and/or 51 of SEQ ID NO: 3 are, independently from another, deleted.

In one embodiment, one, two or three of the nucleotides corresponding to position 55, 56 and/or 57 of SEQ ID NO: 28 are independently from another substituted with adenine (A), thymine (T), guanine (G) or cytosine (C).

In another embodiment, one, two or three of the nucleotides corresponding to position 55, 56 and/or 57 of SEQ ID NO: 28 are, independently from another, deleted.

In another preferred embodiment, the polynucleotide encoding the polypeptide has a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100% to SEQ ID NO: 4 and comprises at least one nucleotide alteration at a position corresponding to position 49, 50 and/or 51 of SEQ ID NO: 4.

In one embodiment the at least one nucleotide alteration is a substitution; preferably the nucleotide(s) at a position corresponding to position 49, 50, and/or 51 of SEQ ID NO: 4 is/are independently from another substituted with adenine (A), thymine (T), guanine (G) and/or cytosine (C).

In a preferred embodiment, the at least one nucleotide alteration comprises or consists of a substitution of cytosine by thymine at a position corresponding to position 50 of SEQ ID NO: 4, C50T, as shown in SEQ ID NO: 17. Alternatively, the at least one nucleotide alteration comprises or consists of a substitution of cytosine by thymine at a position corresponding to position 50 of SEQ ID NO: 4, C50T, and of a substitution of adenine with thymine at a position corresponding to position 51 of SEQ ID NO: 4. Alternatively, the at least one nucleotide alteration comprises or consists of a substitution of cytosine by thymine at a position corresponding to position 50 of SEQ ID NO: 4, C50T, and of a substitution of adenine with cytosine at a position corresponding to position 51 of SEQ ID NO: 4.

In one embodiment, one, two or three of the nucleotides corresponding to position 49, 50 and/or 51 of SEQ ID NO: 4 are independently from another substituted with adenine (A), thymine (T), guanine (G) or cytosine (C).

In another embodiment, one, two or three of the nucleotides corresponding to position 49, 50 and/or 51 of SEQ ID NO: 4 are, independently from another, deleted.

In another preferred embodiment the at least one nucleotide alteration leads to a missense mutation and/or a premature stop codon, wherein the polynucleotide encoding the polypeptide has a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100% to SEQ ID NO: 3 and comprises at least one nucleotide alteration at a position corresponding to position 43, 44 and/or 45 of SEQ ID NO: 3.

In one embodiment the at least one nucleotide alteration is a substitution; preferably the nucleotide(s) at a position corresponding to position 43, 44, and/or 45 of SEQ ID NO: 3 is/are independently from another substituted with adenine (A), thymine (T), guanine (G) and/or cytosine (C) and lead to a premature stop codon at the amino acid position corresponding to position 15 of SEQ ID NO: 7.

In a preferred embodiment, the at least one nucleotide alteration comprises or consists of a substitution of cytosine with thymine at a position corresponding to position 43 of SEQ ID NO: 3, C43T, as shown in SEQ ID NO: 13, leading to a premature stop codon at the amino acid position corresponding to position 15 of SEQ ID NO: 7. Alternatively, the at least one nucleotide alteration comprises or consists of a substitution of cytosine by thymine at a position corresponding to position 43 of SEQ ID NO: 3, C43T, and of a substitution of guanine by adenine at a position corresponding to position 44 of SEQ ID NO: 3, G44A, leading to a premature stop codon at the amino acid position corresponding to position 15 of SEQ ID NO: 7. Alternatively, the at least one nucleotide alteration comprises or consists of a substitution of cytosine by thymine at a position corresponding to position 43 of SEQ ID NO: 3, C43T, and of a substitution of guanine by adenine at a position corresponding to position 44 of SEQ ID NO: 3, G44A, and of a substitution of adenine by guanine at a position corresponding to position 45 of SEQ ID NO: 3, A45G leading to a premature stop codon at the amino acid position corresponding to position 15 of SEQ ID NO: 7.

In one embodiment, one, two or three of the nucleotides corresponding to position 43, 44 and/or 45 of SEQ ID NO: 3 are independently from another substituted with adenine (A), thymine (T), guanine (G) or cytosine (C), leading to a premature stop codon at the amino acid position corresponding to position 15 of SEQ ID NO: 7.

In another embodiment, one, two or three of the nucleotides corresponding to position 43, 44 and/or 45 of SEQ ID NO: 3 are, independently from another deleted, leading to a premature stop codon at the amino acid position corresponding to position 15 of SEQ ID NO: 7, or to a premature stop codon at an amino acid position corresponding to a position beyond position 15 or SEQ ID NO: 7.

In another preferred embodiment, the polynucleotide encoding the polypeptide has a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100% to SEQ ID NO: 4 and comprises at least one nucleotide alteration at a position corresponding to position 43, 44 and/or 45 of SEQ ID NO: 4, leading to a premature stop codon at the amino acid position corresponding to position 15 of SEQ ID NO: 7 or downstream thereof.

In one embodiment the at least one nucleotide alteration is a substitution; preferably the nucleotide(s) at a position corresponding to position 43, 44, and/or 45 of SEQ ID NO: 4 is/are independently from another substituted with adenine (A), thymine (T), guanine (G) and/or cytosine (C), leading to a premature stop codon at the amino acid position corresponding to position 15 of SEQ ID NO: 7.

In a preferred embodiment, the at least one nucleotide alteration comprises or consists of a substitution of cytosine with thymine at a position corresponding to position 43 of SEQ ID NO: 4, C43T, as shown in SEQ ID NO: 14, leading to a premature stop codon at the amino acid position corresponding to position 15 of SEQ ID NO: 7. Alternatively, the at least one nucleotide alteration comprises or consists of a substitution of cytosine by thymine at a position corresponding to position 43 of SEQ ID NO: 4, C43T, and of a substitution of guanine by adenine at a position corresponding to position 44 of SEQ ID NO: 4, G44A, leading to a premature stop codon at the amino acid position corresponding to position 15 of SEQ ID NO: 7 or downstream thereof. Alternatively, the at least one nucleotide alteration comprises or consists of a substitution of cytosine by thymine at a position corresponding to position 43 of SEQ ID NO: 4, C43T, and of a substitution of guanine by adenine at a position corresponding to position 44 of SEQ ID NO: 4, G44A, and of a substitution of adenine by guanine at a position corresponding to position 45 of SEQ ID NO: 4, A45G leading to a premature stop codon at the amino acid position corresponding to position 15 of SEQ ID NO: 7 or downstream thereof.

In one embodiment, one, two or three of the nucleotides corresponding to position 43, 44 and/or 45 of SEQ ID NO: 4 are independently from another substituted with adenine (A), thymine (T), guanine (G) or cytosine (C), leading to a premature stop codon at the amino acid position corresponding to position 15 of SEQ ID NO: 7 or downstream thereof.

In another embodiment, one, two or three of the nucleotides corresponding to position 43, 43 and/or 45 of SEQ ID NO: 4 are, independently from another deleted, leading to a premature stop codon at the amino acid position corresponding to position 15 of SEQ ID NO: 7 or beyond position 15.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

Altering, Reduction, or Elimination of Man₅GlcNAc₂-PP-Dol alpha-1,3-mannosyltransferase

### Activity

The present invention also relates to methods of producing a mutant of a parent cell, which comprises disrupting or deleting a polynucleotide, or a portion thereof, encoding a polypeptide of the present invention, which results in the mutant cell producing less or none of the functional polypeptide than the parent cell when cultivated under the same conditions.

The mutant cell may be constructed by reducing or eliminating expression of the polynucleotide using methods well known in the art, for example, insertions, disruptions, replacements, or deletions. In some embodiments, the polynucleotide is reduced or inactivated. The polynucleotide to be modified, reduced or inactivated may be, for example, the coding region or a part thereof essential for activity, or a regulatory element required for expression of the coding region. An example of such a regulatory or control sequence may be a promoter sequence or a functional part thereof, i.e., a part that is sufficient for affecting expression of the polynucleotide. Other control sequences for possible modification include, but are not limited to, a leader, polyadenylation sequence, propeptide sequence, signal peptide sequence, transcription terminator, and transcriptional activator.

Modification, reduction or inactivation of the polynucleotide may be performed by subjecting the parent cell to mutagenesis and selecting for mutant cells in which expression of the polynucleotide has been reduced or eliminated. The mutagenesis, which may be specific or random, may be performed, for example, by use of a suitable physical or chemical mutagenizing agent, by use of a suitable oligonucleotide, or by subjecting the DNA sequence to PCR generated mutagenesis. Furthermore, the mutagenesis may be performed by use of any combination of these mutagenizing agents.

Examples of a physical or chemical mutagenizing agent suitable for the present purpose include ultraviolet (UV) irradiation, hydroxylamine, N-methyl-N′-nitro-N-nitrosoguanidine (MNNG), O-methyl hydroxylamine, nitrous acid, ethyl methane sulphonate (EMS), sodium bisulphite, formic acid, and nucleotide analogues.

When such agents are used, the mutagenesis is typically performed by incubating the parent cell to be mutagenized in the presence of the mutagenizing agent of choice under suitable conditions, and screening and/or selecting for mutant cells exhibiting reduced or no expression of the gene.

Modification, reduction or inactivation of the polynucleotide may be accomplished by insertion, substitution, or deletion of one or more nucleotides in the gene or a regulatory element required for transcription or translation thereof. For example, nucleotides may be inserted or removed so as to result in the introduction of a premature stop codon, the removal of the start codon, or a change in the open reading frame. Such modification or inactivation may be accomplished by site-directed mutagenesis or PCR generated mutagenesis in accordance with methods known in the art. Although, in principle, the modification may be performed in vivo, i.e., directly on the cell expressing the polynucleotide to be modified, it is preferred that the modification be performed in vitro as exemplified below.

An example of a convenient way to alter, eliminate or reduce expression of a polynucleotide is based on techniques of gene replacement, gene deletion, or gene disruption. For example, in the gene disruption method, a nucleic acid sequence corresponding to the endogenous polynucleotide is mutagenized in vitro to produce a defective nucleic acid sequence that is then transformed into the parent cell to produce a defective gene. By homologous recombination, the defective nucleic acid sequence replaces the endogenous polynucleotide. It may be desirable that the defective polynucleotide also encodes a marker that may be used for selection of transformants in which the polynucleotide has been modified or destroyed. In an aspect, the polynucleotide is disrupted with a selectable marker such as those described herein.

The present invention further relates to a mutant cell of a parent cell that comprises a disruption or deletion of a polynucleotide encoding the polypeptide or a control sequence thereof or a silenced gene encoding the polypeptide, which results in the mutant cell producing less of the functional polypeptide, no polypeptide compared to the parent cell, or no functional polypeptide compared to the parent cell.

The polypeptide-deficient mutant cells are useful as host cells for expression of native and heterologous polypeptides. Therefore, the present invention further relates to methods of producing a native or heterologous polypeptide, comprising (a) cultivating the mutant cell under conditions conducive for production of the polypeptide; and (b) recovering the polypeptide. The term “heterologous polypeptides” means polypeptides that are not native to the host cell, e.g., a variant of a native protein. The host cell may comprise more than one copy of a polynucleotide encoding the native or heterologous polypeptide.

The methods used for cultivation and purification of the product of interest may be performed by methods known in the art.

The methods of the present invention for producing in host cells which are essentially free of or have reduced activity of Man₅GlcNAc₂-PP-Dol alpha-1,3-mannosyltransferase are of interest in the production of polypeptides, e.g., fungal proteins such as enzymes. The Man₅GlcNAc₂-PP-Dol alpha-1,3-mannosyltransferase-reduced/-deficient cells may also be used to express heterologous proteins of pharmaceutical interest such as hormones, growth factors, receptors, and the like.

The present invention is further described by the following examples that should not be construed as limiting the scope of the invention.

### EXAMPLES

### Materials and Methods

Unless otherwise stated, DNA manipulations and transformations were performed using standard methods of molecular biology as described in Sambrook et al. (1989) Molecular cloning: A laboratory manual, Cold Spring Harbor lab., Cold Spring Harbor, NY; Ausubel, F. M. et al. (eds.) “Current protocols in Molecular Biology”, John Wiley and Sons, 1995; Harwood, C. R., and Cutting, S. M. (eds.) “Molecular Biological Methods for Bacillus”. John Wiley and Sons, 1990.

Purchased Material (E. coli and Kits)

E. coli DH5α (Toyobo) is used for plasmid construction and amplification. Amplified plasmids are recovered with Qiagen Plasmid Kit (Qiagen). Ligation is done with either Rapid DNA Dephos & Ligation Kit (Roche) or In-Fusion kit (Clontech Laboratories, Inc.) according to the manufactory instructions. Polymerase Chain Reaction (PCR) is carried out with KOD-Plus system (TOYOBO). Fungal spore-PCR was conducted by using Phire® Plant Direct PCR Kit (New England Biolabs). QIAquick™ Gel Extraction Kit (Qiagen) is used for the purification of PCR fragments and extraction of DNA fragment from agarose gel.

### Enzymes

Enzymes for DNA manipulations (e.g. restriction endonucleases, ligases etc.) are obtainable from New England Biolabs, Inc. and were used according to the manufacturer's instructions.

### Plasmids

pBluescript II SK- (Stratagene #212206)

The pHUda801 harbouring A. nidulans pyrG gene and herpes simplex virus (HSV) thymidine kinase gene (tk) driven by A. nidulans glyceraldehyde-3-phosphate dehydrogenase promoter (Pgpd), A. nidulans tryptophane synthase terminator (TtrpC) and A. nigerglucoamylase terminator (Tamg) are described in Example 4 and Example 5 in WO2012/160093.

The amino acid sequence for Gs AMG harboring the amyloglucosidase from Gloeophyllum sepiarium is identified as SEQ ID NO: 11. The amino acid sequence for PE variant of glucoamylase from Gloeophyllum sepiarium is identified as SEQ ID NO: 9.

### Microbial Strains

The expression host strain Aspergillus niger C5644 was isolated by the Applicant and is a derivative of Aspergillus niger NNO49184 which was isolated from soil as described in example 14 in WO2012/160093. C5644 is a strain which produces the glucoamylase with SEQ ID NO: 9.

Trichoderma reesei BTR213 has been described in WO 2013/086633.

Trichoderma reesei strain 6Q-M1002 is a ku70 disrupted and paracelsin synthetase (parS) deleted strain derived from T. reesei BTR213. The cellobiohydrolase I (cbh1), cellobiohydrolase II (cbh2), endoglucanase I (eg1), endoglucanase II (eg2), and endoglucanase III (eg3) genes are deleted in this strain. There is a ˜20 kb deletion between the cbh2 and the eg2 loci caused by the FRT-F/FRT-F3 recombination. In addition, TF92949 (disclosed in WO2020/123845) has been deleted in the strain as well. Four copies of Acremonium alcalophilum CBS114.92 lysozyme (SEQ ID NO: 20) expression cassette (driven by the cbh1 promoter) flanked by FRT-F and FRT-F3 sites have been integrated into the strain.

### Medium

COVE trace metals solution was composed of 0.04 g of NaB4O7·10H2O, 0.4 g of CuSO4·5H2O, 1.2 g of FeSO4·7H2O, 0.7 g of MnSO4·H2O, 0.8 g of Na2MoO2·2H2O, 10 g of ZnSO4·7H2O, and deionized water to 1 liter.

50× COVE salts solution was composed of 26 g of KCl, 26 g of MgSO4·7H2O, 76 g of KH2PO4, 50 ml of COVE trace metals solution, and deionized water to 1 liter.

COVE medium was composed of 342.3 g of sucrose, 20 ml of 50× COVE salts solution, 10 ml of 1 M acetamide, 10 ml of 1.5 M CsCl2, 25 g of Noble agar, and deionized water to 1 liter.

COVE-N-Gly plates were composed of 218 g of sorbitol, 10 g of glycerol, 2.02 g of KNO3, 50 ml of COVE salts solution, 25 g of Noble agar, and deionized water to 1 liter.

COVE-N (tf) was composed of 342.3 g of sucrose, 3 g of NaNO3, 20 ml of COVE salts solution, 30 g of Noble agar, and deionized water to 1 liter.

COVE-N top agarose was composed of 342.3 g of sucrose, 3 g of NaNO3, 20 ml of COVE salts solution, 10 g of low melt agarose, and deionized water to 1 liter.

COVE-N was composed of 30 g of sucrose, 3 g of NaNO3, 20 ml of COVE salts solution, 30 g of Noble agar, and deionized water to 1 liter.

STC buffer was composed of 0.8 M sorbitol, 25 mM Tris pH 8, and 25 mM CaCl2.

STPC buffer was composed of 40% PEG 4000 in STC buffer.

LB medium was composed of 10 g of tryptone, 5 g of yeast extract, 5 g of sodium chloride, and deionized water to 1 liter.

LB plus ampicillin plates were composed of 10 g of tryptone, 5 g of yeast extract, 5 g of sodium chloride, 15 g of Bacto agar, ampicillin at 100 μg per ml, and deionized water to 1 liter.

YPG medium was composed of 10 g of yeast extract, 20 g of Bacto peptone, 20 g of glucose, and deionized water to 1 liter.

SOC medium was composed of 20 g of tryptone, 5 g of yeast extract, 0.5 g of NaCl, 10 ml of 250 mM KCl, and deionized water to 1 liter.

TAE buffer was composed of 4.84 g of Tris Base, 1.14 ml of Glacial acetic acid, 2 ml of 0.5 M EDTA pH 8.0, and deionized water to 1 liter.
MSS is composed of 72 g Glycerol, 92 g Soybean powder (pH 6.0), water to 1 litre.
MU-1 is composed 260 g of Maltodextrin, 3 g of MgSO₄·7H₂O, 5 g of KH₂PO₄, 6 g of K₂SO₄, amyloglucosidase trace metal solution 0.5 ml and urea 2 g (pH 4.5), water to 1 liter.

Fermentation batch medium for T. reesei was composed of 24 g of dextrose, 40 g of soy meal, 8 g of (NH₄)₂SO₄, 3 g of K₂HPO₄, 8 g of K₂SO₄, 3 g of CaCO₃, 8 g of MgSO₄·7H₂O, 1 g of citric acid, 8.8 ml of 85% phosphoric acid, 1 ml of anti-foam, 14.7 ml of trace metals solution, and deionized water to 1 liter.

Trace metals solution for T. reesei fermentation was composed of 26.1 g of FeSO₄·7H₂O, 5.5 g of ZnSO₄·7H₂O, 6.6 g of MnSO₄·H₂O, 2.6 g of CuSO₄·5H₂O, 2 g of citric acid, and deionized water to 1 liter. The solution was sterilized by autoclaving.

Fermentation feed medium for T. reesei was composed of 1190 g glucose, 14.2 ml 85% H3PO4 and 486 g H₂O. The solution was sterilized by autoclaving.

Sample buffer (pH 7.5) was composed of 0.1 M Tris-HCl, 0.1 M NaCl and 0.01% Triton X-100. The solution was filter sterilized.

Shake flask medium was composed of 20 g of glycerol, 10 g of soy meal, 1.5 g of (NH₄)₂SO₄, 2 g of KH₂PO₄, 0.2 g of CaCl₂, 0.4 g of MgSO₄·7H₂O, 0.2 ml of trace metals solution, and deionized water to 1 liter.

COVE2 plates were composed of 30 g of sucrose, 20 ml of COVE salts solution, 10 ml of 1 M acetamide, 25 g of Difco™ agar Noble, and deionized water to 1 liter. The solution was sterilized by autoclaving.

PDA+1 M sucrose plates were composed of 39 g of Difco™ potato dextrose agar, 342.30 g sucrose and deionized water to 1 liter. The solution was sterilized by autoclaving.

PEG buffer was composed of 50% polyethylene glycol (PEG) 4000, 10 mM Tris-HCl pH 7.5, and 10 mM CaCl₂ in deionized water. The solution was filter sterilized.

Sample buffer (pH 7.5) was composed of 0.1 M Tris-HCl, 0.1 M NaCl and 0.01% Triton X-100. The solution was filter sterilized. Shake flask medium was composed of 20 g of glycerol, 10 g of soy meal, 1.5 g of (NH₄)₂SO₄, 2 g of KH₂PO₄, 0.2 g of CaCl₂, 0.4 g of MgSO₄·7H₂O, 0.2 ml of trace metals solution, and deionized water to 1 liter.

1.2 M sorbitol was composed of 218.4 g sorbitol and deionized water to 1 liter. The solution was sterilized by autoclaving.

Tr-STC was composed of 1 M sorbitol, 10 mM Tris-HCl pH 7.5, and 50 mM CaCl₂ in deionized water. The solution was filter sterilized.

TBE buffer was composed of 10.8 g of Tris Base, 5 g of boric acid, 4 ml of 0.5 M EDTA pH 8, and deionized water to 1 liter.

TE buffer is composed of 1 M Tris-HCl pH 8.0 and 0.5 M EDTA pH 8.0.

Transformation of Aspergillus niger

Transformation of Aspergillus species can be achieved using the general methods for yeast transformation. The preferred procedure for the invention is described below.

Aspergillus niger host strain was inoculated to 100 ml of YPG medium supplemented with 10 mM uridine and incubated for 16 hrs at 32° C. at 80 rpm. Pellets were collected and washed with 0.6 M KCl, and resuspended 20 ml 0.6 M KCl containing a commercial β-glucanase product (GLUCANEX™, Novozymes A/S, Bagsværd, Denmark) at a final concentration of 20 mg per ml. The suspension was incubated at 32° C. at 80 rpm until protoplasts were formed, and then washed twice with STC buffer. The protoplasts were counted with a hematometer and resuspended and adjusted in an 8:2:0.1 solution of STC:STPC:DMSO to a final concentration of 2.5×10⁷ protoplasts/ml. Approximately 4 μg of plasmid DNA was added to 100 μl of the protoplast suspension, mixed gently, and incubated on ice for 30 minutes. One ml of SPTC was added and the protoplast suspension was incubated for 20 minutes at 37° C. After the addition of 10 ml of 50° C. Cove or Cove-N top agarose, the reaction was poured onto Cove or Cove-N (tf) agar plates and the plates were incubated at 32° C. for 5 days.

PCR amplifications in Examples

Polymerase Chain Reaction (PCR) was carried out with KOD-Plus system (TOYOBO).

| Component | Volume | | Final Concentration | |
|---|---|---|---|---|
| 10x Buffer for KOD -Plus- | 5 | μl | 1x | |
| 2 mM dNTPs | 5 | μl | 0.2 mM each | |
| 25 mM MgSO₄ | 2 | μl | 1.0 | mM |
| 10 pmol/μl Primer #1 | 1.5 | μl | 0.3 | μM |
| 10 pmol/μl Primer #2 | 1.5 | μl | 0.3 | μM |

| Template DNA | X | μl | |
|---|---|---|---|
| Genomic DNA Plasmid DNA | 10-200 ng/50 μl | | |
| | 1-50 ng/50 μl | | |
| PCR grade water | Y | μl | |
| KOD-Plus- (1.0 U/μl) | 1 | μl | 1.0 U/50 μl |
| Total reaction volume | 50 | μl | |

3-step Cycle:

| | | |
|---|---|---|
| Pre-denaturation: 94° C., 2 min. | | |
| Denaturation: 94° C., 15 sec. | | |
| Annealing: Tm-[5-10] ° C.*, 30 sec. | {close oversize brace} | 35 cycles |
| Extension: 68° C., 1 min./kb | | |

### Southern Hybridization

Each of the spore purified transformants were cultivated in 3 ml of YPG medium and incubated at 30° C. for 2 days with shaking at 200 rpm. Biomass was collected using a MIRACLOTH® lined funnel. Ground mycelia were subject to genome DNA preparation using FastDNA SPIN Kit for Soil (MP Biomedicals) follows by manufacture's instruction. Non-radioactive probes were synthesized using a PCR DIG probe synthesis kit (Roche Applied Science, Indianapolis IN) followed by manufacture's instruction. DIG labeled probes were gel purified using a QIAquick™ Gel Extraction Kit (QIAGEN Inc., Valencia, CA) according to the manufacturer's instructions.

Five micrograms of genome DNA was digested with appropriate restriction enzymes completely for 16 hours (40 μl total volume, 4 U enzyme/μl DNA) and run on a 0.8% agarose gel. The DNA was fragmented in the gel by treating with 0.2 M HCl, denatured (0.5 M NaOH, 1.5 M NaCl) and neutralized (1 M Tris, pH7.5; 1.5 M NaCl) for subsequent transfer in 20×SSC to Hybond N+ membrane (Amersham). The DNA was UV cross-linked to the membrane and prehybridized for 1 hour at 42° C. in 20 ml DIG Easy Hyb (Roche Diagnostics Corporation, Mannheim, Germany). The denatured probe was added directly to the DIG Easy Hyb buffer and an overnight hybridization at 42° C. was done. Following the post hybridization washes (twice in 2×SSC, room temperature, 5 min and twice in 0.1×SSC, 68° C., 15 min. each), chemiluminescent detection using the DIG detection system and CPD-Star (Roche) was done followed by manufacture's protocol. The DIG-labeled DNA Molecular Weight Marker II (Roche) was used for the standard marker.

### Shaking Flask Cultivation for Glucoamylase Production

Spores of the selected transformants were inoculated in 100 ml of MSG media and cultivated at 30° C. for 3 days with shaking (220 rpm). 10% of seed culture was transferred to MU-1 medium and cultivated at 32° C. for 7 days with shaking (220 rpm). The supernatant was obtained by centrifugation and used for sub-sequent assay.

### Glucoamylase Activity

Glucoamylase activity was determined by RAG assay method (Relative AG assay, pNPG method). pNPG substrate was composed of 0.1 g of p-Nitrophenyl-α-D-glycopyranoside (Nacalai Tesque), 10 ml of 1 M Acetate buffer (pH 4.3) and deionized water to 100 ml. From each diluted sample solution, 40 ul is added to well in duplicates for “Sample”. And 40 ul deionized water is added to a well for “Blank”. And 40 ul of AG standard solution is added as “Reference”. Using Multidrop (Labsystem), 80 ul of pNPG substrate is added to each well. After 20 minutes at room temperature, the reaction is stopped by addition of 120 ul of Stop reagent (0.1M Borax solution). OD values are measured by microplate reader at 400 nm (Power Wave X) or at 405 nm (ELx808).

Calculation was conducted as follows:

$$\frac{( S - B ) \times F \times AGs}{( Ss - Bs )} = RAG / ml$$

$$\begin{matrix}S = Sample value & F = dilution factor \\ B = Blank value & AGs = AG / ml of the AG standard \\ Ss = Value of AG standard & \\ Bs = Blank of AG standard & \end{matrix} .$$

### Purification of the GSA202 Proteins:

First, alpha-cyclodextrin affinity column was prepared as follows. Ten grams of Epoxy-activated Sepharose 6B (GE Healthcare, Chalfont St. Giles, U. K) powder was suspended in and washed with distilled water on a sintered glass filter. The gel was suspended in coupling solution (100 ml of 12.5 mg/ml alpha-cyclodextrin, 0.5 M NaOH) and incubated at room temperature for one day with gentle shaking. The gel was washed with distilled water on a sintered glass filter, suspended in 100 ml of 1 M ethanolamine, pH 10, and incubated at 50° C. for 4 hours for blocking. The gel was then washed several times using 50 mM Tris-HCl, pH 8 and 50 mM NaOAc, pH 4.0 alternatively. The gel was finally packed into column using equilibration buffer (50 mM NaOAc, 150 mM NaCl, pH 4.5).

Next, purification of GSA202 proteins was done as follows. The supernatant of fermentation sample was filtered using a filtration unit equipped with a 0.22 μm filter (Millipore). The filtered supernatant was applied to a 15 ml Alpha-cyclodextrin affinity column (pre-equilibrated with 5 column volumes (CV) of Buffer 1 (20 mM NaOAc pH5, 1 mM CaCl2). Unbound protein was eluted by washing the column with 3 CV of Buffer 1. The target enzyme was eluted with 20 mM NaOAc pH5, 10 mM beta-cyclodextrin, 1 mM CaCl2 at a flow rate of 5 ml/minute and elution was monitored by absorbance at 280 nm. The eluted enzyme was applied to a HiLoad™ 26/60 Superdex 200 prep grad column (GE Healthcare Life Sciences) pre-equilibrated with 3 CV of Buffer 1. The enzyme was eluted from the column using Buffer 1 at a flow rate of 2.6 ml/minute. Relevant fractions were selected and pooled based on the chromatogram and SDS-PAGE analysis using 12% Mini-PROTEAN TGX Stain-free gels (BIO-RAD). The concentration of the purified enzyme was determined by absorbance at 280 nm.

### Determination of Molecular Weight:

The intact molecular weight analyses were performed using a MAXIS II electrospray mass spectrometer (Bruker Daltonik GmbH, Bremen, DE). The samples were first diluted to 0.2 mg/ml in 50 mM NH4Ac pH5.5. The diluted samples were applied to an AdvanceBio Desalting-RP column (Agilent Technologies) followed by washing and elution from the column running an acetonitrile linear gradient and introduced to the electrospray source with a flow of 400 ml/min by an Ultimate 3000 LC system (Dionex). Data analysis was performed with DataAnalysis version 4.3 (Bruker Daltonik GmbH, Bremen, DE). The molecular weight of the samples was calculated by deconvolution of the raw data in the range 30.000 to 80.000 Da.

### Specific Activity of Purified GSA202:

The Glucoamylase Unit (AGU) is defined as the amount of enzyme, which hydrolyses 1 micromole maltose per minute under the standardized conditions, thereby generating glucose. The analysis principle is described by 3 reaction steps: Step 1 is an enzyme reaction: Amyloglucosidase (AMG) and exo-alpha-1,4-glucan-glucohydrolase, hydrolyzes maltose to form alpha-D-glucose. After incubation, the reaction is stopped with NaOH. Steps 2 and 3 result in an endpoint reaction: Glucose is phosphorylated by ATP, in a reaction catalyzed by hexokinase. The glucose-6-phosphate formed is oxidized to 6-phosphogluconate by glucose-6-phosphate dehydrogenase. In this same reaction an equimolar amount of NAD+ is reduced to NADH with increase in absorbance at 340 nm. An autoanalyzer system such as Konelab 30 Analyzer (Thermo Fisher Scientific) may be used with following reaction conditions (Table 1).

TABLE 1
Reaction conditions for specific activity measurement

| Parameter | Reaction conditions | |
|---|---|---|
| Temperature [° C.] | 37.0 | |
| pH | Approx. 7.8 | |
| Hexokinase | >0.66 | U/ml |
| Glucose-6-P-DH | >0.66 | U/ml |
| Reaction time [sec] | 420 | |
| Wavelength [nm] | 340 | |
| Triton-X | 4 | mL/L |
| Na⁺ | >40 | mM |
| NAD⁺ | 0.74 | mM |
| ATP | 0.74 | mM |
| Mg²⁺ | 1.41 | mM |
| Tris | 35.2 | mM |

### Example 1 Construction of Plasmids for Integration of Mutations in Alg3 Gene

The purpose of this experiment is to prepare the plasmid for integration of single-nucleotide mutation into native alg3 gene to cause amino acid changes (Leu137Phe) in A. niger strains.

Construction of Single RNA-Guided DNA Endonuclease Plasmids plhar531

One protospacer was designed to target the alg3 gene as seen in Table 2.

TABLE 2

| Plasmid | Protospacer (PS) |
|---|---|
| plhar531 | gccgtacttgcttccgctgc (SEQ ID NO: 1) |

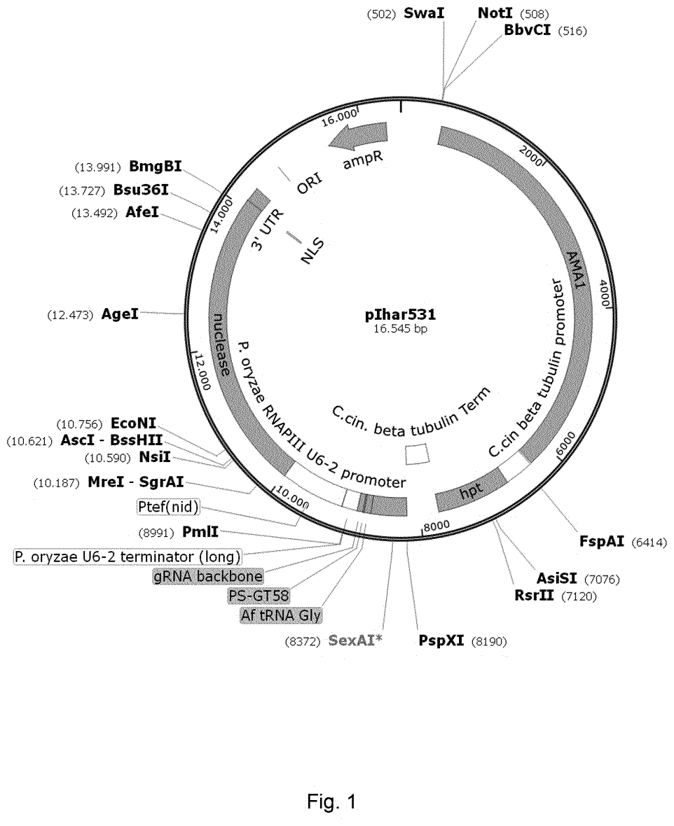

The oligo DNA was described in Table 3. The oligo DNA was inserted into pSMai290 digested by BglII. pSMai290 was digested by BglII and purified by 0.8% agarose gel electrophoresis using TAE buffer, where a 16,531 bp fragment was excised from the gel and extracted using a QIAQUICK® Gel Extraction Kit. This fragment was ligated to the 60-mer oligo DNA by using the In-Fusion kit (Clontech Laboratories, Inc.) according to the manufactory instructions. The reaction was performed at 50° C. for 15 minutes. One μl of the reaction mixture were transformed into DH5α chemically competent E. coli cells. Transformants were spread onto LB plus ampicillin plates and incubated at 37° C. overnight. Plasmid DNA was purified from several transformants using a QIA mini-prep kit. The plasmid DNA was screened for proper ligation by use of proper restriction enzymes followed by 0.8% agarose gel electrophoresis using TAE buffer. The plasmid was designated as plhar531 (FIG. 1).

TABLE 3
The primer for the construction of plhar531

| Plasmid | Oligo DNA |
|---|---|
| plhar531 | IH160M-GT58: ttcgattcacggatgatgcagccg |
| | tacttgcttccgctgcgttttagagctagaaatagc |
| | (SEQ ID NO: 2) |

### Example 2 Transformation of A. niger Strain C5644 with Plhar531 and IH164M for the Integration of the Mutations in Alg3 Gene and Effect of the Integration of Mutations in Alg3 Gene on the Enzyme Production

The purpose of this experiment is to generate transformants expressing alg3 gene with a single-nucleotide substitution in place of the wild-type alg3 gene. plhar531 and IH164M are designed to integrate single-nucleotide substitutions for one amino acid change (Leu137Phe) into alg3 gene.

Sequence analysis was performed to confirm the integration of the mutations by plhar531 and IH164M in alg3 gene after transformation.

Three transformants (531-C5644-4, 531-C5644-8, 531-C5644-10) from C5644 were fermented in 5 liter fermentators and their enzyme activities (amyloglucosidase unit activities=AGU activities) of culture broth were measured followed by the materials and methods described above; results are shown in Table 4. These three transformants (531-C5644-4, 531-C5644-8, and 531-C5644-10) expressing mutated alg3 gene (Leu137Phe) showed in average around 8.9% higher AGU activity than the reference strain C5644 with wild-type alg3 gene, showing that the mutations in alg3 gene can increase enzyme productivity and/or yield of a fungal host strain. The glucoamylase polypeptide of SEQ ID NO: 9 expressed in mutants and control strain showed AGU activity as depicted in Table 4.

TABLE 4

| Strain | AGU relative activity |
|---|---|
| 531-C5644-4 | 1.075 |
| 531-C5644-8 | 1.085 |
| 531-C5644-10 | 1.107 |
| C5644 | 1.00 |

The average AGU activity of the selected three strains from each host strain, wherein the average glucoamylase yields from C5644 is normalized to 1.00.

### Example 3 Introduction of Another Type of a Single Nucleotide Substitution to Alg3 Gene in the A. niger Strain C5644 and its Effect on the Enzyme Production and Activity

The purpose of this experiment is to generate transformants expressing alg3 gene with a single-nucleotide substitution other than Leu137Phe. As described in Example 2, genome editing tools were used to integrate a single nucleotide substitution for a single amino acid change (Arg15*(stop) or Thr17Ile) into alg3 gene. Protospacer (SEQ ID NO: 19) is identical for both mutations Arg15* and Thr17Ile. The introduction of such substitutions was confirmed by sanger sequencing after transformation.

C5644-469-17 (Thr17Ile) and C5644-469-34 (Arg15*) were generated from C5644. These two and reference strains were fermented in shake flasks and AGU activities of culture supernatants were measured as described in materials and methods. Results are shown in Table 5. C5644-469-17 (Thr17Ile) and C5644-469-34 (Arg15*) showed 7.7% and 6.1% higher AGU activity than the reference strain C5644 with wild-type alg3 gene, respectively. This showed that protein yield and/or specific activity of the glucoamylase increased by those amino acid changes introduced in the alg3 gene.

TABLE 5

| | AGU relative | Amino acid | Alg 3 polypeptide |
|---|---|---|---|
| Strain | activity | change in alg3 | SEQ ID NO: |
| C5644-469-17 | 1.077 | Thr17Ile | 18 |
| C5644-469-34 | 1.061 | Arg15* | 15 |
| 531-C5644-8 | 1.086 | Leu137Phe | 8 |
| C5644 | 1.00 | — | 7 |

The average AGU activity of the tested strains, wherein the average glucoamylase yields from 05644 is normalized to 1.00.

TABLE 6
Sequence ID overview

| SEQ ID NO: | | SNP |
|---|---|---|
| 1 | plhar531 protospacer | — |
| 2 | plhar531 primer IH160M-GT58 | — |
| 3 | wild type alg3 gDNA A. niger | None |
| 4 | wild type alg3 cDNA A. niger | None |
| 5 | variant Alg3 gDNA A. niger | L137F |
| 6 | variant Alg3 cDNA A. niger | L137F |
| 7 | wild type alg3 polypeptide A. niger | None |
| 8 | variant alg3 polypeptide A. niger | L137F |
| 9 | variant glucoamylase polypeptide | — |
| 10 | variant glucoamylase nucleotide | — |
| 11 | glucoamylase polypeptide | — |
| 12 | glucoamylase nucleotide | — |
| 13 | Variant alg3 gDNA A. niger | R15* stop |
| 14 | Variant alg3 cDNA A. niger | R15* stop |
| 15 | Variant alg3 polypeptide A. niger | R15* stop |
| 16 | Variant alg3 gDNA A. niger | T17I |
| 17 | Variant alg3 cDNA A. niger | T17I |
| 18 | Variant alg3 polypeptide A. niger | T17I |
| 19 | pTNA469 protospacer | — |
| 20 | CBS114.92 lysozyme nucleotide | — |
| 21 | wild type alg3 polypeptide T. reesei | none |
| 22 | 1238937 oligo | — |
| 23 | 1238938 oligo | — |
| 24 | pAgJg341 protospacer | — |
| 25 | pAgJg342 protospacer | — |
| 26 | 1238939 oligo | — |
| 27 | 1238940 oligo | — |
| 28 | wild type alg3 gDNA T. reesei | none |
| 29 | 1238985 primer | — |
| 30 | 1238986 primer | — |
| 31 | 1238941 primer | — |
| 32 | 1238942 primer | — |
| 33 | CBS114.92 lysozyme polypeptide | — |
| 34 | Variant alg3 gDNA T. reesei | S19I |
| 35 | Variant alg3 cDNA T. reesei | S19I |
| 36 | Variant alg3 polypeptide T. reesei | S19I |
| 37 | Variant alg3 gDNA T. reesei | L139F |
| 38 | Variant alg3 cDNA T. reesei | L139F |
| 39 | Variant alg3 polypeptide T. reesei | L139F |
| 40 | Wildtype alg3 cDNA T. reesei | None |

### Example 4 Characterization of the Protein Products Expressed by Alg3 Mutants

The purpose of this experiment is the evaluation of the protein products expressed by alg3 wildtype strains, the alg3 mutants L137F and T17I, and the alg3 deletion strain R15*. Since the alg3 gene is involved in N-glycosylation of proteins, the glucoamylase product (GSA202) obtained from alg3 mutant strains L137F or T17I might have altered glycosylation when compared to the GSA202 glycosylation of the wildtype alg3 strain or the alg3 deletion strain R15*. To analyze the glycosylation pattern of GSA202 from the alg3 mutant strains, the products were purified from tank-fermented broth and subjected to MS analysis. In addition, specific activity of each product was investigated.

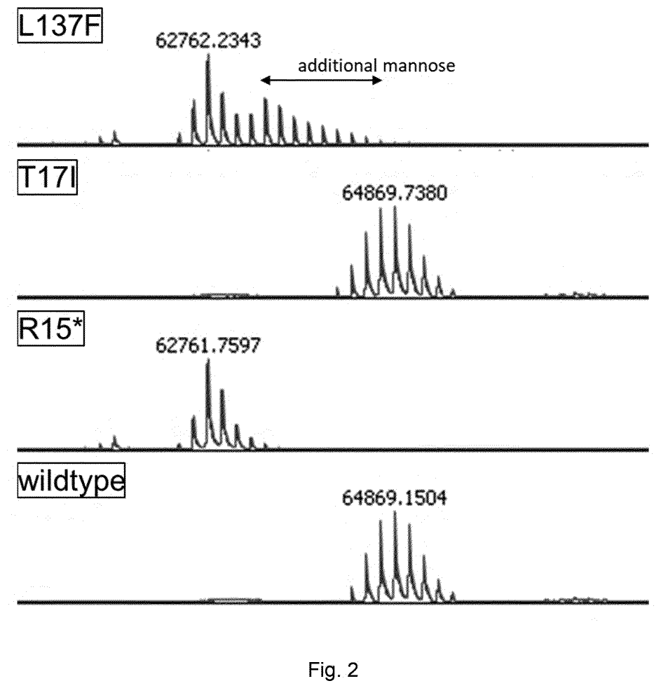

As shown in FIG. 2, MS analyses revealed that the GSA202 products were differently glycosylated, depending on alg3 genotypes. The major peaks and sub-peaks of GSA202 from alg3 wildtype and T17I mutant were very consistent, leading to the assumption that the T17I substitution does not affect the alg3 function by means of product glycosylation. However, since T17 is located in N-terminal position of the ALG3 protein, the T17I substitution may have impact on other roles, such as signal recognition. Said impact lead to increased GSA202 yield/relative activity of ca. 7.7%, whilst alg3 deletion mutant R15* achieved only an increased product yield/relative activity of ca. 6.1% (see Table 5 of Example 3). An additional disadvantage of the R15* mutant is that compared to wildtype alg3 the R15* mutant produced GSA202 with altered glycosylation patterns as can be seen in FIG. 2. Therefore, it is suggested that alg3 modulation, such as the T17I substitution, has a more positive impact on protein productivity and protein quality than alg3 deletion represented by the R15* mutant, without compromising the increase in relative product activity (Table 5) or specific product activity (Table 7) or cell growth, which has been discussed to be negatively impacted by alg3 deletion by Dai Z. et al. (Impact of alg3 gene deletion on growth, development, pigment production, protein secretion, and functions of recombinant Trichoderma reesei cellobiohydrolases in Aspergillus niger, Fungal Genetics and Biology, 2013, Vol. 61, pages 120-132).

As also shown in FIG. 2, the GSA202 products from the L137F mutant and the R15* mutant showed shifted molecular weight, meaning that the products were less glycosylated than the products from the wildtype strain. The major peaks of GSA202 from the L137F mutant and the R15* mutant were very consistent, wherein the difference between GSA202 from the wildtype and GSA202 from the L137F mutant and the R15* mutant presumably corresponds to 12 hexose units.

However, unlike GSA202 from the R15* mutant, the GSA202 products from the L137F mutant showed additional sub-peaks which correspond to additional mannose units (indicated by the double arrow in FIG. 2). Together with the 8.6% increase of relative GSA202 activity of the L137F mutant shown in Table 5 of Example 3, the MS data of FIG. 2 indicates that the L137F mutation does not disrupt alg3 function, as is the case for the R15* mutant, but instead alters the alg3 function, which may be beneficial to higher GSA202 production. It is further noted, as shown in Table 7, that the different glycosylation patterns obtained from the different alg3 mutants do not affect the specific activity of GSA202.

It was completely surprising and unexpected that alg3 mutants T17I and L137F did not result in disrupted alg3 function and activity as shown for the R15* mutant, but instead achieved increased product yield/relative activity with unaltered alg3 function (T17I=identical product glycosylation to WT) and altered alg3 function (L137F products with additional mannose units). It was furthermore surprising that alg3 deletion is not necessary to obtain increased product yield and that increased product yield can also be achieved by AA substitutions at position T17 and/or L137. In particular, the L137F mutant is advantageous when it is intended to produce a protein of interest having a slightly modified N-glycan profile, i.e. an N-glycan profile comprising less complex and smaller N-glycans, yet with added mannose structures as shown in FIG. 2.

Alg3 function includes the addition of mannose structures on N-glycans during N-glycan processing. Reduced alg3 function is therefore associated with a lower abundance of high-mannose N-glycan structures, which are well-known to cause immunogenicity in humans. Advantageously, the altered glycosylation towards less high-mannose N-glycans of the product caused by L137F mutations respectively, while in parallel leading to increased product yields, is suggested to contribute to reduced immunogenicity of the protein product which might be caused by high-mannose N-glycans. On the other hand, and in contrast to the relatively small N-glycans of the alg3-knockout mutant R15*, the larger N-glycan portions of mutants L137F and T17I are likely to increase stability and/or solubility of the protein product while also showing increased product yields compared to the wildtype.

TABLE 7
Normalized specific activity of GSA202
from A. niger alg3 mutants

| alg3 | Normalized specific activity | Glycosylation pattern |
|---|---|---|
| mutation | of GSA202 (WT = 100%) | (see FIG. 2) |
| L137F | 101.72% +/− 7.73% | Low + additional |
| | | mannose peaks |
| T17I | 102.33% +/− 2.21% | High |
| WT | 100% +/− 0.122% | High |

### Example 5: Genomic DNA Extraction from Trichoderma reesei

Trichoderma reesei was grown in 50 ml of YPG medium in a 250 ml baffled shake flask at 28° C. for 2 days with agitation at 200 rpm. Mycelia from the cultivation was collected using a MIRACLOTH® (EMD Chemicals Inc.) lined funnel, squeeze-dried, and then transferred to a pre-chilled mortar and pestle. Each mycelia preparation was ground into a fine powder and kept frozen with liquid nitrogen. A total of 1-2 g of powder was transferred to a 50 ml tube and genomic DNA was extracted from the ground mycelial powder using a DNEASY® Plant Maxi Kit (QIAGEN Inc.). Five ml of Buffer AP1 (QIAGEN Inc.) pre-heated to 65° C. was added to the 50 ml tube followed by 10 μl of RNase A 100 mg/ml stock solution (QIAGEN Inc.) and incubated for 2-3 hours at 65° C. A total of 1.8 ml of AP2 Buffer (QIAGEN Inc.) was added and centrifuged at 3000-5000×g for 5 minutes. The supernatant was decanted into a QIAshredder Maxi Spin Column (QIAGEN Inc.) placed in a 50 ml collection tube, and centrifuged at 3000-5000×g for 5 minutes at room temperature (15-25° C.) in a swing-out rotor. The flow-through in the collection tube was transferred, without disturbing the pellet, into a new 50 ml tube. A 1.5 ml volume of Buffer AP3/E (QIAGEN Inc.) was added to the cleared lysate, and mixed immediately by vortexing. The sample (maximum 15 ml), including any precipitate that may form, was pipetted into a DNEASY® Maxi Spin Column (QIAGEN Inc.) placed in a 50 ml collection tube and centrifuged at 3000-5000×g for 5 minutes at room temperature (15-25° C.) in a swing-out rotor. The flow-through was discarded. Twelve ml of Buffer AW (QIAGEN Inc.) was added to the DNEASY® Maxi Spin Column, and centrifuged for 10 minutes at 3000-5000×g to dry the membrane. The flow-through and collection tube were discarded. The DNEASY® Maxi Spin Column was transferred to a new 50 ml tube. One-half ml of Buffer AE (QIAGEN Inc.), pre-heated to 65° C., was pipetted directly onto the DNEASY® Maxi Spin Column membrane, incubated for 5 minutes at room temperature (15-25° C.), and then centrifuged for 5 minutes at 3000-5000×g to elute the genomic DNA. The concentration and purity of the genomic DNA was determined by measuring the absorbance at 260 nm and 280 nm.

### Example 6: Trichoderma reesei Protoplast Generation

Protoplast preparation and transformation of Trichoderma reeseiwere performed using a protocol similar to Penttila et al., 1987, Gene 61: 155-164. Briefly, T. reesei was cultivated in two shake flasks, each containing 25 ml of YPG medium, at 30° C. for 16 hours with gentle agitation at 90 rpm. Mycelia were collected by filtration using a Vacuum Driven Disposable Filtration System (Millipore) and washed twice with deionized water and twice with 1.2 M sorbitol. Protoplasts were generated by suspending the washed mycelia in 30 ml of 1.2 M sorbitol containing 5 mg/ml of Yatalase™ (Takara Bio USA, Inc.) and 0.5 mg/ml of Chitinase (Sigma Chemical Co.) 15-25 minutes (or until protoplasts were present) at 34° C. with gentle shaking at 90 rpm. Protoplasts were collected by centrifugation at 834×g for 7 minutes and washed twice with cold 1.2 M sorbitol. The protoplasts were counted using a hemocytometer and re-suspended to a final concentration of 1×10⁸ protoplasts per ml of Tr-STC.

### Example 7: Alignment of A. niger Alg3 to T. reesei Alg3

An alignment of the T. reesei Alg3 protein (SEQ ID. NO: 21) to the A. nigerAlg3 protein (SEQ ID. NO: 7) was performed to identify the positions in T. reesei Alg3 protein, which correspond to the S17 and L137 positions where mutation was found to be beneficial for enzyme expression in A. niger. The proteins were aligned using the MUSCLE algorithm version 3.8.31 with default parameters (Edgar, R. C. (2004). Nucleic Acids Research, 32(5), 1792-1797).

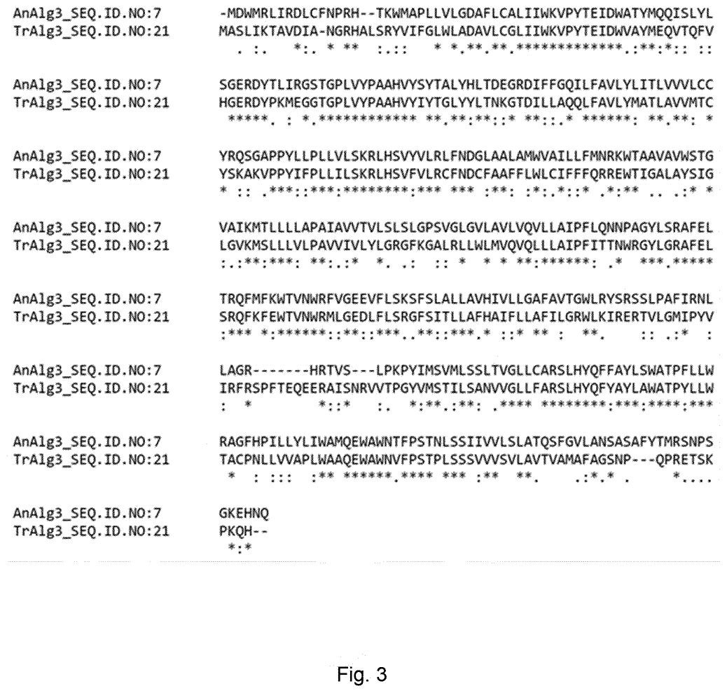

The results from this sequence alignment are shown in FIG. 3. The sequence identity between the wildtype Alg3 polypeptides of T. reesei (SEQ ID NO: 21) and A. niger(SEQ ID NO: 3) has been shown to be 52.4%. Based on the findings of below examples the Alg3 polypeptide appears to perform a highly conserved and similar function across different filamentous fungal host cells. Based on the alignment, it was concluded that the L137F mutation in A. niger correspond to L139F in T. reesei. The T17I A. niger mutation was not as obvious since the N-terminal portion of the molecules are not as well conserved as is the region around the L137F mutation. However, it was concluded that T17I A. niger mutation would correspond to S19I in T. reesei. Consequently, it was decided to test how a S19I or a L139F mutation in T. reesei Alg3 would affect enzyme product yield in T. reesei.

### Example 8: Nuclease Backbone Vector pGMEr280

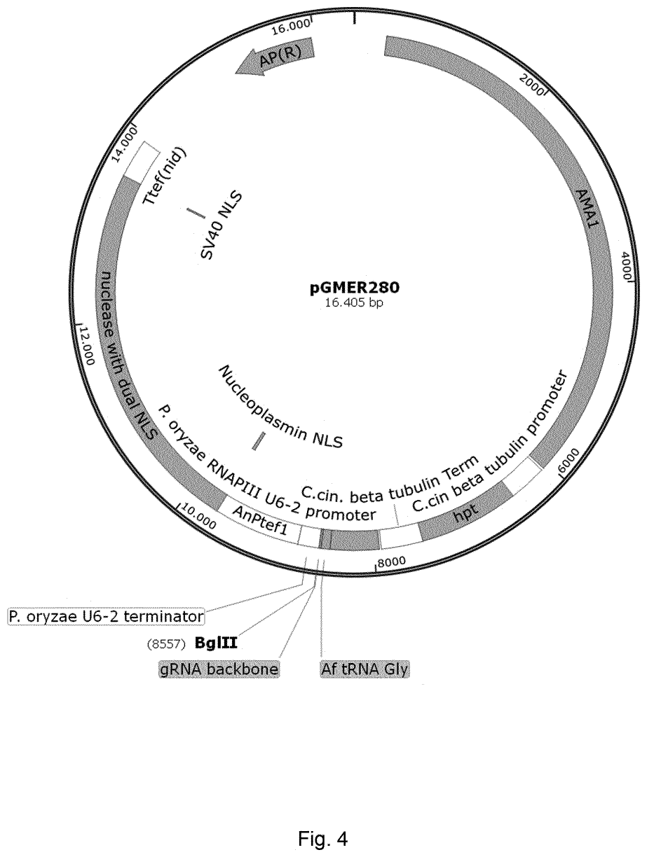

Plasmid pGMEr280 (FIG. 4) is a single RNA-guided DNA endonuclease expression plasmid used to clone in protospacers into BglII digested pGMEr280 using an NEBuilder® HiFi DNA Assembly Cloning Kit (New England Biolabs Inc.). Plasmid pGMEr280 contains a single RNA-quided DNA endonuclease protein coding sequence (codon-optimized for use in Aspergillus oryzae) with an N-terminal nucleoplasmin nuclear localization signal and a C-terminal SV40 nuclear localization signal to ensure that the nuclease would be localized to the nucleus.

The expression of the nuclease is under control of the Aspergillus nidulans tef1 promoter and terminator from pFC330-333 (Nødvig et al., 2015, PLoS One 10(7): 1-18). Plasmid pGMEr280 also has all the elements for single guide RNA (sgRNA) expression, which consists of the Magnaporthe oryzae U6-2 promoter, Aspergillus fumigatus tRNAgly(GCC)1-6 sequence with the region downstream the structural tRNA removed, single guide RNA sequence, BglII restriction enzyme recognition sequence, and M. oryzae U6-2 terminator. For selection in T. reesei, plasmid pGMEr280 contains the hygromycin phosphotransferase gene from pHT1 (Cummings et al., 1999, Curr. Genet. 36: 371), conferring resistance to hygromycin B, and the autonomous maintenance in Aspergillus (AMA1) sequence (Gems et al., 1991, Gene 98: 61-67) for extrachromosomal replication of pGMEr280 in T. reesei. The single guide RNA and the Nucleoplasmin-Nuclease-SV40 NLS expression elements in pGMEr280 were confirmed by DNA sequencing using 2×150 bp chemistry on a NEXTSEQ™ 500 system (Illumina Inc.).

### Example 9: Construction of pAgJg341 and pAgJg342 Nuclease Plasmids to Target alg3 in T. reesei

Plasmid vector preparation. Plasmid pGMEr280 was digested with the restriction enzyme BglII (Anza™ 19 BglII, Thermo Fisher Scientific). The restriction reaction contained: 15 μg of pGMEr280 plasmid DNA, 1× Anza™ buffer, 100 units of BglII, and sterile Milli-Q water up to 200 μl final volume. The reaction was incubated at 37° C. for 3 hours. Following restriction enzyme digestion, the digest was subjected to 0.8% agarose gel electrophoresis in TBE buffer and the band representing the digested pGMEr280 was excised from the gel and purified using a NucleoSpin® Gel and PCR Clean-up kit (Macherey-Nagel) according to the manufacturer's instructions.

Protospacer design. Two protospacers were designed to direct the nuclease to the target site and create a double stranded break in the alg3 gene, one for position S17 in the Alg3 protein and one for position L139 in Alg3 (SEQ ID NO: 22 and SEQ ID NO: 23). Protospacers were selected by finding an appropriate protospacer adjacent motif (PAM) with the sequence TTTV, where V represents nucleotides A, C, or G. Once an appropriate PAM site was identified, the twenty-one base-pairs immediately adjacent to the 3′ side of the PAM site were selected as the protospacer. Protospacers that contained more than three contiguous T nucleotides were rejected to avoid possible stuttering of RNA polymerase.

Each protospacer with its extension sequences used for cloning (U.S. Pat. Nos. 1,238,937, 1,238,938) was synthesized as a single-stranded oligonucleotide by Thermo Fisher Scientific, Inc. All protospacer oligonucleotides were diluted to a final working concentration of 5 pmol/μl.

Assembly of protospacers. Protospacers were cloned into pGMEr280 using an NEBuilder® HiFi DNA Assembly Master Mix kit (New England Biolabs) in a total volume of 10 μl composed of 1× NEBuilder® HiFi Assembly Master Mix, 50-100 ng of BglII-digested pGMEr280, 1.0 μl of protospacer oligo (5 pmol) and sterile Milli-Q H₂O to a final volume of 10 μl. The reactions were incubated at 50° C. for 15 minutes and then placed on ice. Two μL of each reaction was used to transform 50 μL Stellar™ Competent Cells (Clontech Laboratories, Inc.) according to the manufacturer's instructions. Each transformation reaction was spread onto two LB+Amp plates and incubated at 37° C. overnight. Putative transformant colonies were isolated from the selection plates and plasmid DNA was prepared from each one using a KingFisher Duo platform with the KingFisher Pure Plasmid Kit (Thermo Fisher Scientific Inc). Plasmid DNA was sequenced on an Oxford Nanopore ONT Sequencing platform. Plasmids having the correct protospacer sequence were labelled pAgJg341 (to make the cut for the Alg3 S19I mutation) and pAgJg342 (to make the cut for the Alg3 L139F mutation). The protospacer present in pAgJg341 and pAgJg342 can be found as SEQ ID NO: 24 and SEQ ID NO: 25, respectively.

### Example 10: Construction of T. reesei Alg3 S19I Mutant

Trichoderma reesei 6Q-M1002 protoplasts were generated as described in example 6. Prior to transformation, a double-stranded oligo containing the desired mutation including a mutation intended to change the nuclease PAM site to prevent re-cutting upon repair was prepared by annealing the complementary oligos 1238939 and 1238940 together.

Oligos were reconstituted to a concentration of 50 pmol/μl. Equal amounts of each oligo (100 μl each) were added together and heated at 95° C. for 5 minutes. Then the tubes were moved to room temperature where the tubes slowly came down in temperature.

Approximately 12.5 μg of pAgJg341 nuclease plasmid+3000 pmol (60 μl) of dsDNA oligo (described above) to serve as repair template was added to 500 μl of protoplast solution and mixed gently. PEG buffer (1250 μl) was added, and the reaction was mixed and incubated at 37° C. for 30 minutes. Tr-STC (5 ml) was then added and the contents were spread onto PDA+1 M sucrose plates and incubated overnight at 32° C. The next day, an overlay consisting of PDA+hygromycin B was added to a final concentration of 10 μg/ml hygromycin B and the plates were incubated at 30° C. for 7 days. Next, hygromycin resistant transformants were transferred to COVE2 plates and incubated at 30° C. for 5-7 days. Transformants were screened for correct modification of the alg3 locus by spore PCR and Oxford Nanopore ONT Sequencing. For each transformant, spores were collected with a sterile 1 μl inoculation loop and suspended in 20 μl of Dilution buffer (PHIRE™ Plant Direct PCR Kit, Thermo Scientific) in a thin-walled PCR tube. Each spore suspension was used as template in a PCR reaction to screen for correct modification of the alg3 locus (SEQ ID NO: 28). Primers 1238985 (SEQ ID NO: 29) & 1238986 (SEQ ID NO:30) were used to amplify part of the alg3 locus containing the intended mutations.

Each PCR reaction was composed of 1 μl of spore suspension, 20 pmol of each primer, 10 μl of 2× PHIRE™ Plant PCR Buffer (PHIRE™ Plant Direct PCR Kit, Thermo Scientific), 0.4 μl of PHIRE™ Hot Start II DNA Polymerase (PHIRE™ Plant Direct PCR Kit, Thermo Scientific) and H₂O to a final volume of 20 μl. Thermocycling was performed according the manufacturer's instructions. The PCR products were analyzed by 1% agarose gel electrophoresis using TBE buffer. The PCRs were cleaned up with ExoSaplt. Five μl of the PCR reaction was added to 2 μl of ExoSaplt. The reaction was incubated at 37° C. for 15 minutes, then 80° C. for 15 minutes. DNA was quantified on a Qubit fluorometer and submitted for Oxford Nanopore ONT sequencing. The results were analyzed, and spores were picked from COVE2 plates from two of the transformants (M1002-S19I-1 and M1002-S19I-5) containing the desired mutations and subjected to spore isolation by plating dilutions onto PDA+1 M sucrose plates. The plates were incubated at 30° C. for 3 days. Spore isolates M1002-S19I-1A and M1002-S19I-5A were subcultured onto COVE2 plates and incubated at 30° C. Next, the spore isolates from were subjected to a second round of spore isolation by plating dilutions onto PDA+1 M sucrose plates and incubation at 30° C. for 3 days. Spore isolates M1002-S19I-1A1 and M1002-S19I-5A1 were subcultured onto PDA+1 M sucrose plates. Genomic DNA was prepared from each according to example 5 and sent for Oxford Nanopore ONT Sequencing. The isolates contained the desired modification and were saved as M1002-S19I-1A1 and M1002-S19I-5A1 for further studies.

### Example 11: Construction of T. reesei Alg3 L139F Mutant

Trichoderma reesei 6Q-M1002 protoplasts were generated as described in example 6. Prior to transformation, a double-stranded oligo containing the desired mutation including a mutation intended to change the nuclease PAM site to prevent re-cutting upon repair was prepared by annealing the complementary oligos 1238941 (SEQ ID NO: 31) and 1238942 (SEQ ID NO: 32) together.

Oligos were reconstituted to a concentration of 50 pmol/μl. Equal amounts of each oligo (100 μl each) were added together and heated at 95° C. for 5 minutes. Then the tubes were moved to room temperature where the tubes slowly came down in temperature.

Approximately 12.5 μg of pAgJg342 nuclease plasmid+3000 pmol (60 μl) of dsDNA oligo (described above) to serve as repair template was added to 500 μl of protoplast solution and mixed gently. PEG buffer (1250 μl) was added, and the reaction was mixed and incubated at 37° C. for 30 minutes. Tr-STC (5 ml) was then added and the contents were spread onto PDA+1 M sucrose plates and incubated overnight at 32° C. The next day, an overlay consisting of PDA+hygromycin B was added to a final concentration of 10 μg/ml hygromycin B and the plates were incubated at 30° C. for 7 days. Next, hygromycin resistant transformants were transferred to COVE2 plates and incubated at 30° C. for 5-7 days. Transformants were screened for correct modification of the alg3 locus by spore PCR and Oxford Nanopore ONT Sequencing. For each transformant, spores were collected with a sterile 1 μl inoculation loop and suspended in 20 μl of Dilution buffer (PHIRE™ Plant Direct PCR Kit, Thermo Scientific) in a thin-walled PCR tube. Each spore suspension was used as template in a PCR reaction to screen for correct modification of the alg3 locus. Primers 1238985 (SEQ ID NO: 29) & 1238986 (SEQ ID NO: 30) were used to amplify part of the alg3 locus containing the intended mutations. Each PCR reaction was composed of 1 μl of spore suspension, 20 pmol of each primer, 10 μl of 2× PHIRE™ Plant PCR Buffer (PHIRE™ Plant Direct PCR Kit, Thermo Scientific), 0.4 μl of PHIRE™ Hot Start II DNA Polymerase (PHIRE™ Plant Direct PCR Kit, Thermo Scientific) and H₂O to a final volume of 20 μl. Thermocycling was performed according the manufacturer's instructions. The PCR products were analyzed by 1% agarose gel electrophoresis using TBE buffer. The PCRs were cleaned up with ExoSaplt. Five μl of the PCR reaction was added to 2 μl of ExoSaplt. The reaction was incubated at 37° C. for 15 minutes, then 80° C. for 15 minutes. DNA was quantified on a Qubit fluorometer and submitted for Oxford Nanopore ONT sequencing. The results were analyzed, and spores were picked from COVE2 plates from two of the transformants (M1002-L139F-1 and M1002-L139F-3) containing the desired mutations and subjected to spore isolation by plating dilutions onto PDA+1 M sucrose plates. The plates were incubated at 30° C. for 3 days. Spore isolates M1002-L139F-1A and M1002-L139F-3C were subcultured onto COVE2 plates and incubated at 30° C. Next, the spore isolates from were subjected to a second round of spore isolation by plating dilutions onto PDA+1 M sucrose plates and incubation at 30° C. for 3 days. Spore isolates M1002-L139F-1A1 and M1002-L139F-3C1 were subcultured onto PDA+1 M sucrose plates. Genomic DNA was prepared from each according to example 5 and sent for Oxford Nanopore ONT Sequencing. The isolates contained the desired modification and were saved as M1002-L139F-1A1 and M1002-L139F-3C1 for further studies.

### Example 12: Lysozyme Activity Assay (LSU(F)/ml)

Whole broth from fermentation was mixed for roughly 2 hours in a rotisserie mixer at 30° C. After whole broth mixing, all samples were diluted 100× in pre-dilution buffer, then mixed for roughly 2 hours using the rotisserie mixer again. Next, the 100× pre-diluted samples were diluted 10000× in 0.1 M Tris-HCl, 0.1M NaCl, 0.01% Triton X-100 buffer pH 7.5 (sample buffer) by 10-fold serial dilutions followed with a series of 3× dilutions down to 1/27 of the diluted sample. This method was used in conjunction with a Beckman Coulter Biomek FX and SpectraMax plate reader from Molecular Devices. A lysozyme standard was diluted from 0.05 LSU(F)/ml concentration and ending with a 0.002 LSU(F)/ml concentration in the sample buffer. A total of 50 μl of each dilution including standard was transferred to a 96-well flat bottom plate. Fifty micro-liters of a 25 ug/ml fluorescein-conjugated cell walls substrate solution was added to each well then incubated at ambient temperature for 45 minutes. During the incubation, the rate of the reaction was monitored at 485 nm (excitation)/528 nm (emission) for the 96-well plate at 15-minute intervals. Sample concentrations were determined by extrapolation from the generated standard curve.

### Example 13: Lab-Scale Fermentation Showed that Mutations S19I and L139F in T. reesei Alg3 Leads to Increased Lysozyme Productivity/Yield

The alg3 mutant strains M1002-S19I-1A1 and M1002-L139F-1A1 strains were evaluated in 2 liter fermentations along with the control strain 6Q-M1002 according to the protocol mentioned in example 12 in WO 2020/123845. Aliquots of whole broth were taken on day 7 and stored at 5 to 10° C. until they were processed for lysozyme activity assay.

The lysozyme expression level was determined as described in Example 12. As seen in table 8 the S19I and L139F mutations in T. reesei Alg3 lead to a 7-8% increase in lysozyme titer compared to the 6Q-M1002 strain, which expresses an unmodified Alg3 protein.

Concluding, above results confirm that Alg3 mutations at position S19 (relating to T17 in A. niger) and at position L139 (relating to L137 in A. niger) contribute to increased enzyme yield during the production of different classes of enzymes, such as lysozyme enzymes and glucoamylase enzymes. As shown throughout the examples, the positive effects of said mutations are not limited to one class of enzymes or one type of fungal species only, and are therefore expected to work for other fungal host cells too which comprise an Alg3-pathway.

TABLE 8
Lysozyme activity/yield of wildtype alg3
T. reesei strains and alg3 mutants.

| Strain | Alg3 mutation | LSU(F) relative activity |
|---|---|---|
| 6Q-M1002 | None | 1.00 |
| M1002-S19I-1A1 | S19I | 1.07 |
| M1002-L139F-1A1 | L139F | 1.08 |

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

The invention is further defined by the following numbered paragraphs:

1. A fungal host cell comprising in its genome:
a first polynucleotide encoding a polypeptide of interest; and
a second polynucleotide encoding a polypeptide having a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100% to SEQ ID NO: 7 and comprising an alteration at a position corresponding to position 15, 17 and/or 137 of SEQ ID NO: 7.

2. The fungal host cell according to paragraph 1, wherein the alteration of said polypeptide at the position corresponding to position 15, 17 and/or 137 of SEQ ID NO: 7 is, independently chosen from one another, an amino acid substitution, an amino acid insertion, an amino acid deletion or a premature polypeptide termination.

3. The fungal host cell according to any one of paragraphs 1-2, wherein the alteration of said polypeptide at the position corresponding to position 137 of SEQ ID NO: 7 is an amino acid substitution, preferably a substitution of leucine by phenylalanine, L137F, as presented in SEQ ID NO: 8.

4. The fungal host cell according to any one of paragraphs 1-2, wherein the alteration of said polypeptide at the position corresponding to position 17 of SEQ ID NO: 7 is an amino acid substitution, preferably a substitution of threonine by isoleucine, T17I, as presented in SEQ ID NO: 18.

5. The fungal host cell according to any one of paragraphs 1-2, wherein the alteration of said polypeptide at the position corresponding to position 15 of SEQ ID NO: 7 is a premature polypeptide termination R15* as presented in SEQ ID NO: 15.

6. The fungal host cell according to any one of paragraphs 1-2, wherein the alteration of said polypeptide at the position corresponding to position 137 of SEQ ID NO: 7 is an amino acid substitution, such as a substitution of leucine by isoleucine, valine, histidine, lysine, methionine, threonine, tryptophan, asparagine, aspartic acid/aspartate, alanine, arginine, cysteine/cystine, glutamine, glutamic acid/glutamate, glycine, proline, serine, or tyrosine.

7. The fungal host cell according to any one of paragraphs 1-2, wherein the alteration of said polypeptide at the position corresponding to position 17 of SEQ ID NO: 7 is an amino acid substitution, such as a substitution of threonine by isoleucine, valine, histidine, lysine, methionine, leucine, tryptophan, asparagine, aspartic acid/aspartate, alanine, arginine, cysteine/cystine, glutamine, glutamic acid/glutamate, glycine, proline, serine, or tyrosine.

8. The fungal host cell according to any one of paragraphs 1-2, wherein the alteration of said polypeptide at the position corresponding to position 15, 17 and/or 137 of SEQ ID NO: 7 is an amino acid insertion, such as an amino acid insertion of at least one amino acid selected from the list of leucine, phenylalanine, isoleucine, valine, histidine, lysine, methionine, threonine, tryptophan, asparagine, aspartic acid/aspartate, alanine, arginine, cysteine/cystine, glutamine, glutamic acid/glutamate, glycine, proline, serine, or tyrosine, wherein the amino acid insertion can be an insertion of one, two, three, four, five, or more than five amino acids, wherein the amino acids are independently chosen from one another.

9. A fungal host cell comprising in its genome:
a first polynucleotide encoding a polypeptide of interest; and
a second polynucleotide encoding a polypeptide having a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100% to SEQ ID NO: 21 and comprising an alteration at a position corresponding to position 19 and/or 139 of SEQ ID NO: 21.

10. The fungal host cell according to paragraph 9, wherein the alteration of said polypeptide at the position corresponding to position 19 and/or 139 of SEQ ID NO: 21 is, independently chosen from one another, an amino acid substitution, an amino acid insertion, an amino acid deletion or a premature polypeptide termination.

11. The fungal host cell according to any one of paragraphs 9-10, wherein the alteration of said polypeptide at the position corresponding to position 139 of SEQ ID NO: 21 is an amino acid substitution, preferably a substitution of leucine by phenylalanine, L139F, as presented in SEQ ID NO: 39.

12. The fungal host cell according to any one of paragraphs 9-10, wherein the alteration of said polypeptide at the position corresponding to position 19 of SEQ ID NO: 21 is an amino acid substitution, preferably a substitution of serine by isoleucine, S19I, as presented in SEQ ID NO: 36.

13. The fungal host cell according to any one of paragraphs 9-10, wherein the alteration of said polypeptide at the position corresponding to position 139 of SEQ ID NO: 21 is an amino acid substitution, such as a substitution of leucine by isoleucine, valine, histidine, lysine, methionine, threonine, tryptophan, asparagine, aspartic acid/aspartate, alanine, arginine, cysteine/cystine, glutamine, glutamic acid/glutamate, glycine, proline, serine, or tyrosine.

14. The fungal host cell according to any one of paragraphs 9-10, wherein the alteration of said polypeptide at the position corresponding to position 19 of SEQ ID NO: 21 is an amino acid substitution, such as a substitution of threonine by isoleucine, valine, histidine, lysine, methionine, leucine, tryptophan, asparagine, aspartic acid/aspartate, alanine, arginine, cysteine/cystine, glutamine, glutamic acid/glutamate, glycine, proline, serine, or tyrosine.

15. The fungal host cell according to any one of paragraphs 9-10, wherein the alteration of said polypeptide at the position corresponding to position 19 and/or 139 of SEQ ID NO: 21 is an amino acid insertion, such as an amino acid insertion of at least one amino acid selected from the list of leucine, phenylalanine, isoleucine, valine, histidine, lysine, methionine, threonine, tryptophan, asparagine, aspartic acid/aspartate, alanine, arginine, cysteine/cystine, glutamine, glutamic acid/glutamate, glycine, proline, serine, or tyrosine, wherein the amino acid insertion can be an insertion of one, two, three, four, five, or more than five amino acids, wherein the amino acids are independently chosen from one another.

16. The fungal host cell according to any one of paragraphs 1-15, wherein the fungal host cell is a yeast host cell; preferably the yeast host cell is selected from the group consisting of Candida, Hansenula, Kluyveromyces, Pichia (Komagataella), Saccharomyces, Schizosaccharomyces, and Yarrowia cell; more preferably the yeast host cell is selected from the group consisting of Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, and Yarrowia lipolytica cell, most preferably Pichia pastoris (Komagataella phaffii).

17. The fungal host cell according to any one of paragraphs 1-15, wherein the fungal host cell is a filamentous fungal host cell; preferably the filamentous fungal host cell is selected from the group consisting of Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, and Trichoderma cell; more preferably the filamentous fungal host cell is selected from the group consisting of Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, and Trichoderma viride cell; even more preferably the filamentous host cell is selected from the group consisting of Aspergillus oryzae, Fusarium venenatum, and Trichoderma reesei cell; most preferably the filamentous fungal host cell is an Aspergillus niger cell.

18. The fungal host cell according to any one of paragraphs 1-15, wherein the fungal host cell is an Aspergillus oryzae cell.

19. The fungal host cell according to any one of paragraphs 1-15, wherein the fungal host cell is a Trichoderma reesei cell.

20. The fungal host cell according to any one of paragraphs 1-19, wherein the polypeptide of interest comprises an enzyme; preferably the enzyme is selected from the group consisting of hydrolase, isomerase, ligase, lyase, oxidoreductase, or transferase; more preferably an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, alpha-glucosidase, beta-glucosidase, invertase, laccase, lipase, mannosidase, mutanase, nuclease, oxidase, pectinolytic enzyme, peroxidase, phosphodiesterase, phytase, polyphenoloxidase, proteolytic enzyme, ribonuclease, transglutaminase, xylanase, and beta-xylosidase.

21. The fungal host cell according to any one of paragraphs 1-19, wherein the polypeptide of interest is a glycoprotein.

22. The fungal host cell according to any one of paragraphs 1-20, wherein the polypeptide of interest is an alpha-glucosidase, preferably an 1,4-alpha-glucosidase, most preferably a glucoamylase.

23. The fungal host cell according to any one of paragraphs 1-22, wherein the polypeptide of interest comprises, consists essentially of, or consists of SEQ ID NO: 9.

24. The fungal host cell according to any one of paragraphs 1-22, wherein the polypeptide of interest comprises, consists essentially of, or consists of SEQ ID NO: 11.

25. The fungal host cell according to any one of paragraphs 1-19, wherein the polypeptide of interest is a hydrolase, preferably a glycoside hydrolase.

26. The fungal host cell according to paragraph 25, wherein the polypeptide of interest is a lysozyme, preferably a lysozyme which comprises, consists essentially of, or consists of SEQ ID NO: 33.

27. The fungal host cell according to any one of paragraphs 1-26, wherein the host cell comprises two or more copies of the first polynucleotide.

28. The fungal host cell according to any one of paragraphs 1-27, wherein the alteration of the polypeptide at the position corresponding to position 137 of SEQ ID NO: 7, is resulting from a single nucleotide polymorphism (SNP) within the second polynucleotide, preferably the SNP within the second polynucleotide is an alteration at a position corresponding to position 496, 496 and/or 497 of the second polynucleotide with SEQ ID NO: 3, wherein the second polynucleotide is a polynucleotide variant having a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100%, to SEQ ID NO: 3.

29. The fungal host cell according to any one of paragraphs 1-27, wherein the alteration of the polypeptide at the position corresponding to position 17 of SEQ ID NO: 7, is resulting from a single nucleotide polymorphism (SNP) within the second polynucleotide, preferably the SNP within the second polynucleotide is an alteration at a position corresponding to position 49, 50 and/or 51 of the second polynucleotide with SEQ ID NO: 3, wherein the second polynucleotide is a polynucleotide variant having a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100%, to SEQ ID NO: 3.

30. The fungal host cell according to any one of paragraphs 1-27, wherein the alteration of the polypeptide at the position corresponding to position 15 of SEQ ID NO: 7, is resulting from a single nucleotide polymorphism (SNP) within the second polynucleotide, preferably the SNP within the second polynucleotide is an alteration at a position corresponding to position 43, 44 and/or 45 of the second polynucleotide with SEQ ID NO: 3, wherein the second polynucleotide is a polynucleotide variant having a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100%, to SEQ ID NO: 3, which SNP is leading to a premature stop codon.

31. A method of producing one or more polypeptide of interest, comprising cultivating a cell according to any one of paragraphs 1-30 under conditions conducive for production of the one or more polypeptide of interest.

32. The method according to paragraph 31, wherein the polypeptide of interest optionally is recovered.

33. A nucleic acid construct comprising a heterologous promoter operably linked to a polynucleotide encoding a polypeptide having a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100% to SEQ ID NO: 7 and comprising an alteration at a position corresponding to position 15, 17 or 137 of SEQ ID NO: 7.

34. The nucleic acid construct according to paragraph 33, wherein the alteration at the position corresponding to position 137 of SEQ ID NO: 7 is a substitution; preferably a substitution of leucine for phenylalanine, L137F according to SEQ ID NO 8.

35. The nucleic acid construct according to paragraph 33, wherein the alteration at the position corresponding to position 17 of SEQ ID NO: 7 is an amino acid substitution, preferably a substitution of threonine by isoleucine, T17I, as presented in SEQ ID NO: 18.

36. The nucleic acid construct according to paragraph 33, wherein the alteration at the position corresponding to position 15 of SEQ ID NO: 7 is a premature polypeptide termination R15* as presented in SEQ ID NO: 15.

37. The nucleic acid construct according to paragraph 33, wherein the alteration at the position corresponding to position 15 or 137 of SEQ ID NO: 7 comprises or consists of an alteration, preferably a substitution, wherein the variant has a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, to the amino acid sequence of SEQ ID NO: 7.

38. The nucleic acid construct according to paragraph 34, wherein the amino acid at a position corresponding to position 137 of SEQ ID NO: 7 is substituted with Ala, Arg, Asn, Asp, Cys, Gln, Glu, Gly, His, lie, Leu, Lys, Met, Pro, Ser, Thr, Trp, Tyr, or Val, preferably with Phe.

39. The nucleic acid construct according to paragraph 35, wherein the amino acid at a position corresponding to position 17 of SEQ ID NO: 7 is substituted with Ala, Arg, Asn, Asp, Cys, Gln, Glu, Gly, His, Phe, Leu, Lys, Met, Pro, Ser, Thr, Trp, Tyr, or Val, preferably with Ile.

40. The nucleic acid construct according to paragraph 36, wherein the polypeptide has a length of 14 amino acids or less, said polypeptide with 14 amino acids or less has a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, preferably 100%, to the amino acid sequence of SEQ ID NO: 15.

41. The nucleic acid construct according to paragraph 33, wherein the alteration of said polypeptide at the position corresponding to position 13, 15 or 137 of SEQ ID NO: 7 is an amino acid insertion, such as an amino acid insertion of at least one amino acid selected from the list of leucine, phenylalanine, isoleucine, valine, histidine, lysine, methionine, threonine, tryptophan, asparagine, aspartic acid/aspartate, alanine, arginine, cysteine/cystine, glutamine, glutamic acid/glutamate, glycine, proline, serine, or tyrosine.

42. The nucleic acid construct according to paragraph 41, wherein the amino acid insertion is an insertion of one, two, three, four, five, or more than five amino acids, wherein the amino acids are independently chosen from one another.

43. The nucleic acid construct according to any one of paragraphs 33-42, wherein the polynucleotide encoding the polypeptide has a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100% to SEQ ID NO: 3 or SEQ ID NO: 4.

44. The nucleic acid construct according to any one of paragraphs 33-43, wherein the polynucleotide encoding the polypeptide has a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100% to SEQ ID NO: 3 and comprises at least one nucleotide alteration at a position corresponding to position 43, 44, 45, 49, 50, 51, 495, 496 and/or 497 of SEQ ID NO: 3.

45. The nucleic acid construct according to any one of paragraphs 33-43, wherein the polynucleotide encoding the polypeptide has a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100% to SEQ ID NO: 3 and comprises at least one nucleotide alteration at a position corresponding to position 43, 44, 45, 49, 50, and/or 51 of SEQ ID NO: 4.

46. The nucleic acid construct according to paragraph 43, wherein the at least one nucleotide alteration is a substitution; preferably the nucleotide(s) at a position corresponding to position 495, 496, and/or 497 of SEQ ID NO: 3 is/are independently from another substituted with adenine (A), thymine (T), guanine (G) and/or cytosine (C).

47. The nucleic acid construct according to paragraph 46, wherein the at least one nucleotide alteration comprises or consists of a substitution of cytosine with thymine at a position corresponding to position 495 of SEQ ID NO: 3, C495T, as shown in SEQ ID NO: 5.

48. The nucleic acid construct according to paragraph 46, wherein the at least one nucleotide alteration comprises or consists of a substitution of cytosine with thymine at a position corresponding to position 495 of SEQ ID NO: 3, C495T, and of a substitution of thymine with cytosine at a position corresponding to position 497 of SEQ ID NO: 3.

49. The nucleic acid construct according to paragraph 46, wherein one, two or three of the nucleotides corresponding to position 495, 496 and/or 497 of SEQ ID NO: 3 are independently from another substituted with adenine (A), thymine (T), guanine (G) or cytosine (C).

50. The nucleic acid construct according to paragraph 46, wherein one, two or three of the nucleotides corresponding to position 495, 496 and/or 497 of SEQ ID NO: 3 are, independently from another, deleted.

51. The nucleic acid construct according to any one of paragraphs 33-43, wherein the polynucleotide encoding the polypeptide has a sequence identity of at least 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100% to SEQ ID NO: 4 and comprises at least one nucleotide alteration at a position corresponding to position 409, 410 and/or 411 of SEQ ID NO: 4.

52. The nucleic acid construct according to paragraph 51, wherein the at least one nucleotide alteration is a substitution; preferably the nucleotide(s) at a position corresponding to position 409, 410, and/or 411 of SEQ ID NO: 4 is/are independently from another substituted with adenine (A), thymine (T), guanine (G) and/or cytosine (C).

53. The nucleic acid construct according to paragraph 51, wherein the at least one nucleotide alteration comprises or consists of a substitution of cytosine with thymine at a position corresponding to position 409 of SEQ ID NO: 4, C409T, as shown in SEQ ID NO: 6.

54. The nucleic acid construct according to paragraph 51, wherein the at least one nucleotide alteration comprises or consists of a substitution of cytosine with thymine at a position corresponding to position 495 of SEQ ID NO: 4, C409T, and of a substitution of thymine with cytosine at a position corresponding to position 411 of SEQ ID NO: 4.

55. The nucleic acid construct according to paragraph 51, wherein one, two or three of the nucleotides corresponding to position 409, 410 and/or 411 of SEQ ID NO: 4 are, independently from another, deleted.

56. The nucleic acid construct according to any one of paragraphs 33-55, wherein the at least one nucleotide alteration leads to a missense mutation and/or a premature stop codon.

57. The nucleic acid construct according to paragraph 56, wherein the premature stop codon is caused by a substitution of cytosine by thymine at a position corresponding to position 43 of SEQ ID NO: 3, C43T, as shown in SEQ ID NO: 13.

58. An expression vector comprising a nucleic acid construct according to any one of the paragraphs 33-57.

59. A fungal host cell comprising an expression vector or nucleic acid construct according to any one of the paragraphs 33-58.

60. The fungal host cell according to paragraph 59, wherein the host cell has an altered, reduced or eliminated Man₅GlcNAc₂-PP-Dol alpha-1,3-mannosyltransferase activity.

61. An isolated or purified polypeptide having altered, reduced or eliminated Man₅GlcNAc₂-PP-Dol alpha-1,3-mannosyltransferase activity, selected from the group consisting of:
(a) a polypeptide having at least 50% sequence identity to SEQ ID NO: 7 and comprising an alteration at a position corresponding to position 15, 17 or 137 of SEQ ID NO: 7;
(b) a polypeptide having at least 50% sequence identity to SEQ ID NO: 7 and comprising an alteration at a position corresponding to position 15, 17, 137 of SEQ ID NO: 7, wherein the alteration is one or more of R15*, T17I and L137F;
(c) a polypeptide that comprises, consists essentially of, or consists of SEQ ID NO: 8, SEQ ID NO: 15, or SEQ ID NO: 18;
(d) a polypeptide encoded by a polynucleotide that hybridizes under medium stringency conditions with the full-length complement of the mature polypeptide coding sequence of SEQ ID NO: 5, or the cDNA sequence thereof (SEQ ID NO: 6);
(e) a polypeptide encoded by a polynucleotide that hybridizes under medium stringency conditions with the full-length complement of the mature polypeptide coding sequence of SEQ ID NO: 13, or the cDNA sequence thereof (SEQ ID NO: 14);
(f) a polypeptide encoded by a polynucleotide that hybridizes under medium stringency conditions with the full-length complement of the mature polypeptide coding sequence of SEQ ID NO: 16, or the cDNA sequence thereof (SEQ ID NO: 17);
(g) a polypeptide encoded by a polynucleotide having at least 50% sequence identity to the mature polypeptide coding sequence of SEQ ID NO: 5, or the cDNA sequence thereof (SEQ ID NO: 6);
(h) a polypeptide encoded by a polynucleotide having at least 50% sequence identity to the mature polypeptide coding sequence of SEQ ID NO: 13, or the cDNA sequence thereof (SEQ ID NO: 14);
(i) a polypeptide encoded by a polynucleotide having at least 50% sequence identity to the mature polypeptide coding sequence of SEQ ID NO: 16, or the cDNA sequence thereof (SEQ ID NO: 17); and
(j) a polypeptide derived from a mature polypeptide of SEQ ID NO: 7 by substitution, deletion or addition of one or several amino acids in the mature polypeptide of SEQ ID NO: 7.

62. A whole broth formulation or cell culture composition comprising the polypeptide of paragraph 61, or the cell of any one of paragraphs 1-30 and 59-60.

63. A method of producing a polypeptide having glucoamylase activity, comprising cultivating the recombinant host cell of any one of paragraphs 1-30 and 59-60 under conditions conducive for production of the polypeptide.

64. The method of paragraph 63, further comprising recovering the polypeptide.

1-13. (canceled)

14: A fungal host cell comprising in its genome:
a) a first polynucleotide encoding a polypeptide of interest; and
b) a second polynucleotide encoding a polypeptide having a sequence identity of at least 60% but less than 100% to SEQ ID NO: 7 and comprising an alteration at a position corresponding to position 17 and/or 137 of SEQ ID NO: 7, or encoding a polypeptide having a sequence identity of at least 60% but less than 100% to SEQ ID NO: 21 and comprising an alteration at a position corresponding to position 19 and/or 139 of SEQ ID NO: 21.

15: The fungal host cell according to claim 14, wherein the alteration of said polypeptide at the position corresponding to position 17 or 137 of SEQ ID NO: 7, or corresponding to position 19 or 139 of SEQ ID NO: 21 is independently chosen from one another, an amino acid substitution, an amino acid insertion, an amino acid deletion, or a premature polypeptide termination.

16: The fungal host cell according to claim 14, wherein the alteration of said polypeptide at the position corresponding to position 17 or 137 of SEQ ID NO: 7 or corresponding to position 19 or 139 of SEQ ID NO: 21 is an amino acid substitution.

17: The fungal host cell according to claim 16, wherein the substitution is a substitution of leucine by phenylalanine at a position corresponding to position 137 of SEQ ID NO: 7 (L137F), or a substitution of leucine by phenylalanine at a position corresponding to position 139 of SEQ ID NO: 21 (L139F), or an amino acid substitution of threonine by isoleucine at a position corresponding to position 17 of SEQ ID NO: 7 (T17I), or an amino acid substitution of serine by isoleucine at a position corresponding to position 19 of SEQ ID NO: 21 (S19I).

18: The fungal host cell according to claim 14, wherein said fungal host cell is a yeast host cell.

19: The fungal host cell according to claim 14, wherein said fungal host cell is a filamentous fungal host cell.

20: The fungal host cell according to claim 19, wherein said filamentous fungal host cell is selected from an Aspergillus cell and a Trichoderma cell.

21: The fungal host cell according to claim 14, wherein the polypeptide of interest comprises an enzyme.

22: The fungal host cell according to claim 14, wherein the polypeptide of interest is a glycoprotein.

23: The fungal host cell according to claim 22, wherein the polypeptide of interest is a 1,4-alpha-glucosidase.

24: The fungal host cell according to claim 14, wherein the polypeptide of interest is a hydrolase.

25: The fungal host cell according to claim 24, wherein the polypeptide of interest is a lysozyme.

26: A method for producing a polypeptide of interest, the method comprising:
i) providing a fungal host cell according claim 14, and
ii) cultivating said host cell under conditions conducive for expression of the polypeptide of interest.

27: The method of claim 26, further comprising:
iii) recovering the polypeptide of interest.

28: A nucleic acid construct comprising a heterologous promoter operably linked to a polynucleotide encoding a polypeptide having a sequence identity of at least 60% but less than 100% to SEQ ID NO: 7, and comprising an alteration at a position corresponding to position 17 and/or 137 of SEQ ID NO: 7, or
encoding a polypeptide having a sequence identity of at least 60% but less than 100% to SEQ ID NO: 21, and comprising an alteration at a position corresponding to position 19 and/or 139 of SEQ ID NO: 21.

29: The nucleic acid construct according to claim 28, wherein the alteration at the position corresponding to position 17 and/or 137 of SEQ ID NO: 7 is a substitution.

30: The nucleic acid construct according to claim 28, wherein the alteration at the position corresponding to position 17 and/or 137 of SEQ ID NO: 7 is a substitution of leucine for phenylalanine at a position corresponding to position 137 of SEQ ID NO: 7 (L137F), and/or a substitution of threonine for isoleucine at a position corresponding to position 17 of SEQ ID NO: 7 (T17I).

31: The nucleic acid construct according to claim 28, wherein the alteration at the position corresponding to position 19 and/or 139 of SEQ ID NO: 21 is a substitution.

32: The nucleic acid construct according to claim 28, wherein the alteration at the position corresponding to position 19 and/or 139 of SEQ ID NO: 21 is a substitution of leucine for phenylalanine at a position corresponding to position 139 of SEQ ID NO: 21 (L139F), and/or a substitution of serine for isoleucine at a position corresponding to position 19 of SEQ ID NO: 21 (S19I).

33: An expression vector comprising a nucleic acid construct according to claim 28.


SEQUENCE LISTING (40 sequences)

SEQ ID NO: 1 - DNA, 20 bp, Artificial Sequence
  pIhar531 protospacer
gccgtacttgcttccgctgc

SEQ ID NO: 2 - DNA, 60 bp, Artificial Sequence
  pIhar531 primer IH160M-GT58
ttcgattcacggatgatgcagccgtacttgcttccgctgcgttttagagctagaaatagc

SEQ ID NO: 3 - DNA, 1400 bp, Aspergillus niger
atggactggatgcgcctaattcgcgatttgtgtttcaatccccgacacacaaaatggatggctccgctcctggtcctgggtgacgctttcctctgcgcgctgatcatctggaaagtgccctgtaaggctacagctaagctccgttcacacccttttgcgacaagtgaagcaatgccactaacctagccccgttgctattgtccacagataccgagattgactgggccacgtacatgcaacaaatatcgctttatttgtcaggagaacgcgattatactctcatcagaggatcaaccggtccccttgtctacccggccgcccatgtatacagttatacggccctctaccatctcaccgatgaggggcgcgatattttcttcggtcagatactatttgctgtgctctacttgatcacgctggtggttgtgctgtgctgttatagacagtcgggtgctccgccgtacttgcttccgctgctggtcctttccaagagacttcacagcgtttatgtcctgcgtctgttcaatgatggcttggcggcgctggcgatgtgggttgccattctgttattcatgaatcggaagtggacggctgcggtcgcagtgtggtctactggtgttgcgattaagatgacactgttgctgctggccccggctattgctgtggtcacggtgcttagtctgtcgcttggtcctagcgtggggctgggggttctggcggtgcttgtccaggtaggttcccatgaggctgtagggttggccaaaggcaatttgtgtgaagacttgtctgacattgaactacaggttttactcgcgataccgttcctacaaaacaacccggcggggtatctctcgcgggcgttcgagctaaccagacagttcatgtttaaatggacagtcaattggagatttgttggcgaagaagtattcttatctaagagcttttccctggcattgctggccgtccacattgtgctgctaggcgcttttgccgtcactggttggctgagatactccaggtctagcttgcctgcgttcattcggaatctgctagcgggtcgacatcgcacagtgtccctccccaaaccctacatcatgagcgtgatgctctcgtctctgacagttggcttgttgtgcgcaaggtcccttcattaccaattcttcgcctacctctcctgggcgacacccttcctcctctggcgcgcagggtttcatccaatcttgctgtaccttatctgggctatgcaagagtgggcttggaacacattccccagcaccaacctcagttccatcattgttgtcctctcacttgctacccagagtttcggcgtccttgcgaatagtgccagcgccttttataccatgcgttcgaaccctagcggtaaagagcataaccaatag

SEQ ID NO: 4 - DNA, 1242 bp, Artificial Sequence
  Alg3 wildtype cDNA
atggactggatgcgcctaattcgcgatttgtgtttcaatccccgacacacaaaatggatggctccgctcctggtcctgggtgacgctttcctctgcgcgctgatcatctggaaagtgccctataccgagattgactgggccacgtacatgcaacaaatatcgctttatttgtcaggagaacgcgattatactctcatcagaggatcaaccggtccccttgtctacccggccgcccatgtatacagttatacggccctctaccatctcaccgatgaggggcgcgatattttcttcggtcagatactatttgctgtgctctacttgatcacgctggtggttgtgctgtgctgttatagacagtcgggtgctccgccgtacttgcttccgctgctggtcctttccaagagacttcacagcgtttatgtcctgcgtctgttcaatgatggcttggcggcgctggcgatgtgggttgccattctgttattcatgaatcggaagtggacggctgcggtcgcagtgtggtctactggtgttgcgattaagatgacactgttgctgctggccccggctattgctgtggtcacggtgcttagtctgtcgcttggtcctagcgtggggctgggggttctggcggtgcttgtccaggttttactcgcgataccgttcctacaaaacaacccggcggggtatctctcgcgggcgttcgagctaaccagacagttcatgtttaaatggacagtcaattggagatttgttggcgaagaagtattcttatctaagagcttttccctggcattgctggccgtccacattgtgctgctaggcgcttttgccgtcactggttggctgagatactccaggtctagcttgcctgcgttcattcggaatctgctagcgggtcgacatcgcacagtgtccctccccaaaccctacatcatgagcgtgatgctctcgtctctgacagttggcttgttgtgcgcaaggtcccttcattaccaattcttcgcctacctctcctgggcgacacccttcctcctctggcgcgcagggtttcatccaatcttgctgtaccttatctgggctatgcaagagtgggcttggaacacattccccagcaccaacctcagttccatcattgttgtcctctcacttgctacccagagtttcggcgtccttgcgaatagtgccagcgccttttataccatgcgttcgaaccctagcggtaaagagcataaccaatag

SEQ ID NO: 5 - DNA, 1400 bp, Artificial Sequence
  Alg3 variant L137F gDNA
atggactggatgcgcctaattcgcgatttgtgtttcaatccccgacacacaaaatggatggctccgctcctggtcctgggtgacgctttcctctgcgcgctgatcatctggaaagtgccctgtaaggctacagctaagctccgttcacacccttttgcgacaagtgaagcaatgccactaacctagccccgttgctattgtccacagataccgagattgactgggccacgtacatgcaacaaatatcgctttatttgtcaggagaacgcgattatactctcatcagaggatcaaccggtccccttgtctacccggccgcccatgtatacagttatacggccctctaccatctcaccgatgaggggcgcgatattttcttcggtcagatactatttgctgtgctctacttgatcacgctggtggttgtgctgtgctgttatagacagtcgggtgctccgccgtacttgcttccgctgctcgtcctttccaagagatttcacagcgtttatgtcctgcgtctgttcaatgatggcttggcggcgctggcgatgtgggttgccattctgttattcatgaatcggaagtggacggctgcggtcgcagtgtggtctactggtgttgcgattaagatgacactgttgctgctggccccggctattgctgtggtcacggtgcttagtctgtcgcttggtcctagcgtggggctgggggttctggcggtgcttgtccaggtaggttcccatgaggctgtagggttggccaaaggcaatttgtgtgaagacttgtctgacattgaactacaggttttactcgcgataccgttcctacaaaacaacccggcggggtatctctcgcgggcgttcgagctaaccagacagttcatgtttaaatggacagtcaattggagatttgttggcgaagaagtattcttatctaagagcttttccctggcattgctggccgtccacattgtgctgctaggcgcttttgccgtcactggttggctgagatactccaggtctagcttgcctgcgttcattcggaatctgctagcgggtcgacatcgcacagtgtccctccccaaaccctacatcatgagcgtgatgctctcgtctctgacagttggcttgttgtgcgcaaggtcccttcattaccaattcttcgcctacctctcctgggcgacacccttcctcctctggcgcgcagggtttcatccaatcttgctgtaccttatctgggctatgcaagagtgggcttggaacacattccccagcaccaacctcagttccatcattgttgtcctctcacttgctacccagagtttcggcgtccttgcgaatagtgccagcgccttttataccatgcgttcgaaccctagcggtaaagagcataaccaatag

SEQ ID NO: 6 - DNA, 1242 bp, Artificial Sequence
  Alg3 variant L137F cDNA
atggactggatgcgcctaattcgcgatttgtgtttcaatccccgacacacaaaatggatggctccgctcctggtcctgggtgacgctttcctctgcgcgctgatcatctggaaagtgccctataccgagattgactgggccacgtacatgcaacaaatatcgctttatttgtcaggagaacgcgattatactctcatcagaggatcaaccggtccccttgtctacccggccgcccatgtatacagttatacggccctctaccatctcaccgatgaggggcgcgatattttcttcggtcagatactatttgctgtgctctacttgatcacgctggtggttgtgctgtgctgttatagacagtcgggtgctccgccgtacttgcttccgctgctcgtcctttccaagagatttcacagcgtttatgtcctgcgtctgttcaatgatggcttggcggcgctggcgatgtgggttgccattctgttattcatgaatcggaagtggacggctgcggtcgcagtgtggtctactggtgttgcgattaagatgacactgttgctgctggccccggctattgctgtggtcacggtgcttagtctgtcgcttggtcctagcgtggggctgggggttctggcggtgcttgtccaggttttactcgcgataccgttcctacaaaacaacccggcggggtatctctcgcgggcgttcgagctaaccagacagttcatgtttaaatggacagtcaattggagatttgttggcgaagaagtattcttatctaagagcttttccctggcattgctggccgtccacattgtgctgctaggcgcttttgccgtcactggttggctgagatactccaggtctagcttgcctgcgttcattcggaatctgctagcgggtcgacatcgcacagtgtccctccccaaaccctacatcatgagcgtgatgctctcgtctctgacagttggcttgttgtgcgcaaggtcccttcattaccaattcttcgcctacctctcctgggcgacacccttcctcctctggcgcgcagggtttcatccaatcttgctgtaccttatctgggctatgcaagagtgggcttggaacacattccccagcaccaacctcagttccatcattgttgtcctctcacttgctacccagagtttcggcgtccttgcgaatagtgccagcgccttttataccatgcgttcgaaccctagcggtaaagagcataaccaatag

SEQ ID NO: 7 - protein, 413 aa, Aspergillus niger
MDWMRLIRDLCFNPRHTKWMAPLLVLGDAFLCALIIWKVPYTEIDWATYMQQISLYLSGERDYTLIRGSTGPLVYPAAHVYSYTALYHLTDEGRDIFFGQILFAVLYLITLVVVLCCYRQSGAPPYLLPLLVLSKRLHSVYVLRLFNDGLAALAMWVAILLFMNRKWTAAVAVWSTGVAIKMTLLLLAPAIAVVTVLSLSLGPSVGLGVLAVLVQVLLAIPFLQNNPAGYLSRAFELTRQFMFKWTVNWRFVGEEVFLSKSFSLALLAVHIVLLGAFAVTGWLRYSRSSLPAFIRNLLAGRHRTVSLPKPYIMSVMLSSLTVGLLCARSLHYQFFAYLSWATPFLLWRAGFHPILLYLIWAMQEWAWNTFPSTNLSSIIVVLSLATQSFGVLANSASAFYTMRSNPSGKEHNQ

SEQ ID NO: 8 - protein, 413 aa, Artificial Sequence
  Alg3 variant L137F polypeptide
MDWMRLIRDLCFNPRHTKWMAPLLVLGDAFLCALIIWKVPYTEIDWATYMQQISLYLSGERDYTLIRGSTGPLVYPAAHVYSYTALYHLTDEGRDIFFGQILFAVLYLITLVVVLCCYRQSGAPPYLLPLLVLSKRFHSVYVLRLFNDGLAALAMWVAILLFMNRKWTAAVAVWSTGVAIKMTLLLLAPAIAVVTVLSLSLGPSVGLGVLAVLVQVLLAIPFLQNNPAGYLSRAFELTRQFMFKWTVNWRFVGEEVFLSKSFSLALLAVHIVLLGAFAVTGWLRYSRSSLPAFIRNLLAGRHRTVSLPKPYIMSVMLSSLTVGLLCARSLHYQFFAYLSWATPFLLWRAGFHPILLYLIWAMQEWAWNTFPSTNLSSIIVVLSLATQSFGVLANSASAFYTMRSNPSGKEHNQ

SEQ ID NO: 9 - protein, 573 aa, Artificial Sequence
  glucoamylase PE variant polypeptide
MYRFLVCALGLAASVLAQSVDSYVSSEGPIAKAGMLANIGPNGSKASGASAGVVVASPSKSDPDYWYTWTRDSSLVFKSLIDQYTTGIDSTSSLRTLIDDFVTAEANLQQVPNPSGTLTTGGLGEPKFNVDETAFTGPWGRPQRDGPALRSTALITYGNWLLSNGNTSYVTSNLWPIIQNDLGYVVSYWNQSTYDLWEEVDSSSFFTTAVQHRALREGAAFATAIGQTSQVSSYTTQADNLLCFLQSYWNPSGGYITANTGGGRSGKDANTLLASIHTYDPSAGCDAATFQPCSDKALSNLKVYVDSFRSVYSINSGVASNAAVATGRYPEDSYYGGNPWYLTTFAVAEQLYDALNVWESQGSLEVTSTSLAFFQQFSSGVTAGTYSSSSSTYSTLTSAIKNFADGFVAINAKYTPSNGGLAEQYSKSDGSPLSAVDLTWSYASALTAFEARNNTQFAGWGAAGLTVPSSCSGNSGGPTVAVTFNVNAETVWGENIYLTGSVDALENWSADNALLLSSANYPTWSITVNLPASTKIEYKYIRKMNGAVTWESDPNNSITTPASGSWTENDTWR

SEQ ID NO: 10 - DNA, 1722 bp, Artificial Sequence
  glucoamylase PE variant cDNA
atgtaccgcttccttgtctgtgcgctggggcttgcggcatcagttctcgcccagtcggtcgacagctatgttagcagcgaaggtcccatagccaaggcgggcatgcttgctaacattgggccgaacggctccaaggcctctggcgcatccgctggtgttgtggtcgcgagccctagcaagtcggaccccgactattggtacacttggacgcgtgactcgtccctcgtattcaagtcacttattgaccagtacaccaccggcatcgacagcacgagctctctgaggactctcatcgacgatttcgtaactgccgaggctaatctccagcaagtccccaaccctagtggtaccctcaccaccggtggcttgggagagcccaagttcaacgtcgacgaaactgcatttactggtccctggggtcgaccccaacgcgacggacctgccctccgctcgactgcattgatcacgtacggtaactggctgttgtccaacggaaatacgagctatgttacgagcaatctgtggccgatcatccagaacgaccttggttatgtcgtgtcatactggaaccagtctacctacgacctctgggaggaagtagactcgtcatcgttcttcactactgcagtacagcaccgtgctctccgtgaaggtgcggccttcgctaccgccatcggtcagacttcgcaggtcagcagctatacgactcaggcggacaatcttctgtgcttcttgcagtcttactggaacccgagcggtggttacatcactgctaacactggcggcggccgttccggcaaggatgccaacacacttctggcatccattcacacgtacgaccccagcgcgggctgcgacgctgcgactttccagccctgctctgacaaggcactgtcgaacctgaaggtctacgtcgactctttccgctcggtctactccatcaacagtggtgtcgcctctaacgctgccgtcgccacgggtcgttatcccgaggatagctactacggtggaaacccttggtacctcaccacatttgcggtcgccgagcaactctatgatgctctcaatgtctgggagtcgcagggttccctcgaggtcacctccacctcccttgccttcttccagcagttctcatccggcgtcactgctggcacctactcttctagctccagcacatacagcaccctcacgtctgccatcaagaactttgccgatggatttgtcgctatcaatgctaagtacacgccatccaacggtggcctggcggaacaatacagcaagagcgacggttctccccttagcgcggtggacttgacgtggagctacgcttcggctttgacggcgtttgaagcaaggaacaatactcagttcgccggctggggcgctgcaggcctgactgtgccttcctcttgctccggcaactctggtgggccgaccgttgctgtcacattcaacgtgaacgccgagactgtgtggggagagaacatctatcttactggttccgtcgatgctctggagaactggtcggccgacaatgccctcctgctctcatcggctaattacccgacctggagtatcaccgtcaacttgccggcgagcactaagattgagtacaagtacatccgcaaaatgaatggggccgttacctgggagtcagaccccaacaatagcatcactactccggccagcggctcgtggaccgagaatgacacttggcgttga

SEQ ID NO: 11 - protein, 573 aa, Gloeophyllum sepiarium
MYRFLVCALGLAASVLAQSVDSYVSSEGPIAKAGVLANIGPNGSKASGASAGVVVASPSTSDPDYWYTWTRDSSLVFKSLIDQYTTGIDSTSSLRTLIDDFVTAEANLQQVSNPSGTLTTGGLGEPKFNVDETAFTGAWGRPQRDGPALRSTALITYGNWLLSNGNTSYVTSNLWPIIQNDLGYVVSYWNQSTYDLWEEVDSSSFFTTAVQHRALREGAAFATAIGQTSQVSSYTTQADNLLCFLQSYWNPSGGYITANTGGGRSGKDANTLLASIHTYDPSAGCDAATFQPCSDKALSNLKVYVDSFRSVYSINSGVASNAAVATGRYPEDSYQGGNPWYLTTFAVAEQLYDALNVWESQGSLEVTSTSLAFFQQFSSGVTAGTYSSSSSTYSTLTSAIKNFADGFVAINAKYTPSNGGLAEQYSKSDGSPLSAVDLTWSYASALTAFEARNNTQFAGWGAAGLTVPSSCSGNSGGPTVAVTFNVNAETVWGENIYLTGSVDALENWSADNALLLSSANYPTWSITVNLPASTAIEYKYIRKNNGAVTWESDPNNSITTPASGSTTENDTWR

SEQ ID NO: 12 - DNA, 1722 bp, Gloeophyllum sepiarium
atgtaccgcttccttgtctgtgcgctggggcttgcggcatcagttctcgcccagtcggtcgacagctatgttagcagcgaaggtcccatagccaaggcgggcgtccttgctaacattgggccgaacggctccaaggcctctggcgcatccgctggtgttgtggtcgcgagccctagcacgtcggaccccgactattggtacacttggacgcgtgactcgtccctcgtattcaagtcacttattgaccagtacaccaccggcatcgacagcacgagctctctgaggactctcatcgacgatttcgtaactgccgaggctaatctccagcaagtctctaaccctagtggtaccctcaccaccggtggcttgggagagcccaagttcaacgtcgacgaaactgcatttactggtgcatggggtcgaccccaacgcgacggacctgccctccgctcgactgcattgatcacgtacggtaactggctgttgtccaacggaaatacgagctatgttacgagcaatctgtggccgatcatccagaacgaccttggttatgtcgtgtcatactggaaccagtctacctacgacctctgggaggaagtagactcgtcatcgttcttcactactgcagtacagcaccgtgctctccgtgaaggtgcggccttcgctaccgccatcggtcagacttcgcaggtcagcagctatacgactcaggcggacaatcttctgtgcttcttgcagtcttactggaacccgagcggtggttacatcactgctaacactggcggcggccgttccggcaaggatgccaacacacttctggcatccattcacacgtacgaccccagcgcgggctgcgacgctgcgactttccagccctgctctgacaaggcactgtcgaacctgaaggtctacgtcgactctttccgctcggtctactccatcaacagtggtgtcgcctctaacgctgccgtcgccacgggtcgttatcccgaggatagctaccagggtggaaacccttggtacctcaccacatttgcggtcgccgagcaactctatgatgctctcaatgtctgggagtcgcagggttccctcgaggtcacctccacctcccttgccttcttccagcagttctcatccggcgtcactgctggcacctactcttctagctccagcacatacagcaccctcacgtctgccatcaagaactttgccgatggatttgtcgctatcaatgctaagtacacgccatccaacggtggcctggcggaacaatacagcaagagcgacggttctccccttagcgcggtggacttgacgtggagctacgcttcggctttgacggcgtttgaagcaaggaacaatactcagttcgccggctggggcgctgcaggcctgactgtgccttcctcttgctccggcaactctggtgggccgaccgttgctgtcacattcaacgtgaacgccgagactgtgtggggagagaacatctatcttactggttccgtcgatgctctggagaactggtcggccgacaatgccctcctgctctcatcggctaattacccgacctggagtatcaccgtcaacttgccggcgagcactgctattgagtacaagtacatccgcaaaaataatggggccgttacctgggagtcagaccccaacaatagcatcactactccggccagcggctcgacgaccgagaatgacacttggcgttga

SEQ ID NO: 13 - DNA, 1400 bp, Artificial Sequence
  Alg3 variant R15* gDNA
atggactggatgcgcctaattcgcgatttgtgtttcaatccctgacacacaaaatggatggctccgctcctggtcctgggtgacgctttcctctgcgcgctgatcatctggaaagtgccctgtaaggctacagctaagctccgttcacacccttttgcgacaagtgaagcaatgccactaacctagccccgttgctattgtccacagataccgagattgactgggccacgtacatgcaacaaatatcgctttatttgtcaggagaacgcgattatactctcatcagaggatcaaccggtccccttgtctacccggccgcccatgtatacagttatacggccctctaccatctcaccgatgaggggcgcgatattttcttcggtcagatactatttgctgtgctctacttgatcacgctggtggttgtgctgtgctgttatagacagtcgggtgctccgccgtacttgcttccgctgctggtcctttccaagagacttcacagcgtttatgtcctgcgtctgttcaatgatggcttggcggcgctggcgatgtgggttgccattctgttattcatgaatcggaagtggacggctgcggtcgcagtgtggtctactggtgttgcgattaagatgacactgttgctgctggccccggctattgctgtggtcacggtgcttagtctgtcgcttggtcctagcgtggggctgggggttctggcggtgcttgtccaggtaggttcccatgaggctgtagggttggccaaaggcaatttgtgtgaagacttgtctgacattgaactacaggttttactcgcgataccgttcctacaaaacaacccggcggggtatctctcgcgggcgttcgagctaaccagacagttcatgtttaaatggacagtcaattggagatttgttggcgaagaagtattcttatctaagagcttttccctggcattgctggccgtccacattgtgctgctaggcgcttttgccgtcactggttggctgagatactccaggtctagcttgcctgcgttcattcggaatctgctagcgggtcgacatcgcacagtgtccctccccaaaccctacatcatgagcgtgatgctctcgtctctgacagttggcttgttgtgcgcaaggtcccttcattaccaattcttcgcctacctctcctgggcgacacccttcctcctctggcgcgcagggtttcatccaatcttgctgtaccttatctgggctatgcaagagtgggcttggaacacattccccagcaccaacctcagttccatcattgttgtcctctcacttgctacccagagtttcggcgtccttgcgaatagtgccagcgccttttataccatgcgttcgaaccctagcggtaaagagcataaccaatag

SEQ ID NO: 14 - DNA, 1242 bp, Artificial Sequence
  Alg3 variant R15* cDNA
atggactggatgcgcctaattcgcgatttgtgtttcaatccctgacacacaaaatggatggctccgctcctggtcctgggtgacgctttcctctgcgcgctgatcatctggaaagtgccctataccgagattgactgggccacgtacatgcaacaaatatcgctttatttgtcaggagaacgcgattatactctcatcagaggatcaaccggtccccttgtctacccggccgcccatgtatacagttatacggccctctaccatctcaccgatgaggggcgcgatattttcttcggtcagatactatttgctgtgctctacttgatcacgctggtggttgtgctgtgctgttatagacagtcgggtgctccgccgtacttgcttccgctgctggtcctttccaagagacttcacagcgtttatgtcctgcgtctgttcaatgatggcttggcggcgctggcgatgtgggttgccattctgttattcatgaatcggaagtggacggctgcggtcgcagtgtggtctactggtgttgcgattaagatgacactgttgctgctggccccggctattgctgtggtcacggtgcttagtctgtcgcttggtcctagcgtggggctgggggttctggcggtgcttgtccaggttttactcgcgataccgttcctacaaaacaacccggcggggtatctctcgcgggcgttcgagctaaccagacagttcatgtttaaatggacagtcaattggagatttgttggcgaagaagtattcttatctaagagcttttccctggcattgctggccgtccacattgtgctgctaggcgcttttgccgtcactggttggctgagatactccaggtctagcttgcctgcgttcattcggaatctgctagcgggtcgacatcgcacagtgtccctccccaaaccctacatcatgagcgtgatgctctcgtctctgacagttggcttgttgtgcgcaaggtcccttcattaccaattcttcgcctacctctcctgggcgacacccttcctcctctggcgcgcagggtttcatccaatcttgctgtaccttatctgggctatgcaagagtgggcttggaacacattccccagcaccaacctcagttccatcattgttgtcctctcacttgctacccagagtttcggcgtccttgcgaatagtgccagcgccttttataccatgcgttcgaaccctagcggtaaagagcataaccaatag

SEQ ID NO: 15 - protein, 14 aa, Artificial Sequence
  Alg3 variant R15* polypeptide
MDWMRLIRDLCFNP

SEQ ID NO: 16 - DNA, 1400 bp, Artificial Sequence
  Alg3 variant T17I gDNA
atggactggatgcgcctaattcgcgatttgtgtttcaatccccgacacataaaatggatggctccgctcctggtcctgggtgacgctttcctctgcgcgctgatcatctggaaagtgccctgtaaggctacagctaagctccgttcacacccttttgcgacaagtgaagcaatgccactaacctagccccgttgctattgtccacagataccgagattgactgggccacgtacatgcaacaaatatcgctttatttgtcaggagaacgcgattatactctcatcagaggatcaaccggtccccttgtctacccggccgcccatgtatacagttatacggccctctaccatctcaccgatgaggggcgcgatattttcttcggtcagatactatttgctgtgctctacttgatcacgctggtggttgtgctgtgctgttatagacagtcgggtgctccgccgtacttgcttccgctgctggtcctttccaagagacttcacagcgtttatgtcctgcgtctgttcaatgatggcttggcggcgctggcgatgtgggttgccattctgttattcatgaatcggaagtggacggctgcggtcgcagtgtggtctactggtgttgcgattaagatgacactgttgctgctggccccggctattgctgtggtcacggtgcttagtctgtcgcttggtcctagcgtggggctgggggttctggcggtgcttgtccaggtaggttcccatgaggctgtagggttggccaaaggcaatttgtgtgaagacttgtctgacattgaactacaggttttactcgcgataccgttcctacaaaacaacccggcggggtatctctcgcgggcgttcgagctaaccagacagttcatgtttaaatggacagtcaattggagatttgttggcgaagaagtattcttatctaagagcttttccctggcattgctggccgtccacattgtgctgctaggcgcttttgccgtcactggttggctgagatactccaggtctagcttgcctgcgttcattcggaatctgctagcgggtcgacatcgcacagtgtccctccccaaaccctacatcatgagcgtgatgctctcgtctctgacagttggcttgttgtgcgcaaggtcccttcattaccaattcttcgcctacctctcctgggcgacacccttcctcctctggcgcgcagggtttcatccaatcttgctgtaccttatctgggctatgcaagagtgggcttggaacacattccccagcaccaacctcagttccatcattgttgtcctctcacttgctacccagagtttcggcgtccttgcgaatagtgccagcgccttttataccatgcgttcgaaccctagcggtaaagagcataaccaatag

SEQ ID NO: 17 - DNA, 1242 bp, Artificial Sequence
  Alg3 variant T17I cDNA
atggactggatgcgcctaattcgcgatttgtgtttcaatccccgacacataaaatggatggctccgctcctggtcctgggtgacgctttcctctgcgcgctgatcatctggaaagtgccctataccgagattgactgggccacgtacatgcaacaaatatcgctttatttgtcaggagaacgcgattatactctcatcagaggatcaaccggtccccttgtctacccggccgcccatgtatacagttatacggccctctaccatctcaccgatgaggggcgcgatattttcttcggtcagatactatttgctgtgctctacttgatcacgctggtggttgtgctgtgctgttatagacagtcgggtgctccgccgtacttgcttccgctgctggtcctttccaagagacttcacagcgtttatgtcctgcgtctgttcaatgatggcttggcggcgctggcgatgtgggttgccattctgttattcatgaatcggaagtggacggctgcggtcgcagtgtggtctactggtgttgcgattaagatgacactgttgctgctggccccggctattgctgtggtcacggtgcttagtctgtcgcttggtcctagcgtggggctgggggttctggcggtgcttgtccaggttttactcgcgataccgttcctacaaaacaacccggcggggtatctctcgcgggcgttcgagctaaccagacagttcatgtttaaatggacagtcaattggagatttgttggcgaagaagtattcttatctaagagcttttccctggcattgctggccgtccacattgtgctgctaggcgcttttgccgtcactggttggctgagatactccaggtctagcttgcctgcgttcattcggaatctgctagcgggtcgacatcgcacagtgtccctccccaaaccctacatcatgagcgtgatgctctcgtctctgacagttggcttgttgtgcgcaaggtcccttcattaccaattcttcgcctacctctcctgggcgacacccttcctcctctggcgcgcagggtttcatccaatcttgctgtaccttatctgggctatgcaagagtgggcttggaacacattccccagcaccaacctcagttccatcattgttgtcctctcacttgctacccagagtttcggcgtccttgcgaatagtgccagcgccttttataccatgcgttcgaaccctagcggtaaagagcataaccaatag

SEQ ID NO: 18 - protein, 413 aa, Artificial Sequence
  Alg3 variant T17I polypeptide
MDWMRLIRDLCFNPRHIKWMAPLLVLGDAFLCALIIWKVPYTEIDWATYMQQISLYLSGERDYTLIRGSTGPLVYPAAHVYSYTALYHLTDEGRDIFFGQILFAVLYLITLVVVLCCYRQSGAPPYLLPLLVLSKRLHSVYVLRLFNDGLAALAMWVAILLFMNRKWTAAVAVWSTGVAIKMTLLLLAPAIAVVTVLSLSLGPSVGLGVLAVLVQVLLAIPFLQNNPAGYLSRAFELTRQFMFKWTVNWRFVGEEVFLSKSFSLALLAVHIVLLGAFAVTGWLRYSRSSLPAFIRNLLAGRHRTVSLPKPYIMSVMLSSLTVGLLCARSLHYQFFAYLSWATPFLLWRAGFHPILLYLIWAMQEWAWNTFPSTNLSSIIVVLSLATQSFGVLANSASAFYTMRSNPSGKEHNQ

SEQ ID NO: 19 - DNA, 21 bp, Artificial Sequence
  pTNA469 protospacer
aatccccgacacacaaaatgg

SEQ ID NO: 20 - DNA, 838 bp, Acremonium alcalophilum
atgaagcttcttccctccttgattggcctggccagtctggcgtccctcgccgtcgcccggatccccggctttgacatttcgggctggcaaccgaccaccgactttgcaagggcgtatgctaatggagatcgtttcgtctacatcaaggtacgttcaaccttgccaccaagttgcgaacccgagacaagactgtgaccgcctcctttgccctggggcagctcacgcacccagcagcatcccatcccccggccccccacgtaccaccggaaagctaacatcaaccccctaccactgctaccaggccaccgagggcaccacattcaagagctccgcattcagccgccagtacaccggcgcaacgcaaaacggcttcatccgcggcgcctaccacttcgcccagcccgccgcgtcctcgggcgccgcgcaggcgagatacttcgccagcaacggcggcggctggtccaaggacggcatcaccctgcccggggcgctggacatcgagtacaaccccaacggcgccacctgctacggcctctcgcaatcggccatggtgaactggatcgaggactttgtcaccacctaccacggcatcacctcccgctggcccgtcatctacaccaccaccgactggtggacccagtgcaccggcaactccaaccgcttcgcgaaccgctgcccgctgtggatcgcccgctacgccagctccgtcggcactctgcccaatggctggggcttttacaccttctggcagtacaacgacaagtatcctcagggcggtgattcgaactggttcaacggcgatgcgtcgcgtctcagggctctcgctaacggagactaa

SEQ ID NO: 21 - protein, 420 aa, Trichoderma reesei
MASLIKTAVDIANGRHALSRYVIFGLWLADAVLCGLIIWKVPYTEIDWVAYMEQVTQFVHGERDYPKMEGGTGPLVYPAAHVYIYTGLYYLTNKGTDILLAQQLFAVLYMATLAVVMTCYSKAKVPPYIFPLLILSKRLHSVFVLRCFNDCFAAFFLWLCIFFFQRREWTIGALAYSIGLGVKMSLLLVLPAVVIVLYLGRGFKGALRLLWLMVQVQLLLAIPFITTNWRGYLGRAFELSRQFKFEWTVNWRMLGEDLFLSRGFSITLLAFHAIFLLAFILGRWLKIRERTVLGMIPYVIRFRSPFTEQEERAISNRVVTPGYVMSTILSANVVGLLFARSLHYQFYAYLAWATPYLLWTACPNLLVVAPLWAAQEWAWNVFPSTPLSSSVVVSVLAVTVAMAFAGSNPQPRETSKPKQH

SEQ ID NO: 22 - DNA, 60 bp, Artificial Sequence
  1238937 oligo
tgcaggaatttctactcttgtagatggctctggcttgcggatgcggtttttttggctctt

SEQ ID NO: 23 - DNA, 60 bp, Artificial Sequence
  1238938 oligo
tgcaggaatttctactcttgtagatgagaggatgagaagcgggaagtttttttggctctt

SEQ ID NO: 24 - DNA, 21 bp, Artificial Sequence
  pAgJg341 protospacer
ggctctggcttgcggatgcgg

SEQ ID NO: 25 - DNA, 21 bp, Artificial Sequence
  pAgJg342 protospacer
gagaggatgagaagcgggaag

SEQ ID NO: 26 - DNA, 94 bp, Artificial Sequence
  1238939 oligo
atcaaaactgccgtggacattgccaacggccgccatgcgctgatcagatatgtcatcttcgggctctggcttgcggatgcggtgctgtgcgggc

SEQ ID NO: 27 - DNA, 94 bp, Artificial Sequence
  1238940 oligo
gcccgcacagcaccgcatccgcaagccagagcccgaagatgacatatctgatcagcgcatggcggccgttggcaatgtccacggcagttttgat

SEQ ID NO: 28 - DNA, 1504 bp, Trichoderma reesei
atggcgtcactcatcaaaactgccgtggacattgccaacggccgccatgcgctgtccagatatgtcatctttgggctctggcttgcggatgcggtgctgtgcgggctgattatctggaaagtgccttgtcagtctcacccatgaatgacccattcctttgcaaccccaagaattgtcactctgaacatcggagaaaagggggggaaacacatcaagtcgtcaaaagaagacatctctacgaatgaaagacacagaactcgtgtatgaagctgaggctgatcaatctctagatacggaaatcgactgggtcgcctacatggagcaagtcacccagttcgtccacggagagcgagactaccccaagatggagggcggcacagggcccctggtgtatcccgcggcccatgtgtacatctacacagggctctactacctgacgaacaagggcaccgacatcctgctggcgcagcagctctttgccgtgctctacatggctactctggcggtcgtcatgacatgctactccaaggccaaggtgagctgagccgagggtgttcacgtttaagagcaacaagtcactgaaaccggccatcaaacacccccccatggacaggtcccgccgtacatcttcccgcttctcatcctctccaaaagacttcacagcgtcttcgtcctgagatgcttcaacgactgcttcgccgccttcttcctctggctctgcatcttcttcttccagaggcgagagtggaccatcggagctctcgcatacagcatcggcctgggcgtcaaaatgtcgctgctactggttctccccgccgtggtcatcgtcctctacctcggccgcggcttcaagggcgccctgcggctgctctggctcatggtgcaggtccagctcctcctcgccatacccttcatcacgacaaattggcgcggctacctcggccgtgcattcgagctctcgaggcagttcaagtttgaatggacagtcaattggcgcatgctgggcgaggatctgttcctcagccggggcttctctatcacgctactggcatttcacgccatcttcctcctcgcctttatcctcggccggtggctgaagattagggaacggaccgtactcgggatgatcccctatgtcatccgattcagatcgccctttaccgagcaggaagagcgcgccatctccaaccgcgtcgtcacgcccggctatgtcatgtccaccatcttgtcggccaacgtggtgggactgctgtttgcccggtctctgcactaccagttctatgcatatctggcgtgggcgaccccctatctcctgtggacggcctgccccaatcttttggtggtggcccccctctgggcggcgcaagaatgggcctggaacgtcttccccagcacgcctcttagctcgagcgtcgtggtgagcgtgctggccgtgacggtggccatggcgtttgcaggttcaaatccgcagccacgtgaaacatcgaagccgaagcagcactaa

SEQ ID NO: 29 - DNA, 26 bp, Artificial Sequence
  1238985 primer
caagcctcatgttttgccaacggaac

SEQ ID NO: 30 - DNA, 22 bp, Artificial Sequence
  1238986 primer
ccctaatcttcagccaccggcc

SEQ ID NO: 31 - DNA, 94 bp, Artificial Sequence
  1238941 primer
gtcccgccgtacatcttcccgcttctcatcctctccaagagatttcacagcgtcttcgtcctgagatgcttcaacgactgcttcgccgccttct

SEQ ID NO: 32 - DNA, 94 bp, Artificial Sequence
  1238942 primer
agaaggcggcgaagcagtcgttgaagcatctcaggacgaagacgctgtgaaatctcttggagaggatgagaagcgggaagatgtacggcgggac

SEQ ID NO: 33 - protein, 227 aa, Acremonium alcalophilum
MKLLPSLIGLASLASLAVARIPGFDISGWQPTTDFARAYANGDRFVYIKATEGTTFKSSAFSRQYTGATQNGFIRGAYHFAQPAASSGAAQARYFASNGGGWSKDGITLPGALDIEYNPNGATCYGLSQSAMVNWIEDFVTTYHGITSRWPVIYTTTDWWTQCTGNSNRFANRCPLWIARYASSVGTLPNGWGFYTFWQYNDKYPQGGDSNWFNGDASRLRALANGD

SEQ ID NO: 34 - DNA, 1504 bp, Artificial Sequence
  S19I Alg3 T. reesei variant
atggcgtcactcatcaaaactgccgtggacattgccaacggccgccatgcgctgatcagatatgtcatcttcgggctctggcttgcggatgcggtgctgtgcgggctgattatctggaaagtgccttgtcagtctcacccatgaatgacccattcctttgcaaccccaagaattgtcactctgaacatcggagaaaagggggggaaacacatcaagtcgtcaaaagaagacatctctacgaatgaaagacacagaactcgtgtatgaagctgaggctgatcaatctctagatacggaaatcgactgggtcgcctacatggagcaagtcacccagttcgtccacggagagcgagactaccccaagatggagggcggcacagggcccctggtgtatcccgcggcccatgtgtacatctacacagggctctactacctgacgaacaagggcaccgacatcctgctggcgcagcagctctttgccgtgctctacatggctactctggcggtcgtcatgacatgctactccaaggccaaggtgagctgagccgagggtgttcacgtttaagagcaacaagtcactgaaaccggccatcaaacacccccccatggacaggtcccgccgtacatcttcccgcttctcatcctctccaaaagacttcacagcgtcttcgtcctgagatgcttcaacgactgcttcgccgccttcttcctctggctctgcatcttcttcttccagaggcgagagtggaccatcggagctctcgcatacagcatcggcctgggcgtcaaaatgtcgctgctactggttctccccgccgtggtcatcgtcctctacctcggccgcggcttcaagggcgccctgcggctgctctggctcatggtgcaggtccagctcctcctcgccatacccttcatcacgacaaattggcgcggctacctcggccgtgcattcgagctctcgaggcagttcaagtttgaatggacagtcaattggcgcatgctgggcgaggatctgttcctcagccggggcttctctatcacgctactggcatttcacgccatcttcctcctcgcctttatcctcggccggtggctgaagattagggaacggaccgtactcgggatgatcccctatgtcatccgattcagatcgccctttaccgagcaggaagagcgcgccatctccaaccgcgtcgtcacgcccggctatgtcatgtccaccatcttgtcggccaacgtggtgggactgctgtttgcccggtctctgcactaccagttctatgcatatctggcgtgggcgaccccctatctcctgtggacggcctgccccaatcttttggtggtggcccccctctgggcggcgcaagaatgggcctggaacgtcttccccagcacgcctcttagctcgagcgtcgtggtgagcgtgctggccgtgacggtggccatggcgtttgcaggttcaaatccgcagccacgtgaaacatcgaagccgaagcagcactaa

SEQ ID NO: 35 - DNA, 1263 bp, Artificial Sequence
  S19I Alg3 T. reesei variant
atggcgtcactcatcaaaactgccgtggacattgccaacggccgccatgcgctgatcagatatgtcatcttcgggctctggcttgcggatgcggtgctgtgcgggctgattatctggaaagtgccttatacggaaatcgactgggtcgcctacatggagcaagtcacccagttcgtccacggagagcgagactaccccaagatggagggcggcacagggcccctggtgtatcccgcggcccatgtgtacatctacacagggctctactacctgacgaacaagggcaccgacatcctgctggcgcagcagctctttgccgtgctctacatggctactctggcggtcgtcatgacatgctactccaaggccaaggtcccgccgtacatcttcccgcttctcatcctctccaaaagacttcacagcgtcttcgtcctgagatgcttcaacgactgcttcgccgccttcttcctctggctctgcatcttcttcttccagaggcgagagtggaccatcggagctctcgcatacagcatcggcctgggcgtcaaaatgtcgctgctactggttctccccgccgtggtcatcgtcctctacctcggccgcggcttcaagggcgccctgcggctgctctggctcatggtgcaggtccagctcctcctcgccatacccttcatcacgacaaattggcgcggctacctcggccgtgcattcgagctctcgaggcagttcaagtttgaatggacagtcaattggcgcatgctgggcgaggatctgttcctcagccggggcttctctatcacgctactggcatttcacgccatcttcctcctcgcctttatcctcggccggtggctgaagattagggaacggaccgtactcgggatgatcccctatgtcatccgattcagatcgccctttaccgagcaggaagagcgcgccatctccaaccgcgtcgtcacgcccggctatgtcatgtccaccatcttgtcggccaacgtggtgggactgctgtttgcccggtctctgcactaccagttctatgcatatctggcgtgggcgaccccctatctcctgtggacggcctgccccaatcttttggtggtggcccccctctgggcggcgcaagaatgggcctggaacgtcttccccagcacgcctcttagctcgagcgtcgtggtgagcgtgctggccgtgacggtggccatggcgtttgcaggttcaaatccgcagccacgtgaaacatcgaagccgaagcagcactaa

SEQ ID NO: 36 - protein, 420 aa, Artificial Sequence
  S19I Alg3 T. reesei variant
MASLIKTAVDIANGRHALIRYVIFGLWLADAVLCGLIIWKVPYTEIDWVAYMEQVTQFVHGERDYPKMEGGTGPLVYPAAHVYIYTGLYYLTNKGTDILLAQQLFAVLYMATLAVVMTCYSKAKVPPYIFPLLILSKRLHSVFVLRCFNDCFAAFFLWLCIFFFQRREWTIGALAYSIGLGVKMSLLLVLPAVVIVLYLGRGFKGALRLLWLMVQVQLLLAIPFITTNWRGYLGRAFELSRQFKFEWTVNWRMLGEDLFLSRGFSITLLAFHAIFLLAFILGRWLKIRERTVLGMIPYVIRFRSPFTEQEERAISNRVVTPGYVMSTILSANVVGLLFARSLHYQFYAYLAWATPYLLWTACPNLLVVAPLWAAQEWAWNVFPSTPLSSSVVVSVLAVTVAMAFAGSNPQPRETSKPKQH

SEQ ID NO: 37 - DNA, 1504 bp, Artificial Sequence
  L139F Alg3 T. reesei variant
atggcgtcactcatcaaaactgccgtggacattgccaacggccgccatgcgctgtccagatatgtcatctttgggctctggcttgcggatgcggtgctgtgcgggctgattatctggaaagtgccttgtcagtctcacccatgaatgacccattcctttgcaaccccaagaattgtcactctgaacatcggagaaaagggggggaaacacatcaagtcgtcaaaagaagacatctctacgaatgaaagacacagaactcgtgtatgaagctgaggctgatcaatctctagatacggaaatcgactgggtcgcctacatggagcaagtcacccagttcgtccacggagagcgagactaccccaagatggagggcggcacagggcccctggtgtatcccgcggcccatgtgtacatctacacagggctctactacctgacgaacaagggcaccgacatcctgctggcgcagcagctctttgccgtgctctacatggctactctggcggtcgtcatgacatgctactccaaggccaaggtgagctgagccgagggtgttcacgtttaagagcaacaagtcactgaaaccggccatcaaacacccccccatggacaggtcccgccgtacatcttcccgcttctcatcctctccaagagatttcacagcgtcttcgtcctgagatgcttcaacgactgcttcgccgccttcttcctctggctctgcatcttcttcttccagaggcgagagtggaccatcggagctctcgcatacagcatcggcctgggcgtcaaaatgtcgctgctactggttctccccgccgtggtcatcgtcctctacctcggccgcggcttcaagggcgccctgcggctgctctggctcatggtgcaggtccagctcctcctcgccatacccttcatcacgacaaattggcgcggctacctcggccgtgcattcgagctctcgaggcagttcaagtttgaatggacagtcaattggcgcatgctgggcgaggatctgttcctcagccggggcttctctatcacgctactggcatttcacgccatcttcctcctcgcctttatcctcggccggtggctgaagattagggaacggaccgtactcgggatgatcccctatgtcatccgattcagatcgccctttaccgagcaggaagagcgcgccatctccaaccgcgtcgtcacgcccggctatgtcatgtccaccatcttgtcggccaacgtggtgggactgctgtttgcccggtctctgcactaccagttctatgcatatctggcgtgggcgaccccctatctcctgtggacggcctgccccaatcttttggtggtggcccccctctgggcggcgcaagaatgggcctggaacgtcttccccagcacgcctcttagctcgagcgtcgtggtgagcgtgctggccgtgacggtggccatggcgtttgcaggttcaaatccgcagccacgtgaaacatcgaagccgaagcagcactaa

SEQ ID NO: 38 - DNA, 1263 bp, Artificial Sequence
  L139F Alg3 T. reesei variant
atggcgtcactcatcaaaactgccgtggacattgccaacggccgccatgcgctgtccagatatgtcatctttgggctctggcttgcggatgcggtgctgtgcgggctgattatctggaaagtgccttatacggaaatcgactgggtcgcctacatggagcaagtcacccagttcgtccacggagagcgagactaccccaagatggagggcggcacagggcccctggtgtatcccgcggcccatgtgtacatctacacagggctctactacctgacgaacaagggcaccgacatcctgctggcgcagcagctctttgccgtgctctacatggctactctggcggtcgtcatgacatgctactccaaggccaaggtcccgccgtacatcttcccgcttctcatcctctccaagagatttcacagcgtcttcgtcctgagatgcttcaacgactgcttcgccgccttcttcctctggctctgcatcttcttcttccagaggcgagagtggaccatcggagctctcgcatacagcatcggcctgggcgtcaaaatgtcgctgctactggttctccccgccgtggtcatcgtcctctacctcggccgcggcttcaagggcgccctgcggctgctctggctcatggtgcaggtccagctcctcctcgccatacccttcatcacgacaaattggcgcggctacctcggccgtgcattcgagctctcgaggcagttcaagtttgaatggacagtcaattggcgcatgctgggcgaggatctgttcctcagccggggcttctctatcacgctactggcatttcacgccatcttcctcctcgcctttatcctcggccggtggctgaagattagggaacggaccgtactcgggatgatcccctatgtcatccgattcagatcgccctttaccgagcaggaagagcgcgccatctccaaccgcgtcgtcacgcccggctatgtcatgtccaccatcttgtcggccaacgtggtgggactgctgtttgcccggtctctgcactaccagttctatgcatatctggcgtgggcgaccccctatctcctgtggacggcctgccccaatcttttggtggtggcccccctctgggcggcgcaagaatgggcctggaacgtcttccccagcacgcctcttagctcgagcgtcgtggtgagcgtgctggccgtgacggtggccatggcgtttgcaggttcaaatccgcagccacgtgaaacatcgaagccgaagcagcactaa

SEQ ID NO: 39 - protein, 420 aa, Artificial Sequence
  L139F Alg3 T. reesei variant
MASLIKTAVDIANGRHALSRYVIFGLWLADAVLCGLIIWKVPYTEIDWVAYMEQVTQFVHGERDYPKMEGGTGPLVYPAAHVYIYTGLYYLTNKGTDILLAQQLFAVLYMATLAVVMTCYSKAKVPPYIFPLLILSKRFHSVFVLRCFNDCFAAFFLWLCIFFFQRREWTIGALAYSIGLGVKMSLLLVLPAVVIVLYLGRGFKGALRLLWLMVQVQLLLAIPFITTNWRGYLGRAFELSRQFKFEWTVNWRMLGEDLFLSRGFSITLLAFHAIFLLAFILGRWLKIRERTVLGMIPYVIRFRSPFTEQEERAISNRVVTPGYVMSTILSANVVGLLFARSLHYQFYAYLAWATPYLLWTACPNLLVVAPLWAAQEWAWNVFPSTPLSSSVVVSVLAVTVAMAFAGSNPQPRETSKPKQH

SEQ ID NO: 40 - DNA, 1263 bp, Artificial Sequence
  WT Alg3 cDNA T. reesei
atggcgtcactcatcaaaactgccgtggacattgccaacggccgccatgcgctgtccagatatgtcatctttgggctctggcttgcggatgcggtgctgtgcgggctgattatctggaaagtgccttatacggaaatcgactgggtcgcctacatggagcaagtcacccagttcgtccacggagagcgagactaccccaagatggagggcggcacagggcccctggtgtatcccgcggcccatgtgtacatctacacagggctctactacctgacgaacaagggcaccgacatcctgctggcgcagcagctctttgccgtgctctacatggctactctggcggtcgtcatgacatgctactccaaggccaaggtcccgccgtacatcttcccgcttctcatcctctccaaaagacttcacagcgtcttcgtcctgagatgcttcaacgactgcttcgccgccttcttcctctggctctgcatcttcttcttccagaggcgagagtggaccatcggagctctcgcatacagcatcggcctgggcgtcaaaatgtcgctgctactggttctccccgccgtggtcatcgtcctctacctcggccgcggcttcaagggcgccctgcggctgctctggctcatggtgcaggtccagctcctcctcgccatacccttcatcacgacaaattggcgcggctacctcggccgtgcattcgagctctcgaggcagttcaagtttgaatggacagtcaattggcgcatgctgggcgaggatctgttcctcagccggggcttctctatcacgctactggcatttcacgccatcttcctcctcgcctttatcctcggccggtggctgaagattagggaacggaccgtactcgggatgatcccctatgtcatccgattcagatcgccctttaccgagcaggaagagcgcgccatctccaaccgcgtcgtcacgcccggctatgtcatgtccaccatcttgtcggccaacgtggtgggactgctgtttgcccggtctctgcactaccagttctatgcatatctggcgtgggcgaccccctatctcctgtggacggcctgccccaatcttttggtggtggcccccctctgggcggcgcaagaatgggcctggaacgtcttccccagcacgcctcttagctcgagcgtcgtggtgagcgtgctggccgtgacggtggccatggcgtttgcaggttcaaatccgcagccacgtgaaacatcgaagccgaagcagcactaa